(19) United States
(12) Patent Application Publication
(10) Pub. No.: US 2020/0030242 A1
(43) Pub. Date: Jan. 30, 2020

(54) ORAL DOSAGE FORM WITH DRYING AGENT FOR DELIVERY OF ACTIVE AGENT

(71) Applicant: Entrega Inc., Boston, MA (US)

(72) Inventors: Daniel BONNER, Briantree, MA (US); John JANTZ, Arlington, MA (US); Peter TRAN, Tampa, FL (US); Rachel SHAPIRO, Yorktown, NY (US); Nicholas PILLA, Boston, MA (US); Jules JACOB, Yardley, PA (US); Braden KUO, Newton, MA (US); Colin Rober GARDNER, Concord, MA (US); Padmaja SHIVANAND, Seattle, WA (US); Jacob BARLOW, Boston, MA (US); Lisa Viana FERREIRA, Franklin, MA (US)

(21) Appl. No.: 16/071,961

(22) PCT Filed: Feb. 3, 2017
(86) PCT No.: PCT/US2017/016539

§ 371 Date: Jul. 23, 2018

Related U.S. Application Data
(60) Provisional application No. 62/291,812, filed on Feb. 5, 2016.
(60) Provisional application No. 62/382,362, filed on Sep. 1, 2016.

(51) Int. Cl. A61K 9/20; A61K 9/28; A61K 9/50

(52) U.S. Cl. CPC A61K 9/2086; A61K 38/23; A61K 9/5084; A61K 9/2846

(57) ABSTRACT

A pharmaceutically acceptable oral dosage form for delivery of an agent to an intestinal site has one or more active agent regions having an active agent to be delivered to the intestinal site, one or more drying agent regions having at least one drying agent therein capable of drying an area about the intestinal site, the one or more drying agent regions being separate from the one or more active agent regions, and a protective coating at least partially covering a surface of the form. The dosage form has a fluid uptake capacity as measured for the entire dosage form when immersed in a fluid media according to a Dosage Form Fluid Uptake Assay of at least about 20 g fluid per dosage form.

Oral dosing of active agents is attractive for many reasons, including ease of administration and high patient compliance. However, for some active agents, such as poorly absorbed, sensitive (i.e., pH sensitive, enzyme-sensitive, and the like), and/or high molecular weight active agents, oral dosing may be less effective or ineffective for achieving sufficient blood concentration of the active agent as compared to alternative dosing strategies. For example, active agents such as proteins and other macromolecules may be enzymatically degraded in the gastrointestinal tract and/or may have limited transport across the intestinal epithelium.

One potential strategy for circumventing the hostile environment of the gastrointestinal tract is to alter the environment through the use of protease inhibitors and/or derivatization of agents with polyethylene glycol to prevent enzymatic degradation. Another potential strategy is to increase the permeability of the tissue in the gastrointestinal tract such that absorption of an agent increases. An agent may be formulated with an excipient that can, for example, open the tight junctions of the intestine to allow an agent to pass through the intestinal epithelium. A further approach to improving delivery of an agent in the gastrointestinal tract is to apply an enteric coating to the agent such that the agent is not exposed to the harsh pH conditions of the stomach, as is instead released in the small intestine, where absorption occurs more readily.

While significant progress has been made in the development of such forms for the delivery of active agents to the gastrointestinal tract, a need remains for improved forms that can be orally administered and that provides active agents to the gastrointestinal tract in a form that allows the active agent to be readily absorbed by the intestinal tissue, without excessive degradation thereof.

One aspect of the present disclosure is a pharmaceutically acceptable oral dosage form for delivery of an agent to an intestinal site. The oral dosage form includes an active agent to be delivered to the intestinal site, at least one drying agent capable of drying an area about the intestinal site, and a protective coating covering a surface of the form. The dosage form has a total drying agent content of at least about 15% by weight. The oral dosage form may provide a drying effect in an area about the intestinal site where the active agent is to be delivered, thereby enhancing the bioavailability of the active agent. Methods of administering the active agent with such an oral dosage form, as well as methods of manufacturing the oral dosage form, are also provided.

Another aspect of the present disclosure is a pharmaceutically acceptable oral dosage form for delivery of an agent to an intestinal site has one or more active agent regions having an active agent to be delivered to the intestinal site, one or more drying agent regions having at least one drying agent therein capable of drying an area about the intestinal site, the one or more drying agent regions being separate from the one or more active agent regions, and a protective coating at least partially covering a surface of the form. The dosage form has a fluid uptake capacity as measured for the entire dosage form when immersed in a fluid media according to a Dosage Form Fluid Uptake Assay of at least about 20 g fluid per dosage form.

Other aspects, features and embodiments of the present disclosure will be, in part, discussed and, in part, apparent in the following description.

### BRIEF DESCRIPTION OF THE FIGURES

FIG. 1A shows one embodiment of an oral dosage form comprising a bilayer tablet having a drying agent according to aspects of the present disclosure;

FIG. 1B shows an embodiment of an oral dosage form comprising a trilayer tablet having a drying agent according to aspects of the present disclosure;

FIG. 1C shows an embodiment of an oral dosage form comprising a bilayer tablet having a drying agent and a barrier layer according to aspects of the present disclosure;

FIG. 1D shows an embodiment of an oral dosage form comprising a tablet with concentric layers and having a drying agent according to aspects of the present disclosure;

FIG. 2 shows an embodiment of an oral dosage form comprising a capsule having a drying agent according to aspects of the present disclosure;

FIGS. 3A-3E show further embodiments of an oral dosage form comprising a capsule having a drying agent according to aspects of the present disclosure;

FIG. 4 shows results for a Surgical Bioavailability Assay performed for an oral dosage form according to aspects of the present disclosure;

FIG. 5 shows results for an Endoscopic Bioavailability Assay performed for an oral dosage form according to aspects of the present disclosure;

FIG. 6 shows another embodiment of an oral dosage form comprising a capsule having a drying agent according to aspects of the present disclosure;

FIGS. 7A-7F show further embodiments of oral dosage forms comprising a drying agent according to aspects of the present disclosure;

FIG. 8 shows results for a Port Bioavailability Assay performed for oral dosage forms that demonstrates the synergy of combining drying agent with permeation enhancer in oral dosage forms.

Other aspects, embodiments and features of the inventive subject matter will become apparent from the following detailed description when considered in conjunction with the accompanying drawing. The accompanying figures are schematic and are not intended to be drawn to scale. For purposes of clarity, not every element or component is labeled in every figure, nor is every element or component of each embodiment of the inventive subject matter shown where illustration is not necessary to allow those of ordinary skill in the art to understand the inventive subject matter.

### Definitions

“Agent” as used herein refers to any treatment agent that can be administered to a patient for treatment and/or prevention of a disease and/or condition, including but not limited to a pharmaceutical agent, a drug, a small molecule drug, a drug conjugate, a prodrug, an antibody or an antibody fragment, a nucleic acid, a protein, a peptide, a polysaccharide, a small inorganic molecule, a small organic molecule (e.g., with a molecular weight of about 500 Da), a metabolically activated agent (e.g., a metabolite), a nutrient, a supplement, and the like, unless specified otherwise.

“Agent Drying Capacity Assay” as used herein refers to an assay used to determine a drying capacity for a drying agent in mg fluid absorbed/mg drying agent. According to the Agent Drying Capacity Assay, the drying capacity for the drying agent is measured according to a relative mass of a fluid absorbed by a predetermined mass of at a drying agent is measured at a temperature of 25° C. and 1 atmosphere of pressure. In particular, the relative mass of a 1× phosphate buffer solution (PBS) at 25° C. that is absorbed by a predetermined mass of a drying agent is determined, to evaluate the drying capacity of the drying agent. According to the Drying Capacity Assay, an empty 50 mL centrifuge tube is weighed and the initial mass is taken as a tare weight. 2 g of drying agent is added and the total mass of the tube and drying agent is taken as the pre-hydration mass. 40 mL of PBS is added to the tube and the tube is sealed. The tube is vortexed for 10 seconds to fully disperse drying agent into the PBS. The drying agent is allowed to soak in the PBS for 15 minutes. The tube is centrifuged at 2000 RPM for 15 minutes to separate the drying agent from the unabsorbed PBS. The resulting supernatant is decanted, and the mass of the soaked drying agent in the tube is taken. The mass of fluid absorbed by the drying agent is determined by subtracting the pre-soak mass from the post-soak mass. This value is divided by the initial mass of drying agent (e.g., 2 g) to determine the mass of fluid absorbed per mass of drying agent, in mg fluid/mg drying agent.

“Agent Fluid Uptake Assay” as used herein refers to an assay used to determine an extent of fluid that can be absorbed by a particular drying agent (fluid uptake capacity), as measured in mg of fluid taken up by a sample of the drying agent/mg of the drying agent sample. According to the Agent Fluid Uptake Assay, the fluid uptake for the drying agent of a pH 6.0 phosphate-buffered saline solution is measured at a temperature of 25° C. and 1 atmosphere of pressure. In particular, a pH 6.0 phosphate-buffered saline (PBS) media is prepared in a ratio by volume 100/10/0.675 of deionized water/10× concentrate PBS (25.6 g Na₂HPO₄.7H₂O+80 g NaCl+2 g KH₂PO₄, brought to 1 L with H₂O)/1N hydrochloric acid. 40 mL of the PBS media is weighed out, and the mass recorded. 500 g of a drying agent is weighed out as a drying agent sample, and the mass recorded. The drying agent sample is then immersed in the PBS media for 2 hours. The PBS media containing the drying agent sample is poured onto a polyester mesh disk filter (86×86 mesh, 4 9/16″ diameter, 0.0056″ opening) for 10 seconds, and the filter is allowed to drain an additional 60 seconds. The mass of the fluid drained through the filter is taken. A Media Uptake Ratio is calculated using the following formula:

MUR=F₀−Fᵣ/P

where MUR is the Media Uptake Ratio in mg fluid uptake/mg drying agent, F₀ is the initial mass of the fluid in mg before addition of the drying agent sample, Fᵣ is the mass of the fluid drained from the drying agent sample, and P is the initial mass of the drying agent sample. The fluid uptake capacity for the drying agent as measured by the Agent Fluid Uptake Assay is thus the MUR as determined for this 2 hour immersion time period.

“Agent Drying Time Assay” as used herein refers to an assay used to determine a time in which a predetermined extent of drying by a drying agent is provided. In the Agent Drying Time Assay, a relative mass of a fluid absorbed by a predetermined mass of a drying agent over a range of set time periods is measured at 25° C. and 1 atmosphere of pressure. In particular, the relative mass of a 1× phosphate buffer solution (PBS) at 25° C. that is absorbed by a mass of a drying agent over multiple set time periods is determined, to evaluate the drying time of the drying agent. According to the Agent Drying Time Assay, an empty 50 mL centrifuge tube is weighed and the initial mass is taken as the tare weight. 2 g of drying agent is added and the total mass of tube and drying agent is taken as the pre-hydration mass. 40 mL PBS is added to the tube and the tube is sealed. The tube is vortexed for 10 seconds to fully disperse drying agent into the PBS. The drying agent is allowed to soak in the PBS for 30 seconds. The tube is centrifuged at 2000 RPM for 5 min to separate the drying agent from unabsorbed PBS. The resulting supernatant is removed and the mass of the soaked drying agent in the tube is taken. The mass of fluid absorbed by the drying agent is determined by subtracting the pre-soak mass from the post-soak mass. This value is divided by the initial mass of drying agent (e.g. 2 g) to determine the mass of fluid absorbed per mass of drying agent, in mg fluid/mg drying agent. This procedure is repeated, changing the time in which the drying agent soaks in PBS to 1 minute, 5 minutes, 10 minutes and 15 minutes. The mg fluid absorbed/mg drying agent from all time points will be compared and the drying time is determined by selecting the earliest time point achieves at least 90% of the drying capacity as determined by the Agent Drying Capacity Assay for the same drying agent.

“Agent Fluid Uptake Time Assay” as used herein refers to an assay used to determine a time in which a predetermined uptake of fluid by a drying agent is provided. In the Agent Fluid Uptake Time Assay, the Agent Fluid Uptake Assay described above is performed to determine the media uptake ratio (MUR) at different time points. For example, the drying agent sample may be allowed to soak in the PBS media for a number of different time periods time periods ranging from 10 seconds to 2 hours, such as time periods of 1 minute, 5 minutes, 10 minutes, 20 minutes, 30 minutes, 40 minutes, 1 hour and 2 hours. The MURs from all time points are compared, and the time for fluid uptake according to the Agent Fluid Uptake Time Assay is determined by selecting the earliest time point that achieves 90% of the MUR that is established as the extent of the fluid that can be absorbed by the drying agent (the fluid uptake capacity) by the Agent Fluid Uptake Assay. If none of the evaluated time points achieve 90% of the fluid uptake capacity, i.e. if the evaluated time points exhibit MURs that are either below or above 90% of the fluid uptake capacity, then new time points are selected that are either above or below the evaluated time points, and the process of determining the MUR for the newly selected time points is repeated, until a time point is identified where the drying agent exhibits 90% of its fluid uptake capacity.

“Biodegradable” as used herein refers to materials that, when introduced into the body of an individual, patient, or subject, is broken down by cellular machinery or chemical processes (e.g., hydrolysis) into components (“degradation products”) that the body can either reuse or dispose of without significant toxic effect. In some instances, the degradation products may also be biocompatible.

“Dosage Form Drying Capacity Assay” as used herein refers to an assay used to determine a drying capacity for an oral dosage form in mg fluid absorbed/oral dosage form, as measured at 25° C. and 1 atmosphere of pressure. In the Drying Capacity Assay, an oral dosage form is crushed and/or pressed to provide particles having a size of no more than 1 mm. An empty 50 mL centrifuge tube is weighed and the initial mass is taken as a tare weight. The crushed/pressed dosage form is added and the total mass of the tube and oral dosage form is taken as the pre-hydration mass. 40 mL of PBS at 25° C. is added to the tube and the tube is sealed. The tube is vortexed for 10 seconds to fully disperse the oral dosage form into the PBS. The oral dosage form is allowed to soak in the PBS for 15 minutes. The tube is centrifuged at 2000 RPM for 15 minutes to separate the oral dosage form from the unabsorbed PBS. The resulting supernatant is decanted, and the mass of the soaked oral dosage form in the tube is taken. The mass of fluid absorbed by the oral dosage form is determined by subtracting the pre-soak mass from the post-soak mass. This value is the drying capacity in mass of fluid absorbed per oral dosage form, in mg fluid/oral dosage form.

“Dosage Form Fluid Uptake Assay” as used herein refers to an assay used to determine an extent of fluid that can be absorbed by a particular dosage from (fluid uptake capacity), as measured in mg of fluid taken up by a dosage form/mg of dosage form. According to the Dosage Form Fluid Uptake Assay, the fluid uptake for the dosage form of a pH 6.0 phosphate-buffered saline solution is measured at a temperature of 25° C. and 1 atmosphere of pressure. In particular, a pH 6.0 phosphate-buffered saline (PBS) media is prepared in a ratio by volume 100/10/0.675 of deionized water/10× concentrate PBS (25.6 g Na₂HPO₄.7H₂O+80 g NaCl+2 g KH₂PO₄, brought to 1 L with H₂O)/1N hydrochloric acid. 100 mL of the PBS media is weighed out, and the mass recorded. The oral dosage form is crushed and/or pressed to provide particles having a size of no more than 1 mm (e.g., 1 g of material). The dosage form particles are then immersed in the PBS media for 2 hours. The PBS media containing the dosage form particles is poured onto a polyester mesh disk filter (86×86 mesh, 4 9/16″ diameter, 0.0056″ opening) for 10 seconds, and the filter is allowed to drain an additional 60 seconds. The mass of the fluid drained through the filter is taken. A dosage form total Media Uptake is calculated using the following formula:

MU_{D}=F₀−Fᵣ

where MU_{D} is the dosage form total Media Uptake in mg fluid uptake per dosage form, F₀ is the initial mass of the fluid in mg before addition of the dosage form particles, and Fᵣ is the mass of the fluid drained from the dosage form particles. The fluid uptake capacity for the dosage form as measured by the Dosage Form Fluid Uptake Assay is thus the MU_{D} as determined for this 2 hour immersion time period.

“Dosage Form Fluid Uptake Time Assay at pH” as used herein refers to an assay used to determine a time in which a predetermined uptake of fluid by an entire dosage form is provided, when the dosage form is exposed to a pH at which a protective coating about the dosage form dissolves and/or becomes permeable. In the Dosage Form Fluid Uptake Time Assay at pH, a dosage form having a pH-dependent coating, such as an enteric coating, is immersed in a phosphate buffer solution (PBS) at 25° C. and 1 atmosphere of pressure, and the pH of the solution is adjusted to a predetermined pH at which the pH-dependent coating dissolves and/or becomes permeable (if not already at that pH at the point of immersion), such as a pH of at least 7.4. A relative mass of the fluid absorbed by the dosage form over a range of set time periods is measured at 25° C. and 1 atmosphere of pressure at the predetermined pH. In particular, the relative mass of a phosphate buffer solution (PBS) at 25° C. that is absorbed by the entire dosage form at the predetermined pH over multiple set time periods is determined, to evaluate the drying time of the dosage form. For example, the time periods at which the fluid uptake is evaluated may be time periods ranging from 10 seconds to 2 hours, such as time periods of 1 minute, 5 minutes, 10 minutes, 20 minutes, 30 minutes, 40 minutes, 1 hour and 2 hours. According to the Dosage Form Fluid Uptake Time Assay at pH, a pH 6.0 phosphate-buffered saline (PBS) media is prepared in a ratio by volume 100/10/0.675 of deionized water/10× concentrate PBS (25.6 g Na₂HPO₄.7H₂O+80 g NaCl+2 g KH₂PO₄, brought to 1 L with H₂O)/1N hydrochloric acid. 150 mL of the PBS media is weighed out, and the mass recorded. The dosage form is then immersed in the PBS media, and the pH is adjusted to pH 7.4 to at least partially dissolve and/or render permeable the enteric coating. The dosage form is allowed to soak in the PBS media for the predetermined time period, after which the PBS media containing the dosage form is poured onto a polyester mesh disk filter (86×86 mesh, 4 9/16″ diameter, 0.0056″ opening) for 10 seconds, and the filter is allowed to drain an additional 60 seconds. The mass of the fluid drained through the filter is taken. The total media uptake MU_{D} for the entire dosage form is calculated, as in the Dosage Form Fluid Uptake Assay above, to determine an extent of fluid uptake for the entire dosage form at the time point, as measured in mg fluid absorbed per dosage form. In particular, the MU_{D} is determined according to the following formula:

MU_{D}=F₀−Fᵣ

where MU_{D} is the total Media Uptake for the dosage form in mg fluid uptake per dosage form, F₀ is the initial mass of the fluid in mg before addition of the dosage form, and Fᵣ is the mass of the fluid drained from the dosage form. This procedure is repeated, changing the time in which the dosage form soaks in PBS. The mg fluid absorbed/dosage form from all time points will be compared and the fluid uptake time is determined by selecting the earliest time point that achieves 90% of the dosage form fluid uptake capacity as determined by the Dosage Form Fluid Uptake Assay for the same dosage form. If none of the evaluated time points achieve 90% of the fluid uptake capacity for the dosage form, i.e. if the evaluated time points exhibit MU_{D}s that are either below or above 90% of the fluid uptake capacity for the dosage form, then new time points are selected that are either above or below the evaluated time points, and the process of determining the MU_{D} for the newly selected time points is repeated, until a time point is identified where the dosage form exhibits 90% of its fluid uptake capacity.

“Dosage Form Fluid Uptake Time Assay for Uptake Phase” as used herein refers to an assay used to determine a time required for a phase of fluid uptake by the dosage form to occur, the fluid uptake phase occurring during a predetermined time in which the dosage form goes a first percentage of its fluid uptake capacity to a second percentage of its fluid uptake capacity, as measured by the Dosage Form Fluid Uptake Assay. For example, the fluid uptake phase may correspond to a time period in which the percentage of the fluid uptake of the dosage form achieved by absorption of the fluid increases by at least 50%, such as a time period required for the dosage form to absorb from 0% of its total possible fluid uptake capacity to 50% of its fluid uptake capacity, and/or a time period required for the dosage form to absorb from 1% to 51% of its fluid uptake capacity, and/or a time period required for the dosage form to absorb from 5% to 55% of its fluid uptake capacity, and/or a time period required for the dosage form to absorb from 10% to 60% of its fluid uptake capacity, and/or a time period required for the dosage form to absorb from 25% to 75% of its fluid uptake capacity, and/or a time period required for the dosage form to absorb from 50% to 100% of its fluid uptake capacity. In the Dosage Form Fluid Uptake Time for Drying Phase, a dosage form is immersed in phosphate buffer solution (PBS) at 25° C. and 1 atmosphere of pressure. A relative mass of the fluid absorbed by the dosage form over a range of set time periods is measured at 25° C. and 1 atmosphere of pressure, to determine the time it takes for the dosage form to absorb an amount of fluid in the predetermined drying phase. According to the Dosage Form Fluid Uptake Time Assay for Uptake Phase, a pH 6.0 phosphate-buffered saline (PBS) media is prepared in a ratio by volume 100/10/0.675 of deionized water/10× concentrate PBS (25.6 g Na₂HPO₄.7H₂O+80 g NaCl+2 g KH₂PO₄, brought to 1 L with H₂O)/1N hydrochloric acid. 150 mL of the PBS media is weighed out, and the mass recorded. The dosage form is then immersed in the PBS media, and allowed to soak at least until the dosage form begins to at least partially dissolve in the media. For example, a time until the dosage form begins to dissolve in the fluid media may be in the range of from 10 seconds to up 8 or 24 hours or longer, depending on the composition of the dosage form. Once the dosage form begins to at least partially dissolve in the fluid media, immersion of the dosage form in the fluid media is continued for a predetermined period of time. For example, the predetermined period of time after the dosage form has begun to at least partially dissolve may be a time period in the range of from 10 seconds to 2 hours, such as time periods of 1 minute, 5 minutes, 10 minutes, 20 minutes, 30 minutes, 40 minutes, 1 hour and 2 hours. After the predetermined period of time has passed, the PBS media containing the dosage form is poured onto a polyester mesh disk filter (86×86 mesh, 4 9/16″ diameter, 0.0056″ opening) for 10 seconds, and the filter is allowed to drain an additional 60 seconds. The mass of the fluid drained through the filter is taken. The total media uptake MU_{D} for the entire dosage form is calculated, as in the Dosage Form Fluid Uptake Assay above, to determine an extent of fluid uptake for the entire dosage form at the time point, as measured in mg fluid absorbed/mg dosage form. In particular, the MU_{D} is determined according to the following formula:

MU_{D}=F₀−Fᵣ

where MU_{D} is the total Media Uptake in mg fluid uptake per dosage form, F₀ is the initial mass of the fluid in mg before addition of the dosage form, and Fᵣ is the mass of the fluid drained from the dosage form. This procedure is repeated, changing the time in which the dosage form soaks in the PBS fluid. The mg fluid absorbed/dosage form from all time points are compared to determine one or more fluid uptake phases where at least a 50% increase in the amount of fluid absorbed by the dosage form is achieved (e.g., a phase where a change in the fluid uptake extent is from 25% to 75%). The fluid uptake time for the one or more fluid uptake phases, such as a phase that achieves at least a 50% increase in the amount of fluid absorbed (e.g., from a fluid uptake extent of 25% to a fluid uptake extent of 75%), is then determined by taking a difference between the time points representing the end points of the phase. For example, the fluid uptake time may be the difference between the time point at which 25% of the fluid uptake capacity is achieved, and the time point at which 75% of the fluid uptake capacity is achieved, the fluid uptake capacity being determined by the Dosage Form Fluid Uptake Assay for the same dosage form. Accordingly, a time required for a fluid uptake phase in which the dosage form increases fluid uptake by at least 50% can be determined.

“Dosage Form Fluid Uptake Time Assay at Breakthrough” as used herein refers to an assay used to determine a time in which a predetermined extent of fluid uptake by an entire dosage form is crushed and/or pressed into particles, so that contents of the dosage form that would otherwise be covered by a protective coating can be exposed to fluid. In the Dosage Form Fluid Uptake Time Assay at Breakthrough, The dosage form is crushed and/or pressed to provide particles having a size of no more than 1 mm. In the Dosage Form Fluid Uptake Time at Breakthrough, the dosage form particles are immersed in a phosphate buffer solution (PBS) at 25° C. and 1 atmosphere of pressure. A relative mass of the fluid absorbed by the dosage form particles over a range of set time periods is measured at 25° C. and 1 atmosphere of pressure, to determine the time it takes for the dosage form particles to absorb an amount of fluid. For example, the time periods at which the fluid uptake is evaluated may be time periods ranging from 10 seconds to 2 hours, such as time periods of 1 minute, 5 minutes, 10 minutes, 20 minutes, 30 minutes, 40 minutes, 1 hour and 2 hours. According to the Dosage Form Fluid Uptake Time Assay at Breakthrough, a pH 6.0 phosphate-buffered saline (PBS) media is prepared in a ratio by volume 100/10/0.675 of deionized water/10× concentrate PBS (25.6 g Na₂HPO₄.7H₂O+80 g NaCl+2 g KH₂PO₄, brought to 1 L with H₂O)/1N hydrochloric acid. 150 mL of the PBS media is weighed out, and the mass recorded. The dosage form particles are then immersed in the PBS media. The dosage form particles allowed to soak in the PBS media for the predetermined time period, after which the PBS media containing the dosage form particles is poured onto a polyester mesh disk filter (86×86 mesh, 4 9/16″ diameter, 0.0056″ opening) for 10 seconds, and the filter is allowed to drain an additional 60 seconds. The mass of the fluid drained through the filter is taken. The media uptake ratio MU_{D} for the entire dosage form is calculated, as in the Dosage Form Fluid Uptake Assay above, to determine an extent of fluid uptake for the dosage form particles at the time point, as measured in mg fluid absorbed/dosage form. In particular, the MU_{D} is determined according to the following formula:

MU_{D}=F₀−Fᵣ

where MU_{D} is the total Media Uptake for the dosage form in mg fluid uptake per dosage form, F₀ is the initial mass of the fluid in mg before addition of the dosage form particles, and Fᵣ is the mass of the fluid drained from the divided dosage form. This procedure is repeated, changing the time in which the dosage form particles soak in PBS. The mg fluid absorbed/dosage form from all time points will be compared and the fluid uptake time is determined by selecting the earliest time point that achieves 90% of the dosage form fluid uptake capacity as determined by the Dosage Form Fluid Uptake Assay for the same dosage form. If none of the evaluated time points achieve 90% of the fluid uptake capacity for the dosage form, i.e. if the evaluated time points exhibit MU_{D}s that are either below or above 90% of the fluid uptake capacity for the dosage form, then new time points are selected that are either above or below the evaluated time points, and the process of determining the MU_{D} for the newly selected time points is repeated, until a time point is identified where the dosage form exhibits 90% of its fluid uptake capacity.

“Endoscopic Bioavailability Assay” as used herein refers to an assay used to determine bioavailability of an active agent using an endoscopic testing method. In the Endoscopic Bioavailability Assay, a porcine model is used, where the oral dosage form is inserted via endoscope into the animal's mouth and advanced to the region of the intestine of interest, such as the duodenum or ileum, where the oral dosage form is released. Blood samples are collected after placement of the oral dosage form, and the area under the curve (AUC) is computed. The same animal is given a subcutaneous injection at a therapeutic dose, blood samples are collected at the same time intervals, and the area under the curve (AUC) is calculated. The percent bioavailability is calculated as a dose normalized ratio of the AUC of the oral dosage form divided by the AUC for the subcutaneous injection.

“Individual,” “patient,” or “subject” as used herein are used interchangeably and refer to any animal, including mammals, preferably mice, rats, guinea pigs, and other rodents; rabbits; dogs; cats; swine; cattle; sheep; horses; birds; reptiles; or primates, such as humans.

“Mucoadhesive” as used herein refers to a composition having the capacity to bind to a mucosal surface.

“Pharmaceutically acceptable carrier” or “pharmaceutically acceptable excipient” as used herein refers to any and all solvents, dispersion media, coatings, isotonic and absorption delaying agents, and the like, that are biocompatible and otherwise suitable for administration to an Individual.

“Pharmaceutical composition” as used herein refers to a composition comprising at least one agent as disclosed herein formulated together with one or more pharmaceutically acceptable carriers and/or excipients.

“Pharmaceutically or pharmacologically acceptable” as used herein refers to molecular entities and compositions that are acceptable for administration to an animal, or a human, as appropriate, for example in not producing an excessive adverse, allergic, or other untoward reaction.

“Port Bioavailability Assay” as used herein refers to an assay used to determine bioavailability of an active agent using a port testing method. In the Port Bioavailability Assay, Yucatan Minipigs are used, and a medical grade jejunal tube is placed via laparotomy into the small intestine, reaching the duodenum and even the ileum. A venous access port is provided for blood collection. To test the bioavailability resulting from dosage forms, the dosage forms are placed into the small intestine through the tube, using forceps. Optionally, the interaction of the dosage form with the intestinal environment can be observed via endoscopy through the port. The animal is placed on a fasting diet for 16 hours prior to the dosage form insertion. Blood collection to assess bioavailability includes 7 draws, with one draw taking place before dosage form insertion, and subsequent draws at 30 mins., 60 mins., 1.5 hours, 2, hours, 3, hours, 4 hours, 5 hour and 6 hours after dosage form insertion. The percent bioavailability of the active agent provided with the oral dosage form is determined on the basis of the active agent levels detected in these blood samples.

“Surgical Bioavailability Assay” as used herein refers to an assay used to determine bioavailability of an active agent using a surgical testing method. In the Surgical Bioavailability Assay, a porcine model is also used, in which the animals undergo open surgery such that oral dosage forms to be tested can be placed at regions of the intestine of interest, such as the jejunum and ileum. After administration of anesthesia, a ventral midline incision is made to gain access to the animal's abdominal cavity. The jejunum and ileum are exposed, and incisions are made to manually place the oral dosage forms being tested. After oral dosage form placement is complete, the intestinal incisions are closed, and the midline incision can also be temporarily closed. Also, access can be made for blood collection via the carotid artery or jugular vein. The animal can be kept under anesthesia for up to 4 hours to allow for blood collection, such as at 5 mins, 15 mins, 30 mins, 60 mins, 90 mins, and 2 hours after oral dosage form placement. Blood samples can also be collected prior to the surgical procedure. The percent bioavailability of the active agent provided with the oral dosage form is determined on the basis of the active agent levels detected in these blood samples.

“Treating” as used herein refers to any effect, for example, lessening, reducing or modulating, that results in the improvement of the condition, disease, disorder, and the like.

The singular forms “a,” “an,” and “the,” as used herein, include plural referents unless the context clearly dictates otherwise.

The terms “comprising,” “comprises,” “including,” and “includes” should be interpreted as referring to elements, components, or steps in a non-exclusive manner, indicating that the referenced elements, components, or steps may be present, utilized, or combined with other elements, components, or steps that are not expressly referenced.

### DETAILED DESCRIPTION

Aspects of the present disclosure are directed to dosage forms, systems and methods for the oral, trans-intestinal, and/or trans-mucosal delivery of an active agent. In particular, aspects of the present disclosure relate to an oral dosage form having a drying composition with one or more drying agents in a formulation that enhances the bioavailability of an active agent being delivered therewith. According to one embodiment, the enhanced bioavailability imparted by the oral dosage form can result in improved delivery of active agents for the treatment of medical conditions, thereby enhancing the overall efficacy of the treatment. In particular, the oral dosage form may be capable in one embodiment of enhancing the bioavailability of polypeptides and other high-molecular weight small molecules that otherwise exhibit poor absorption in the gastrointestinal tract. This enhancement may permit oral dosing in some individuals who would otherwise have been non-compliant with subcutaneous injections. In another embodiment, the increased bioavailability achieved with the oral dosage form may reduce the amount of active agent that is required to be included in the oral dosage form to achieve a therapeutic treatment effect. Aspects of the disclosure further relate to methods and systems for delivery of an active agent using the oral dosage form, as well as methods of preparing the oral dosage forms described herein.

Without being limited to any one particular theory, in one embodiment, it is believed that enhanced bioavailability of the active agent is achieved by providing an oral dosage form having at least one drying agent and/or a combination of multiple drying agents as a part of a drying composition that imparts a drying effect at a site of the intestinal tissue where delivery of the active agent occurs. In particular, in one embodiment, the oral dosage form is capable of delivering the active agent in combination with a drying composition to an intestinal tissue site, where release of the drying composition from the oral dosage form provides a localized drying effect to an area about an intestinal site to which the active agent is being delivered. The localized drying effect in the area about the intestinal site can result in a marked increase in the bioavailability of the active agent that is included with the drying composition as a part of the oral dosage form. Without being limited to any particular theory, it is believed that one mechanism by which localized drying of an area about the intestinal delivery site increases bioavailability of the active agent, may be in increasing the effective local concentration of the active agent, providing a greater driving force to transport the active agent across the intestinal wall. Similarly, without being limited to a particular theory, a mechanism for the enhanced bioavailability exhibited in conjunction with the localized drying effect may be as a result of the increase in the permeability of the intestinal tissue, driven by the influx of fluid from the intestinal wall to hydrate the drying agent and active agent. Other mechanisms by which the localized drying effect provides enhanced bioavailability, and which are as-yet unknown, may also exist.

In one embodiment, the drying effect at the site of delivery of the active agent to the intestine may be achieved by providing an oral dosage form that is formulated with a drying composition having at least one drying agent in an amount, composition and/or configuration that dries the local region about the intestinal tissue site to improve the bioavailability of the active agent. For example, in one embodiment the drying composition may include a drying agent that is provided in a percent by weight that is sufficiently high to achieve a drying effect at delivery site of the intestinal tissue. In another embodiment, the drying composition comprises a drying agent that exhibits a high absorption capacity, as measured by a Drying Capacity Assay and/or a Fluid Uptake Assay. The configuration of the oral dosage form in one embodiment can also be devised such that the drying effect is achieved before and/or simultaneously with release of the active agent from the oral dosage form, for example by providing different layers or configurations of the oral dosage form. Furthermore, additional agents that can increase the delivery of the active agent to and/or across the intestinal epithelium can also be provided as a part of the oral dosage form, such as for example at least one of gelling agents, permeation enhancers, osmagents, and even mucoadhesive agents can be included as a part of the oral dosage form. Detailed discussion of embodiments of the oral dosage form that are capable of imparting the local drying effect to enhance active agent absorption and bioavailability is provided below.

Target Tissue

In one embodiment, the oral dosage form is configured to provide delivery of the active agent and/or drying composition to a target tissue within the gastrointestinal tract, such as for example the upper gastrointestinal tract or the lower gastrointestinal tract (i.e., the small intestine or large intestine). For example, in one embodiment, the site of delivery of the active agent may be to the mucosa of the small intestine (e.g., the duodenum, jejunum, or ileum) and/or the large intestine (e.g., the ascending colon, the right colic flexure, the transverse colon, the transverse mesocolon, the left colic flexure, the descending colon, the sigmoid colon, and the rectum). In one embodiment, the oral dosage form is configured to provide delivery of the active agent and drying composition to tissue in the ileum of the small intestine.

According to one embodiment, delivery to a particular region of the gastrointestinal tract, such as to a site in the small intestine, can be achieved by selecting the configuration and composition of the oral dosage form. For example, an protective coating such as an enteric coating can be provided that at least partially shields the dosage form during transit through the stomach and/or other areas of the upper gastrointestinal tract, until a predetermined location in the lower gastrointestinal tract is reached. Further discussion of embodiments of a protectively coated and/or enterically coated dosage form and/or other forms capable of delivering an active agent to a predetermined location in the gastrointestinal tract is provided in further detail below.

In one embodiment, by providing an oral dosage form that targets the ileum of the intestine, enhanced bioavailability can be provided. Targeting of the ileum may be achieved, in one embodiment, by providing a protective and/or enteric coating that is specifically configured to provide release of an active agent from the oral dosage form, once the dosage form has traveled through the gastrointestinal tract to reach the ileum. While the intestine is understood to become generally drier in moving from the duodenum to the jejunum to the ileum, certain active agents injected in the form of a solution into these regions of the intestine nonetheless show worse uptake in the ileum than in the jejunum or duodenum. That is, certain active agents injected in solution form exhibit higher uptake in regions of the intestine that are less dry, such as the jejunum or duodenum. Thus, the enhanced bioavailability that can be provided by the drying effect of the oral dosage form, according to aspects of the present disclosure, is counter-intuitive in light of the reduced uptake exhibited by solution forms of active agents that are injected into a drier region in the intestine.

Active Agent

The oral dosage form according to embodiments of the present disclosure is adapted to deliver any of a wide range of active agents to a tissue site. Thus, for example, the oral dosage form may be adapted to deliver a single active agent or multiple active agents (e.g., two, three or more active agents, either serially or simultaneously) to the tissue site. Additionally, the active agents may be in any of a wide range of alternative forms such as pharmaceutically acceptable salt forms, free acid forms, free base forms, and hydrates.

In general, the active agent may be in particulate, liquid, or gel form and may comprise any of a range of compositions having biological relevance, e.g., metals, metal oxides, peptides, peptides structurally engineered to resist enzymatic degradation, antibodies, hormones, enzymes, growth factors, small organic molecules, ligands, or other pharmaceuticals, nutraceuticals, or biologics. In some embodiments, the agent(s) may include one or more large molecules (e.g., proteins and/or protein conjugates), and/or one or more small molecules (e.g., small organic or inorganic molecules, and/or small peptides) as the agent(s). In one exemplary embodiment, the active agent comprises at least one polypeptide and/or small molecule having a therapeutic treatment effect. Examples of active agents that can be delivered by the oral dosage form can include at least one of octreotide, calcitonin (including salmon calcitonin), parathyroid hormone (PTH), teriparatide (a recombinant form of PTH) insulin, peptide agonists of GLP-1, such as exenatide, liraglutide, lixisenatide, albiglutide and/or dulaglutide, GLP-1/GIP co-agonists, GLP-2 agonists and peptide GPCR agonists.

In yet another embodiment, the active agent can comprise other large molecules and/or other structures other than those specifically listed above, such as for example any one or more of antibodies (monoclonal and polyclonal) or antibody fragments, polysaccharides, carbohydrates, nanoparticles, vaccines, biologics, nucleic acids, cells and cell therapies, DNA, RNA, siRNA, blood factors, gene therapies, thrombolytic agents (tissue plasminogen activator), growth factors (erythropoietin), interferons, interleukin-based molecules, fusion proteins, recombinant proteins, therapeutic enzymes, and others. The active agent may also and/or alternatively comprise at least one of a small molecule drug, a drug conjugate, a prodrug, a small inorganic molecule, a small organic molecule (e.g., with a molecular weight of about 500 Da), a metabolically activated agent (e.g., a metabolite), a nutrient, a supplement, and the like.

According to one embodiment, the oral dosage form is capable of providing good bioavailability in delivering an active agent that may be otherwise poorly absorbed in the intestine. For example, the oral dosage form having the drying composition can be capable of providing surprisingly good bioavailability for polypeptides and/or other small molecules having a relatively high molecular weight, which agents may be otherwise difficult to effectively administer due to their relatively large size. Examples of such active agents may include polypeptides and/or small molecules having a size of at least about 450 Da. However, according to one embodiment, the molecular weight of the active agent may still be below about 200,000 Da, to allow for good delivery/absorption of the active agent in the intestine. According to one example, in one embodiment the active agent has a molecular weight of at least about 2000 Da. By way of further example, in one embodiment the active agent has a molecular weight of at least about 5000 Da. By way of yet a further example, in one embodiment the active agent has a molecular weight of at least about 10,000 Da. While the active agent according to one embodiment will generally have a molecular weight below about 600,000 Da, as has been described above, the molecular weight may also in one example be below about 200,000 Da, such as below about 100,000 Da. For example, the active agent provided as a part of the oral dosage form may have a molecular weight in one embodiment that is in the range of from about 450 Da to about 500,000 Da, such as about 450 Da to about 25,000 Da, and even 450 Da to 10,000 Da, such as about 450 Da to about 6000 Da. For example, in one embodiment the active agent may have a molecular weight in a range of from about 1000 Da to about 25,000 Da, and even about 1,000 Da to about 10,000 Da, such as about 1000 Da to 5000 Da. As previously noted, the oral dosage form may contain two or more agents independently selected from molecules having a molecular weight within the ranges recited herein.

The oral dosage form comprises the at least one active agent in an amount or concentration that is suitable for the delivery of the active agent. For example, in one embodiment, a total content of the active agent in the dosage form may be at least about 0.0001% of the weight of the oral dosage form. By way of further example, in one embodiment, a total content of the active agent may be at least about 0.001% of the weight of the oral dosage form. By way of further example, in one embodiment, a total content of the active agent may be at least about 0.01% of the weight of the oral dosage form. By way of further example, in one embodiment, the active agent may be at least about 0.1% of the weight of the oral dosage form. By way of further example, in one embodiment, the active agent may be at least about 1% of the weight of the oral dosage form. By way of further example, in one embodiment, the active agent may be at least about 10% of the weight of the oral dosage form. By way of further example, in one embodiment, the active agent may be at least about 20% of the weight of the oral dosage form. By way of further example, in one embodiment, the active agent may be at least about 50% of the weight of the oral dosage form. By way of further example, in one embodiment the active agent is less than about 90% by weight of the oral dosage form. By way of further example, in one embodiment the active agent is less than about 25% by weight of the oral dosage form. By way of further example, in one embodiment the active agent is less than about 10% by weight of the oral dosage form. By way of further example, in one embodiment the active agent is less than about 5% by weight of the oral dosage form. In certain embodiments, the active agent may be between about 0.0001% and about 90% of the weight of the oral dosage form. By way of further example, in one embodiment, the active agent may be between about 0.01% and about 25% of the weight of the oral dosage form. By way of further example, in one embodiment, the active agent may be between about 1% and about 25% of the weight of the oral dosage form.

The content of the active agent in the oral dosage form can be selected according to the intended dose of the active agent to be provided, as well as the activity of the active agent. For example, in one embodiment, an active agent corresponding to octreotide may be provided in a content of at least about 0.3% of the weight of the oral dosage form. By way of further example, in one embodiment, the octreotide may be at least about 2.5% of the weight of the oral dosage form. By way of further example, in one embodiment, the octreotide may be at least about 5% of the weight of the oral dosage form. By way of further example, in one embodiment, the octreotide may be at least about 10% of the weight of the oral dosage form. In one embodiment the octreotide is provided in an amount of less than about 50% of the weight of the oral dosage form. By way of further example, in one embodiment the octreotide is less than about 25% of the weight of the oral dosage form. By way of further example, in one embodiment the octreotide is less than about 10% by weight of the oral dosage form. By way of further example, in one embodiment the octreotide is less than about 5% by weight of the oral dosage form. In certain embodiments, the octreotide may be between about 0.5% and about 50% of the weight of the oral dosage form. By way of further example, in one embodiment, the octreotide may be between about 2.5% and about 25% of the weight of the oral dosage form. By way of further example, in one embodiment, the octreotide may be between about 2.5% and about 10% of the weight of the oral dosage form.

In yet another embodiment, an active agent corresponding to calcitonin may be provided in a content of at least about 0.3% by weight of the oral dosage form. By way of further example, in one embodiment, the calcitonin may be at least about 2.5% of the weight of the oral dosage form. By way of further example, in one embodiment, the calcitonin may be at least about 5% of the weight of the oral dosage form. By way of further example, in one embodiment, the calcitonin may be at least about 10% of the weight of the oral dosage form. By way of further example, in one embodiment the calcitonin is less than about 50% by weight of the oral dosage form. By way of further example, in one embodiment the calcitonin is less than about 25% by weight of the oral dosage form. By way of further example, in one embodiment the calcitonin is less than about 10% by weight of the oral dosage form. By way of further example, in one embodiment the calcitonin is less than about 5% by weight of the oral dosage form. In certain embodiments, the calcitonin may be between about 0.5% and about 50% of the weight of the oral dosage form. By way of further example, in one embodiment, the calcitonin may be between about 2.5% and about 25% of the weight of the oral dosage form. By way of further example, in one embodiment, the calcitonin may be between about 2.5% and about 10% of the weight of the oral dosage form.

In another embodiment, an active agent corresponding to teriparatide may be provided in a content of at least about 0.3% by weight of the oral dosage form. By way of further example, in one embodiment, the teriparatide may be at least about 2.5% of the weight of the oral dosage form. By way of further example, in one embodiment, the teriparatide may be at least about 5% of the weight of the oral dosage form. By way of further example, in one embodiment, the teriparatide may be at least about 10% of the weight of the oral dosage form. By way of further example, in one embodiment the teriparatide is less than about 50% by weight of the oral dosage form. By way of further example, in one embodiment the teriparatide is less than about 25% by weight of the oral dosage form. By way of further example, in one embodiment the teriparatide is less than about 10% by weight of the oral dosage form. By way of further example, in one embodiment the teriparatide is less than about 5% by weight of the oral dosage form. In certain embodiments, the teriparatide may be between about 0.5% and about 50% of the weight of the oral dosage form. By way of further example, in one embodiment, the teriparatide may be between about 2.5% and about 25% of the weight of the oral dosage form. By way of further example, in one embodiment, the teriparatide may be between about 2.5% and about 10% of the weight of the oral dosage form.

In another embodiment, an active agent corresponding to exenatide may be provided in a content of at least about 0.001% by weight of the oral dosage form. By way of further example, in one embodiment, the exenatide may be at least about 0.01% of the weight of the oral dosage form. By way of further example, in one embodiment, the exenatide may be at least about 0.1% of the weight of the oral dosage form. By way of further example, in one embodiment, the exenatide may be at least about 1% of the weight of the oral dosage form. By way of further example, in one embodiment the exenatide is less than about 10% by weight of the oral dosage form. By way of further example, in one embodiment the exenatide is less than about 1% by weight of the oral dosage form. By way of further example, in one embodiment the exenatide is less than about 0.1% by weight of the oral dosage form. By way of further example, in one embodiment the exenatide is less than about 0.01% by weight of the oral dosage form. In certain embodiments, the exenatide may be between about 0.001% and about 10% of the weight of the oral dosage form. By way of further example, in one embodiment, the exenatide may be between about 0.01% and about 1% of the weight of the oral dosage form. By way of further example, in one embodiment, the exenatide may be between about 0.01% and about 0.1% of the weight of the oral dosage form.

In yet another embodiment, an active agent corresponding to liraglutide may be provided in a content of at least about 0.3% by weight of the oral dosage form. By way of further example, in one embodiment, the liraglutide may be at least about 2.5% of the weight of the oral dosage form. By way of further example, in one embodiment, the liraglutide may be at least about 5% of the weight of the oral dosage form. By way of further example, in one embodiment, the liraglutide may be at least about 10% of the weight of the oral dosage form. By way of further example, in one embodiment the liraglutide is less than about 50% by weight of the oral dosage form. By way of further example, in one embodiment the liraglutide is less than about 25% by weight of the oral dosage form. By way of further example, in one embodiment the liraglutide is less than about 10% by weight of the oral dosage form. By way of further example, in one embodiment the liraglutide is less than about 5% by weight of the oral dosage form. In certain embodiments, the liraglutide may be between about 0.5% and about 50% of the weight of the oral dosage form. By way of further example, in one embodiment, the liraglutide may be between about 2.5% and about 25% of the weight of the oral dosage form. By way of further example, in one embodiment, the liraglutide may be between about 2.5% and about 10% of the weight of the oral dosage form.

Drying Agent

As discussed above, in one embodiment the oral dosage form comprises a drying composition with at least one drying agent that is capable of providing a localized drying effect in an area of the intestine. For example, the drying composition may provide a content, configuration and/or amount of one or more drying agents that imparts the drying effect at a site of the intestine where the active agent is delivered, to enhance the bioavailability of the active agent, as is described in more detail in the embodiments below.

According to one embodiment, the drying composition has at least one drying agent that is provided in a content as a percent by weight that is relatively high with respect to the mass of the overall oral dosage form. For example, in one embodiment, the drying composition comprises at least one drying agent in a content of at least about 15% by weight of the oral dosage form. By way of further example, in one embodiment, the drying composition comprises at least one drying agent in a content of at least about 20% by weight of the oral dosage form. By way of further example, in one embodiment, the drying composition comprises at least one drying agent in a content of at least about 30% by weight of the oral dosage form. By way of further example, in one embodiment, the drying composition comprises at least one drying agent in a content of at least about 35% by weight of the oral dosage form. By way of further example, in one embodiment, the drying composition comprises at least one drying agent in a content of at least about 50% by weight of the oral dosage form. By way of further example, in one embodiment, the drying composition comprises at least one drying agent in a content of at least about 65% by weight of the oral dosage form. By way of yet further example, in one embodiment, the drying composition comprises at least one drying agent in a content of at least about 75% by weight of the oral dosage form. By way of yet further example, in one embodiment, the drying composition comprises at least one drying agent in a content of at least about 85% by weight of the oral dosage form. By way of yet further example, in one embodiment, the drying composition comprises at least one drying agent in a content of at least about 95% by weight of the oral dosage form. While the drying composition according to one embodiment will generally have a content of the at least one drying agent of less than about 99% by weight in the composition, the drying composition may also have a content of the at least one drying agent of less than about 75% by weight, such as less than about 50% by weight, and even less than about 35% by weight. For example, the drying composition in one embodiment may comprise the at least one drying agent in a content of from about 15% by weight to about 60% by weight of the oral dosage form, such as from about 20% by weight to about 40% by weight of the oral dosage form.

Furthermore, in one embodiment the weight percent of the at least one drying agent as referred to herein may correspond to a total weight percent of all drying agents in the dosage form. For example, for a dosage form containing only a single drying agent, the weight percent of the at least one drying agent in the form is the weight percent of just that single drying agent in the form (i.e., the weight percent of the single drying agent is the total drying agent content). As another example, for a dosage form containing two or more drying agents, the weight percent of the at least one drying agent is the total weight percent of all drying agents provided in the dosage form (i.e., the combined weight percents of the two or more drying agents is the total drying agent content). That is, in one embodiment, the dosage form has a total drying agent content of at least about 15% by weight. By way of further example, in one embodiment, the dosage form has a total drying agent content of at least about 20% by weight. By way of further example, the dosage form has a total drying agent content of at least about 30% by weight. By way of further example, in one embodiment, the dosage form has a total drying agent content of at least about 35% by weight. By way of further example, in one embodiment, the dosage form has a total drying agent content of at least about 50% by weight. By way of further example, the dosage form has a total drying agent content of at least about 65% by weight of the dosage form. By way of yet further example, in one embodiment, the dosage form has a total drying agent content of at least about 75% by weight. By way of yet further example, the dosage form has a total drying agent content of least about 85% by weight. By way of yet further example, in one embodiment, the dosage form has a total drying agent content of at least about 95% by weight. While the dosage form according to one embodiment will generally have total drying agent content of less than about 99% by weight, the total drying agent content may also be less than about 75% by weight, such as less than about 50% by weight, and even less than about 35% by weight. For example, the total drying agent content in one embodiment may be from about 15% by weight to about 60% by weight of the dosage form, such as from about 20% by weight to about 40% by weight of the dosage form.

In one embodiment, the at least one drying agent is selected according to a drying capacity of the agent, which can be determined according to an assay such as the Agent Drying Capacity Assay described herein. In the Agent Drying Capacity Assay, a relative mass of a fluid absorbed by a predetermined mass of at a drying agent is measured at 25° C. and 1 atmosphere of pressure. In particular, the relative mass of a 1× phosphate buffer solution (PBS) at 25° C. that is absorbed by a predetermined mass of a drying agent is determined, to evaluate the drying capacity of the drying agent. According to the Drying Capacity Assay, an empty 50 mL centrifuge tube is weighed and the initial mass is taken as a tare weight. 2 g of drying agent is added and the total mass of the tube and drying agent is taken as the pre-hydration mass. 40 mL of PBS is added to the tube and the tube is sealed. The tube is vortexed for 10 seconds to fully disperse drying agent into the PBS. The drying agent is allowed to soak in the PBS for 15 minutes. The tube is centrifuged at 2000 RPM for 15 minutes to separate the drying agent from the unabsorbed PBS. The resulting supernatant is decanted, and the mass of the soaked drying agent in the tube is taken. The mass of fluid absorbed by the drying agent is determined by subtracting the pre-soak mass from the post-soak mass. This value is divided by the initial mass of drying agent (e.g., 2 g) to determine the mass of fluid absorbed per mass of drying agent, in mg fluid/mg drying agent. According to one embodiment, the drying capacity as measured in the mg PBS fluid absorbed per mg of drying agent in the Agent Drying Capacity Assay is at least about 1 mg fluid/mg drying agent. By way of further example, in one embodiment the drying capacity of the drying agent as measured by the Agent Drying Capacity Assay is at least about 3 mg fluid/mg drying agent. By way of further example, in one embodiment the drying capacity of the drying agent as measured by the Agent Drying Capacity Assay is at least about 4 mg fluid/mg drying agent. By way of further example, in one embodiment the drying capacity of the drying agent as measured by the Agent Drying Capacity Assay is at least about 5 mg fluid/mg drying agent. By way of further example, in one embodiment the drying capacity of the drying agent as measured by the Agent Drying Capacity Assay is at least about 6 mg fluid/mg drying agent. By way of even further example, in one embodiment the drying capacity of the drying agent as measured by the Agent Drying Capacity Assay is at least about 7 mg fluid/mg drying agent. By way of further example, in one embodiment the drying capacity of the drying agent as measured by the Agent Drying Capacity Assay is at least about 10 mg fluid/mg drying agent. By way of further example, in one embodiment the drying capacity of the drying agent as measured by the Agent Drying Capacity Assay is at least about 20 mg fluid/mg drying agent. In general, the drying capacity of the drying agent as measured by the Agent Drying Capacity Assay will be less than about 40 mg fluid/mg drying agent, and may even be less than about 15 mg fluid/mg drying agent. In one embodiment, the drying capacity of the drying agent as measured by the Agent Drying Capacity Assay is from about 1 mg fluid/mg drying agent to about 20 mg fluid/mg drying agent, such as from about 3 mg fluid/mg drying agent to about 20 mg fluid/mg drying agent, and even from about 5 mg fluid/mg drying agent to about 20 mg fluid/mg drying agent.

In one embodiment, the at least one drying agent is selected according to a drying time of the agent, as determined according to an Agent Drying Time Assay described herein. In the Agent Drying Time Assay, a relative mass of a fluid absorbed by a predetermined mass of a drying agent over a range of set time periods is measured at 25° C. and 1 atmosphere of pressure. In particular, the relative mass of a 1× phosphate buffer solution (PBS) at 25° C. that is absorbed by a mass of a drying agent over multiple set time periods is determined, to evaluate the drying time of the drying agent. According to the Agent Drying Time Assay, an empty 50 mL centrifuge tube is weighed and the initial mass is taken as the tare weight. 2 g of drying agent is added and the total mass of tube and drying agent is taken as the pre-hydration mass. 40 mL PBS is added to the tube and the tube is sealed. The tube is vortexed for 10 seconds to fully disperse drying agent into the PBS. The drying agent is allowed to soak in the PBS for 30 seconds. The tube is centrifuged at 2000 RPM for 5 min to separate the drying agent from unabsorbed PBS. The resulting supernatant is removed and the mass of the soaked drying agent in the tube is taken. The mass of fluid absorbed by the drying agent is determined by subtracting the pre-soak mass from the post-soak mass. This value is divided by the initial mass of drying agent (e.g. 2 g) to determine the mass of fluid absorbed per mass of drying agent, in mg fluid/mg drying agent. This procedure is repeated, changing the time in which the drying agent soaks in PBS to 1 minute, 5 minutes, 10 minutes and 15 minutes. The mg fluid absorbed/mg drying agent from all time points will be compared and the drying time is determined by selecting the earliest time point achieves at least 90% of the drying capacity as determined by the Agent Drying Capacity Assay for the same drying agent, in seconds. According to one embodiment, the drying time as measured in seconds in the Agent Drying Time Assay is no more than about 5 seconds. By way of further example, in one embodiment the drying time of the drying agent as measured by the Agent Drying Time Assay is no more than about 30 seconds. By way of even further example, in one embodiment the drying time of the drying agent as measured by the Agent Drying Time Assay is no more than about 60 seconds. By way of even further example, in one embodiment the drying time of the drying agent as measured by the Agent Drying Time Assay is no more than about 300 seconds. By way of even further example, in one embodiment the drying time of the drying agent as measured by the Agent Drying Time Assay is no more than about 600 seconds. By way of even further example, in one embodiment the drying time of the drying agent as measured by the Agent Drying Time Assay is no more than about 900 seconds. In general, the drying time of the drying agent as measured by the Agent Drying Time Assay will be less that about 1800 seconds. In one embodiment, the drying time of the drying agent as measured by the Agent Drying Time Assay is from about 5 seconds to about 1800 seconds, such as from about 30 seconds to about 900 seconds, and even from about 300 seconds to about 600 seconds.

In one embodiment, the composition and content of drying composition of the oral dosage form is selected to provide a drying capacity for the entire oral dosage form that provides a suitable drying effect, as determined by a Dosage Form Drying Capacity Assay. For example, the drying composition can include one or more drying agents having different drying capacities, and that are provided in contents by weight, that a suitable for achieving the drying effect. In one embodiment, the drying composition includes at least one drying agent having a relatively high drying capacity as determined by the Agent Drying Capacity Assay, and which may be provided in a suitable amount to impart the drying effect. However, the drying composition in one embodiment may also include at least one drying agent having a relatively low drying capacity as determined by the Agent Drying Capacity Assay, but which is provided in a relatively large content as a percent by weight of the oral dosage form to provide a suitable drying effect. Similarly, the drying composition can contain two or more different drying agents with different drying capacities and/or drying times, the combination of which drying agents imparts a suitable drying effect. Accordingly, in one embodiment, the oral dosage form has a drying capacity for the entire oral dosage form as measured by the Dosage Form Drying Capacity Assay that is within a predetermined range. The drying capacity for the entire oral dosage form can be measured at 25° C. and 1 atmosphere of pressure by the Dosage Form Drying Capacity Assay in the same and/or similar manner as discussed above for a single drying agent, with the exception that a sample of the oral dosage form is used for measurement in the place of just a single drying agent. The drying capacity of the entire dosage form in the Dosage Form Drying Capacity Assay is thus determined by crushing and/or pressing the oral dosage form having the at least one drying agent to provide particles having a size of no more than 1 mm. An empty 50 mL centrifuge tube is weighed and the initial mass is taken as a tare weight. The crushed/pressed dosage form is added and the total mass of the tube and oral dosage form is taken as the pre-hydration mass. 40 mL of PBS at 25° C. is added to the tube and the tube is sealed. The tube is vortexed for 10 seconds to fully disperse the oral dosage form into the PBS. The oral dosage form is allowed to soak in the PBS for 15 minutes. The tube is centrifuged at 2000 RPM for 15 minutes to separate the oral dosage form from the unabsorbed PBS. The resulting supernatant is decanted, and the mass of the soaked oral dosage form in the tube is taken. The mass of fluid absorbed by the oral dosage form is determined by subtracting the pre-soak mass from the post-soak mass. This value is the mass of fluid absorbed per oral dosage form, in mg fluid/oral dosage form. In one embodiment, the drying capacity of the oral dosage form having the at least one drying agent as determined by the Dosage Form Drying Capacity Assay may be at least about 1 g fluid absorbed per oral dosage form. By way of yet a further example, in one embodiment the drying capacity of the oral dosage form having the at least one drying agent as determined by the Dosage Form Drying Capacity Assay may be at least about 3 g fluid/oral dosage form. By way of yet a further example, in one embodiment the drying capacity of the oral dosage form having the at least one drying agent as determined by the Dosage Form Drying Capacity Assay may be at least about 5 g fluid/oral dosage form. By way of yet a further example, in one embodiment the drying capacity of the oral dosage form having the at least one drying agent as determined by the Dosage Form Drying Capacity Assay may be at least about 7 g fluid/oral dosage form. By way of yet a further example, in one embodiment the drying capacity of the oral dosage form having the at least one drying agent as determined by the Dosage Form Drying Capacity Assay may be at least about 9 g fluid/oral dosage form. By way of yet a further example, in one embodiment the drying capacity of the oral dosage form having the at least one drying agent as determined by the Dosage Form Drying Capacity Assay may be at least about 12 g fluid/oral dosage form. By way of yet a further example, in one embodiment the drying capacity of the oral dosage form having the at least one drying agent as determined by the Dosage Form Drying Capacity Assay may be at least about 16 g fluid/oral dosage form. By way of yet a further example, in one embodiment the drying capacity of the oral dosage form having the at least one drying agent as determined by the Dosage Form Drying Capacity Assay may be at least about 20 g fluid/oral dosage form. In general, the drying capacity of the oral dosage form as measured by the Dosage Form Drying Capacity Assay according to one embodiment is less than about 40 g fluid/oral dosage form. For example, a drying capacity of the oral dosage form having the drying agent, as measured by the Dosage Form Drying Capacity Assay, may be from about 1 g fluid/oral dosage form to about 40 g fluid/oral dosage form, such as from about 3 g fluid/oral dosage form to about 10 g fluid/oral dosage form, and even from about 5 g fluid/oral dosage form to about 10 g fluid/oral dosage form.

In one embodiment, the at least one drying agent is selected according to a fluid uptake capacity of the drying agent, as determined by an Agent Fluid Uptake Assay. In the Agent Fluid Uptake Assay, an extent of fluid that can be absorbed by a particular drying agent (fluid uptake capacity), as measured in mg of fluid taken up by a sample of the drying agent/mg of the drying agent sample. According to the Agent Fluid Uptake Assay, the fluid uptake for the drying agent of a pH 6.0 phosphate-buffered saline solution is measured at a temperature of 25° C. and 1 atmosphere of pressure. In particular, a pH 6.0 phosphate-buffered saline (PBS) media is prepared in a ratio by volume 100/10/0.675 of deionized water/10× concentrate PBS (25.6 g Na₂HPO₄.7H₂O+80 g NaCl+2 g KH₂PO₄, brought to 1 L with H₂O)/1N hydrochloric acid. 40 mL of the PBS media is weighed out, and the mass recorded. 40 mL of the PBS media is weighed out, and the mass recorded. 500 g of a drying agent is weighed out as a drying agent sample, and the mass recorded. The drying agent sample is then immersed in the PBS media for 2 hours. The PBS media containing the drying agent sample is poured onto a polyester mesh disk filter (86×86 mesh, 4 9/16″ diameter, 0.0056″ opening) for 10 seconds, and the filter is allowed to drain an additional 60 seconds. The mass of the fluid drained through the filter is taken. A Media Uptake Ratio is calculated using the following formula:

MUR=F₀−Fᵣ/P

where MUR is the Media Uptake Ratio in mg fluid uptake/mg drying agent, F₀ is the initial mass of the fluid in mg before addition of the drying agent sample, Fᵣ is the mass of the fluid drained from the drying agent sample, and P is the initial mass of the drying agent sample. The fluid uptake capacity for the drying agent as measured by the Agent Fluid Uptake Assay is thus the MUR as determined for this 2 hour immersion time period.

According to one embodiment, the fluid uptake capacity as measured in the mg PBS media absorbed per mg of drying agent in the Agent Fluid Uptake Assay is at least about 1 mg fluid/mg drying agent. By way of further example, in one embodiment the fluid uptake capacity of the drying agent as measured by the Agent Fluid Uptake Assay is at least about 3 mg fluid/mg drying agent. By way of further example, in one embodiment the fluid uptake capacity of the drying agent as measured by the Agent Fluid Uptake Assay is at least about 4 mg fluid/mg drying agent. By way of further example, in one embodiment the fluid uptake capacity of the drying agent as measured by the Agent Fluid Uptake Assay is at least about 5 mg fluid/mg drying agent. By way of further example, in one embodiment the fluid uptake capacity of the drying agent as measured by the Agent Fluid Uptake Assay is at least about 6 mg fluid/mg drying agent. By way of even further example, in one embodiment the fluid uptake capacity of the drying agent as measured by the Agent Fluid Uptake Assay is at least about 7 mg fluid/mg drying agent. By way of further example, in one embodiment the fluid uptake capacity of the drying agent as measured by the Agent Fluid Uptake Assay is at least about 10 mg fluid/mg drying agent. By way of further example, in one embodiment the fluid uptake capacity of the drying agent as measured by the Agent Fluid Uptake Assay is at least about 20 mg fluid/mg drying agent. By way of further example, in one embodiment the fluid uptake capacity of the drying agent as measured by the Agent Fluid Uptake Assay is at least about 40 mg fluid/mg drying agent. By way of further example, in one embodiment the fluid uptake capacity of the drying agent as measured by the Agent Fluid Uptake Assay is at least about 60 mg fluid/mg drying agent. By way of further example, in one embodiment the fluid uptake capacity of the drying agent as measured by the Agent Fluid Uptake Assay is at least about 80 mg fluid/mg drying agent. By way of further example, in one embodiment the fluid uptake capacity of the drying agent as measured by the Agent Fluid Uptake Assay is at least about 100 mg fluid/mg drying agent. In one embodiment, the fluid uptake capacity of the drying agent as measured by the Agent Fluid Uptake Assay will be less than about 200 mg fluid/mg drying agent, and may even be less than about 150 mg fluid/mg drying agent. In one embodiment, the fluid uptake capacity of the drying agent as measured by the Agent Fluid Uptake Assay is from about 5 mg fluid/mg drying agent to about 200 mg fluid/mg drying agent, such as from about 10 mg fluid/mg drying agent to about 150 mg fluid/mg drying agent, and even from about 20 mg fluid/mg drying agent to about 100 mg fluid/mg drying agent.

In one embodiment, the at least one drying agent is selected according to a fluid uptake time of the agent, as determined according to an Agent Fluid Uptake Time Assay described herein. In the Agent Fluid Uptake Time Assay, the Agent Fluid Uptake Assay described above is performed to determine the media uptake ratio (MUR) at different time points. For example, the drying agent sample may be allowed to soak in the PBS media for a number of different time periods time periods ranging from 10 seconds to 2 hours, such as time periods of 1 minute, 5 minutes, 10 minutes, 20 minutes, 30 minutes, 40 minutes, 1 hour and 2 hours. The MURs from all time points are compared, and the time for fluid uptake according to the Agent Fluid Uptake Time Assay is determined by selecting the earliest time point that achieves 90% of the MUR that is established as the extent of the fluid that can be absorbed by the drying agent (the fluid uptake capacity) by the Agent Fluid Uptake Assay. If none of the evaluated time points achieve 90% of the fluid uptake capacity, i.e. if the evaluated time points exhibit MURs that are either below or above 90% of the fluid uptake capacity, then new time points are selected that are either above or below the evaluated time points, and the process of determining the MUR for the newly selected time points is repeated, until a time point is identified where the drying agent exhibits 90% of its fluid uptake capacity.

According to one embodiment, the fluid uptake time as measured in seconds in the Agent Fluid Uptake Time Assay is no more than about 5 seconds. By way of further example, in one embodiment the fluid uptake time of the drying agent as measured by the Agent Fluid Uptake Time Assay is no more than about 30 seconds. By way of even further example, in one embodiment the fluid uptake time of the drying agent as measured by the Agent Fluid Uptake Time Assay is no more than about 60 seconds. By way of even further example, in one embodiment the fluid uptake time of the drying agent as measured by the Agent Fluid Uptake Time Assay is no more than about 300 seconds (5 minutes). By way of even further example, in one embodiment the fluid uptake time of the drying agent as measured by the Agent Fluid Uptake Time Assay is no more than about 600 seconds (10 minutes). By way of even further example, in one embodiment the fluid uptake time of the drying agent as measured by the Agent Fluid Uptake Time Assay is no more than about 900 seconds (15 minutes). In general, the fluid uptake time of the drying agent as measured by the Agent Fluid Uptake Time Assay will be less that about 1800 seconds (30 minutes). By way of even further example, in one embodiment the fluid uptake time of the drying agent as measured by the Agent Fluid Uptake Time Assay is no more than about 1 hour. By way of even further example, in one embodiment the fluid uptake time of the drying agent as measured by the Agent Fluid Uptake Time Assay is no more than about 1½ hours. By way of even further example, in one embodiment the fluid uptake time of the drying agent as measured by the Agent Fluid Uptake Time Assay is no more than about 2 hours. In one embodiment, the fluid uptake time of the drying agent as measured by the Agent Fluid Uptake Time Assay will be no more than about 3 hours. In one embodiment, the fluid uptake time will be at least 1 second. In one embodiment, the fluid uptake time of the drying agent as measured by the Agent Fluid Uptake Time Assay is from about 5 seconds to about 3 hours, such as from about 10 minutes seconds to about 2 hours, and even from about 20 minutes seconds to about 1½ hours.

In one embodiment, the composition and content of drying composition of the oral dosage form is selected to provide a fluid uptake capacity for the entire oral dosage form that provides a suitable drying effect, as determined by a Dosage Form Fluid Uptake Assay. For example, the drying composition can include one or more drying agents having different fluid uptake capacities, and that are provided in contents by weight, that are suitable for achieving the drying effect. In one embodiment, the drying composition includes at least one drying agent having a relatively high fluid uptake capacity as determined by the Agent Fluid Uptake Assay, and which may be provided in a suitable amount to impart the drying effect. However, the drying composition in one embodiment may also include at least one drying agent having a relatively low fluid uptake capacity as determined by the Agent Fluid Uptake Assay, but which is provided in a relatively large content as a percent by weight of the oral dosage form to provide a suitable drying effect. Similarly, the drying composition can contain two or more different drying agents with different fluid uptake capacities and/or drying times, the combination of which drying agents imparts a suitable drying effect. Accordingly, in one embodiment, the oral dosage form has a fluid uptake capacity for the entire oral dosage form as measured by the Dosage Form Fluid Uptake Assay that is within a predetermined range.

In the Dosage Form Fluid Uptake Assay, an extent of fluid that can be absorbed by a particular dosage from (fluid uptake capacity) is determined, as measured in mg of fluid taken up by a dosage form. According to the Dosage Form Fluid Uptake Assay, the fluid uptake for the dosage form of a pH 6.0 phosphate-buffered saline solution is measured at a temperature of 25° C. and 1 atmosphere of pressure. In particular, a pH 6.0 phosphate-buffered saline (PBS) media is prepared in a ratio by volume 100/10/0.675 of deionized water/10× concentrate PBS (25.6 g Na₂HPO₄.7H₂O+80 g NaCl+2 g KH₂PO₄, brought to 1 L with H₂O)/1N hydrochloric acid. 150 mL of the PBS media is weighed out, and the mass recorded. The oral dosage form is crushed and/or pressed to provide particles having a size of no more than 1 mm. The dosage form particles are then immersed in the PBS media for 2 hours. The PBS media containing the dosage form particles is poured onto a polyester mesh disk filter (86×86 mesh, 4 9/16″ diameter, 0.0056″ opening) for 10 seconds, and the filter is allowed to drain an additional 60 seconds. The mass of the fluid drained through the filter is taken. A total dosage form Media Uptake MU_{D} is calculated using the following formula:

MU_{D}=F₀−Fᵣ

where MU_{D} is the total Media Uptake for the dosage form in mg fluid uptake per dosage form, F₀ is the initial mass of the fluid in mg before addition of the dosage form particles, and Fᵣ is the mass of the fluid drained from the dosage form particles. The fluid uptake capacity for the dosage form as measured by the Dosage Form Fluid Uptake Assay is thus the MU_{D} as determined for this 2 hour immersion time period.

In one embodiment, the fluid uptake capacity of the oral dosage form having the at least one drying agent as determined by the Dosage Form Fluid Uptake Assay may be at least about 1 g fluid absorbed per oral dosage form. By way of yet a further example, in one embodiment the fluid uptake capacity of the oral dosage form having the at least one drying agent as determined by the Dosage Form Fluid Uptake Assay may be at least about 3 g fluid/oral dosage form. By way of yet a further example, in one embodiment the fluid uptake capacity of the oral dosage form having the at least one drying agent as determined by the Dosage Form Fluid Uptake Assay may be at least about 5 g fluid/oral dosage form. By way of yet a further example, in one embodiment the fluid uptake capacity of the oral dosage form having the at least one drying agent as determined by the Dosage Form Fluid Uptake Assay may be at least about 7 g fluid/oral dosage form. By way of yet a further example, in one embodiment the fluid uptake capacity of the oral dosage form having the at least one drying agent as determined by the Dosage Form Fluid Uptake Assay may be at least about 9 g fluid/oral dosage form. By way of yet a further example, in one embodiment the fluid uptake capacity of the oral dosage form having the at least one drying agent as determined by the Dosage Form Fluid Uptake Assay may be at least about 12 g fluid/oral dosage form. By way of yet a further example, in one embodiment the fluid uptake capacity of the oral dosage form having the at least one drying agent as determined by the Dosage Form Fluid Uptake Assay may be at least about 16 g fluid/oral dosage form. By way of yet a further example, in one embodiment the fluid uptake capacity of the oral dosage form having the at least one drying agent as determined by the Dosage Form Fluid Uptake Assay may be at least about 20 g fluid/oral dosage form. By way of yet a further example, in one embodiment the fluid uptake capacity of the oral dosage form having the at least one drying agent as determined by the Dosage Form Fluid Uptake Assay may be at least about 40 g fluid/oral dosage form. By way of yet a further example, in one embodiment the fluid uptake capacity of the oral dosage form having the at least one drying agent as determined by the Dosage Form Fluid Uptake Assay may be at least about 60 g fluid/oral dosage form. By way of yet a further example, in one embodiment the fluid uptake capacity of the oral dosage form having the at least one drying agent as determined by the Dosage Form Fluid Uptake Assay may be at least about 80 g fluid/oral dosage form. By way of yet a further example, in one embodiment the fluid uptake capacity of the oral dosage form having the at least one drying agent as determined by the Dosage Form Fluid Uptake Assay may be at least about 100 g fluid/oral dosage form. In general, the fluid uptake capacity of the oral dosage form as measured by the Dosage Form Fluid Uptake Assay according to one embodiment is less than about 200 g fluid/oral dosage form. For example, a fluid uptake capacity of the oral dosage form having the drying agent, as measured by the Dosage Form Fluid Uptake Assay, may be from about 1 g fluid/oral dosage form to about 100 g fluid/oral dosage form, such as from about 3 g fluid/oral dosage form to about 80 g fluid/oral dosage form, and even from about 5 g fluid/oral dosage form to about 60 g fluid/oral dosage form.

In one embodiment, the at least one drying agent and/or drying agents are selected according to a fluid uptake time of the dosage form having the at least one drying agent, at a certain pH, as determined according to an Dosage Form Fluid Uptake Time Assay at pH described herein. In the Dosage Form Fluid Uptake Time Assay at pH, a dosage form having a pH-dependent coating, such as an enteric coating, is immersed in a phosphate buffer solution (PBS) at 25° C. and 1 atmosphere of pressure, and the pH of the solution is adjusted to a predetermined pH at which the pH-dependent coating dissolves and/or becomes permeable (if not already at that pH at the point of immersion), such as a pH of at least 7.4. A relative mass of the fluid absorbed by the dosage form over a range of set time periods is measured at 25° C. and 1 atmosphere of pressure at the predetermined pH. In particular, the relative mass of a phosphate buffer solution (PBS) at 25° C. that is absorbed by the entire dosage form at the predetermined pH over multiple set time periods is determined, to evaluate the drying time of the dosage form. For example, the time periods at which the fluid uptake is evaluated may be time periods ranging from 10 seconds to 2 hours, such as time periods of 1 minute, 5 minutes, 10 minutes, 20 minutes, 30 minutes, 40 minutes, 1 hour and 2 hours. According to the Dosage Form Fluid Uptake Time Assay at pH, a pH 6.0 phosphate-buffered saline (PBS) media is prepared in a ratio by volume 100/10/0.675 of deionized water/10× concentrate PBS (25.6 g Na₂HPO₄.7H₂O+80 g NaCl+2 g KH₂PO₄, brought to 1 L with H₂O)/1N hydrochloric acid. 150 mL of the PBS media is weighed out, and the mass recorded. The dosage form is then immersed in the PBS media, and the pH is adjusted to pH 7.4 to at least partially dissolve and/or render permeable the enteric coating. The dosage form is allowed to soak in the PBS media for the predetermined time period, after which the PBS media containing the dosage form is poured onto a polyester mesh disk filter (86×86 mesh, 4 9/16″ diameter, 0.0056″ opening) for 10 seconds, and the filter is allowed to drain an additional 60 seconds. The mass of the fluid drained through the filter is taken. The total media uptake MU_{D} for the entire dosage form is calculated, as in the Dosage Form Fluid Uptake Assay above, to determine an extent of fluid uptake for the entire dosage form at the time point, as measured in mg fluid absorbed per dosage form. In particular, the MU_{D} is determined according to the following formula:

MU_{D}=F₀−Fᵣ

where MU_{D} is the total Media Uptake Ratio for the dosage form in mg fluid uptake per dosage form, F₀ is the initial mass of the fluid in mg before addition of the dosage form, and Fᵣ is the mass of the fluid drained from the dosage form. This procedure is repeated, changing the time in which the dosage form soaks in PBS. The mg fluid absorbed/dosage form from all time points will be compared and the fluid uptake time is determined by selecting the earliest time point that achieves 90% of the dosage form fluid uptake capacity as determined by the Dosage Form Fluid Uptake Assay for the same dosage form. If none of the evaluated time points achieve 90% of the fluid uptake capacity for the dosage form, i.e. if the evaluated time points exhibit MU_{D}s that are either below or above 90% of the fluid uptake capacity for the dosage form, then new time points are selected that are either above or below the evaluated time points, and the process of determining the MU_{D} for the newly selected time points is repeated, until a time point is identified where the dosage form exhibits 90% of its fluid uptake capacity.

According to one embodiment, the fluid uptake time for the dosage form as measured in seconds in the Dosage Form Fluid Uptake Time Assay at pH is no more than about 5 seconds. By way of further example, in one embodiment the fluid uptake time of the dosage form as measured by the Dosage Form Fluid Uptake Time Assay at pH is no more than about 30 seconds. By way of even further example, in one embodiment the fluid uptake time of the dosage form as measured by the Dosage Form Fluid Uptake Time Assay at pH is no more than about 60 seconds. By way of even further example, in one embodiment the fluid uptake time of the dosage form as measured by the Dosage Form Fluid Uptake Time Assay at pH is no more than about 300 seconds (5 minutes). By way of even further example, in one embodiment the fluid uptake time of the dosage form as measured by the Dosage Form Fluid Uptake Time Assay at pH is no more than about 600 seconds (10 minutes). By way of even further example, in one embodiment the fluid uptake time of the dosage form as measured by the Dosage Form Fluid Uptake Time Assay at pH is no more than about 900 seconds (15 minutes). In general, the fluid uptake time of the dosage form as measured by the Dosage Form Fluid Uptake Time Assay at pH will be less that about 1800 seconds (30 minutes). By way of even further example, in one embodiment the fluid uptake time of the dosage form as measured by the Dosage Form Fluid Uptake Time Assay at pH is no more than about 1 hour. By way of even further example, in one embodiment the fluid uptake time of the dosage form as measured by the Dosage Form Fluid Uptake Time Assay at pH is no more than about 1½ hours. By way of even further example, in one embodiment the fluid uptake time of the dosage form as measured by the Dosage Form Fluid Uptake Time Assay at pH is no more than about 2 hours. In one embodiment, the fluid uptake time of the dosage form as measured by the Dosage Form Fluid Uptake Time Assay at pH will be no more than about 3 hours. In one embodiment, the fluid uptake time of the dosage form as measured by the Dosage Form Fluid Uptake Time Assay at pH will be at least 1 second. In one embodiment, the fluid uptake time of the of the dosage form as measured by the Dosage Form Fluid Uptake Time Assay at pH is from about 5 seconds to about 3 hours, such as from about 10 minutes seconds to about 2 hours, and even from about 20 minutes seconds to about 1½ hours.

In one embodiment, the at least one drying agent and/or drying agents are selected according to a fluid uptake time of the dosage form having the at least one drying agent, at a certain phase of the fluid uptake, as determined according to an Dosage Form Fluid Uptake Time Assay at Uptake Phase, as described herein. In the Dosage Form Fluid Uptake Time Assay for Uptake Phase, a time required for a phase of fluid uptake by the dosage form to occur is determined, the fluid uptake phase occurring during a predetermined time in which the dosage form goes a first percentage of its fluid uptake capacity to a second percentage of its fluid uptake capacity, as measured by the Dosage Form Fluid Uptake Assay. For example, the fluid uptake phase may correspond to a time period in which the percentage of the fluid uptake of the dosage form achieved by absorption of the fluid increases by at least 50%, such as a time period required for the dosage form to absorb from 0% of its total possible fluid uptake capacity to 50% of its fluid uptake capacity, and/or a time period required for the dosage form to absorb from 5% to 55% of its fluid uptake capacity, and/or a time period required for the dosage form to absorb from 10% to 60% of its fluid uptake capacity, and/or a time period required for the dosage form to absorb from 25% to 75% of its fluid uptake capacity, and/or a time period required for the dosage form to absorb from 50% to 100% of its fluid uptake capacity.

In the Dosage Form Fluid Uptake Time for Drying Phase, a dosage form is immersed in phosphate buffer solution (PBS) at 25° C. and 1 atmosphere of pressure. A relative mass of the fluid absorbed by the dosage form over a range of set time periods is measured at 25° C. and 1 atmosphere of pressure, to determine the time it takes for the dosage form to absorb an amount of fluid in the predetermined drying phase. According to the Dosage Form Fluid Uptake Time Assay for Uptake Phase, a pH 6.0 phosphate-buffered saline (PBS) media is prepared in a ratio by volume 100/10/0.675 of deionized water/10× concentrate PBS (25.6 g Na₂HPO₄.7H₂O+80 g NaCl+2 g KH₂PO₄, brought to 1 L with H₂O)/1N hydrochloric acid. 150 mL of the PBS media is weighed out, and the mass recorded. The dosage form is then immersed in the PBS media, and allowed to soak at least until the dosage form begins to at least partially dissolve in the media. For example, a time until the dosage form begins to dissolve in the fluid media may be in the range of from 10 seconds to up 8 or 24 hours or longer, depending on the composition of the dosage form. Once the dosage form begins to at least partially dissolve in the fluid media, immersion of the dosage form in the fluid media is continued for a predetermined period of time. For example, the predetermined period of time after the dosage form has begun to at least partially dissolve may be a time period in the range of from 10 seconds to 2 hours, such as time periods of 1 minute, 5 minutes, 10 minutes, 20 minutes, 30 minutes, 40 minutes, 1 hour and 2 hours. After the predetermined period of time has passed, the PBS media containing the dosage form is poured onto a polyester mesh disk filter (86×86 mesh, 4 9/16″ diameter, 0.0056″ opening) for 10 seconds, and the filter is allowed to drain an additional 60 seconds. The mass of the fluid drained through the filter is taken. The total media uptake MU_{D} for the entire dosage form is calculated, as in the Dosage Form Fluid Uptake Assay above, to determine an extent of fluid uptake for the entire dosage form at the time point, as measured in mg fluid absorbed per dosage form. In particular, the MU_{D} is determined according to the following formula:

MU_{D}=F₀−Fᵣ

where MU_{D} is the total Media Uptake in mg fluid uptake per dosage form, F₀ is the initial mass of the fluid in mg before addition of the dosage form, and Fᵣ is the mass of the fluid drained from the dosage form. This procedure is repeated, changing the time in which the dosage form soaks in the PBS fluid. The mg fluid absorbed/dosage form from all time points are compared to determine one or more fluid uptake phases where at least a 50% increase in the amount of fluid absorbed by the dosage form is achieved (e.g., a phase where a change in the fluid uptake extent is from 25% to 75%). The fluid uptake time for the one or more fluid uptake phases, such as a phase that achieves at least a 50% increase in the amount of fluid absorbed (e.g., from a fluid uptake extent of 25% to a fluid uptake extent of 75%), is then determined by taking a difference between the time points representing the end points of the phase. For example, the fluid uptake time may be the difference between the time point at which 25% of the fluid uptake capacity is achieved, and the time point at which 75% of the fluid uptake capacity is achieved, the fluid uptake capacity being determined by the Dosage Form Fluid Uptake Assay for the same dosage form. Accordingly, a time required for a fluid uptake phase in which the dosage form increases fluid uptake by at least 50% can be determined.

In one embodiment, the dosage form has a fluid uptake time as measured by the Dosage Form Fluid Uptake Time Assay for Uptake Phase for at least one fluid uptake phase, the phase corresponding to at least a 50% increase in the amount of fluid absorbed, that is no more than about 5 seconds. By way of further example, in one embodiment the dosage form has a fluid uptake time as measured by the Dosage Form Fluid Uptake Time Assay for Uptake Phase for at least one fluid uptake phase, the phase corresponding to at least a 50% increase in the amount of fluid absorbed, that is no more than about 30 seconds. By way of even further example, in one embodiment in one embodiment the dosage form has a fluid uptake time as measured by the Dosage Form Fluid Uptake Time Assay for Uptake Phase for at least one fluid uptake phase, the phase corresponding to at least a 50% increase in the amount of fluid absorbed, that is no more than about 60 seconds. By way of even further example, in one embodiment the dosage form has a fluid uptake time as measured by the Dosage Form Fluid Uptake Time Assay for Uptake Phase for at least one fluid uptake phase, the phase corresponding to at least a 50% increase in the amount of fluid absorbed, that is no more than about 300 seconds (5 minutes). By way of even further example, in one embodiment the dosage form has a fluid uptake time as measured by the Dosage Form Fluid Uptake Time Assay for Uptake Phase for at least one fluid uptake phase, the phase corresponding to at least a 50% increase in the amount of fluid absorbed, that is no more than about 600 seconds (10 minutes). By way of even further example, in one embodiment the dosage form has a fluid uptake time as measured by the Dosage Form Fluid Uptake Time Assay for Uptake Phase for at least one fluid uptake phase, the phase corresponding to at least a 50% increase in the amount of fluid absorbed, that is no more than about 900 seconds (15 minutes). In one embodiment, the dosage form has a fluid uptake time as measured by the Dosage Form Fluid Uptake Time Assay for Uptake Phase for at least one fluid uptake phase, the phase corresponding to at least a 50% increase in the amount of fluid absorbed, that will be no more than about 1800 seconds (30 minutes). By way of even further example, in one embodiment the dosage form has a fluid uptake time as measured by the Dosage Form Fluid Uptake Time Assay for Uptake Phase for at least one fluid uptake phase, the phase corresponding to at least a 50% increase in the amount of fluid absorbed, that is no more than about 1 hour. By way of even further example, in one embodiment the dosage form has a fluid uptake time as measured by the Dosage Form Fluid Uptake Time Assay for Uptake Phase for at least one fluid uptake phase, the phase corresponding to at least a 50% increase in the amount of fluid absorbed, that is no more than about 1½ hours. By way of even further example, in one embodiment the dosage form has a fluid uptake time as measured by the Dosage Form Fluid Uptake Time Assay for Uptake Phase for at least one fluid uptake phase, the phase corresponding to at least a 50% increase in the amount of fluid absorbed, that is no more than about 2 hours. In one embodiment, the dosage form has a fluid uptake time as measured by the Dosage Form Fluid Uptake Time Assay for Uptake Phase for at least one fluid uptake phase, the phase corresponding to at least a 50% increase in the amount of fluid absorbed, that will be no more than about 3 hours. In one embodiment, in one embodiment the dosage form has a fluid uptake time as measured by the Dosage Form Fluid Uptake Time Assay for Uptake Phase for at least one fluid uptake phase, the phase corresponding to at least a 50% increase in the amount of fluid absorbed, that will be at least 1 second. In one embodiment, the dosage form has a fluid uptake time as measured by the Dosage Form Fluid Uptake Time Assay for Uptake Phase for at least one fluid uptake phase, the phase corresponding to at least a 50% increase in the amount of fluid absorbed, that is from about 5 seconds to about 3 hours, such as from about 10 minutes seconds to about 2 hours, and even from about 20 minutes seconds to about 1½ hours.

In one embodiment, the at least one drying agent and/or drying agents are selected according to a fluid uptake time of the dosage form having the at least one drying agent, as determined according to an Dosage Form Fluid Uptake Time Assay at Breakthrough, as described herein. In the Dosage Form Fluid Uptake Time Assay at Breakthrough, a time in which a predetermined extent of fluid uptake by an entire dosage form is provided (fluid uptake capacity) is determined, when the dosage form is crushed and/or pressed into particles, so that contents of the dosage form that would otherwise be covered by a protective coating can be exposed to fluid. In the Dosage Form Fluid Uptake Time Assay at Breakthrough, the dosage form is crushed and/or pressed to provide particles having a size of no more than 1 mm. The dosage form particles are immersed in a phosphate buffer solution (PBS) at 25° C. and 1 atmosphere of pressure. A relative mass of the fluid absorbed by the dosage form particles over a range of set time periods is measured at 25° C. and 1 atmosphere of pressure, to determine the time it takes for the dosage form particles to absorb an amount of fluid. For example, the time periods at which the fluid uptake is evaluated may be time periods ranging from 10 seconds to 2 hours, such as time periods of 1 minute, 5 minutes, 10 minutes, 20 minutes, 30 minutes, 40 minutes, 1 hour and 2 hours. According to the Dosage Form Fluid Uptake Time Assay at Breakthrough, a pH 6.0 phosphate-buffered saline (PBS) media is prepared in a ratio by volume 100/10/0.675 of deionized water/10× concentrate PBS (25.6 g Na₂HPO₄.7H₂O+80 g NaCl+2 g KH₂PO₄, brought to 1 L with H₂O)/1N hydrochloric acid. 150 mL of the PBS media is weighed out, and the mass recorded. The dosage form particles are then immersed in the PBS media. The dosage form particles allowed to soak in the PBS media for the predetermined time period, after which the PBS media containing the dosage form particles is poured onto a polyester mesh disk filter (86×86 mesh, 4 9/16″ diameter, 0.0056″ opening) for 10 seconds, and the filter is allowed to drain an additional 60 seconds. The mass of the fluid drained through the filter is taken. The total media uptake MU_{D} for the entire dosage form is calculated, as in the Dosage Form Fluid Uptake Assay above, to determine an extent of fluid uptake for the dosage form particles at the time point, as measured in mg fluid absorbed per dosage form. In particular, the MU_{D} is determined according to the following formula:

MU_{D}=F₀−Fᵣ

where MU_{D} is the total Media Uptake for the dosage form in mg fluid uptake per dosage form, F₀ is the initial mass of the fluid in mg before addition of the dosage form particles, and Fᵣ is the mass of the fluid drained from the divided dosage form. This procedure is repeated, changing the time in which the dosage form particles soak in PBS. The mg fluid absorbed/dosage form from all time points will be compared and the fluid uptake time is determined by selecting the earliest time point that achieves 90% of the dosage form fluid uptake capacity as determined by the Dosage Form Fluid Uptake Assay for the same dosage form. If none of the evaluated time points achieve 90% of the fluid uptake capacity for the dosage form, i.e. if the evaluated time points exhibit MU_{D}s that are either below or above 90% of the fluid uptake capacity for the dosage form, then new time points are selected that are either above or below the evaluated time points, and the process of determining the MU_{D} for the newly selected time points is repeated, until a time point is identified where the dosage form exhibits 90% of its fluid uptake capacity.

According to one embodiment, the fluid uptake time as measured in seconds according to the Dosage Form Fluid Uptake Time Assay at Breakthrough is no more than about 5 seconds. By way of further example, in one embodiment the fluid uptake time of the dosage form as measured by the Dosage Form Fluid Uptake Time Assay at Breakthrough is no more than about 30 seconds. By way of even further example, in one embodiment the fluid uptake time of the dosage form as measured by the Dosage Form Fluid Uptake Time Assay at Breakthrough is no more than about 60 seconds. By way of even further example, in one embodiment the fluid uptake time of the dosage form as measured by the Dosage Form Fluid Uptake Time Assay at Breakthrough is no more than about 300 seconds (5 minutes). By way of even further example, in one embodiment the fluid uptake time of the dosage form as measured by the Dosage Form Fluid Uptake Time Assay at Breakthrough is no more than about 600 seconds (10 minutes). By way of even further example, in one embodiment the fluid uptake time of the dosage form as measured by the Dosage Form Fluid Uptake Time Assay at Breakthrough is no more than about 900 seconds (15 minutes). In general, the fluid uptake time of the dosage form as measured by the Dosage Form Fluid Uptake Time Assay at Breakthrough will be less that about 1800 seconds (30 minutes). By way of even further example, in one embodiment the fluid uptake time of the dosage form as measured by the Dosage Form Fluid Uptake Time Assay at Breakthrough is no more than about 1 hour. By way of even further example, in one embodiment the fluid uptake time of the dosage form as measured by the Dosage Form Fluid Uptake Time Assay at Breakthrough is no more than about 1½ hours. By way of even further example, in one embodiment the fluid uptake time of the dosage form as measured by the Dosage Form Fluid Uptake Time Assay at Breakthrough is no more than about 2 hours. In one embodiment, the fluid uptake time of the dosage form as measured by the Dosage Form Fluid Uptake Time Assay at Breakthrough will be no more than about 3 hours. In one embodiment, the fluid uptake time of the dosage form as measured by the Dosage Form Fluid Uptake Time Assay at Breakthrough will be at least 1 second. In one embodiment, the fluid uptake time of the of the dosage form as measured by the Dosage Form Fluid Uptake Time Assay at Breakthrough is from about 5 seconds to about 3 hours, such as from about 10 minutes seconds to about 2 hours, and even from about 20 minutes seconds to about 1½ hours.

In addition and/or alternatively, in one embodiment, a drying capacity and/or fluid uptake capacity for the portion of the oral dosage form comprising the drying composition having the one or more drying agents therein, may be selected to be within a predetermined range as measured by the Agent Drying Capacity Assay and/or Agent Fluid Uptake Assay. The drying capacity and/or fluid uptake capacity for the drying composition can be measured by the Agent Drying Capacity Assay and/or Agent Fluid Uptake Capacity in the same manner as discussed above for a single drying agent, with the exception that a sample of the drying composition (e.g., possibly containing binder, gelling agent, and other ingredients, along with one or more drying agents) is used for measurement in the place of just a single drying agent, and the amount of fluid absorbed for the entire drying composition used in the oral dosage form is determined. In one embodiment, the drying capacity and/or fluid uptake capacity of the drying composition having the at least one drying agent as determined by the Agent Drying Capacity Assay and/or Agent Fluid Uptake Assay may be at least about 1 mg fluid/mg drying composition. By way of further example, in one embodiment the drying capacity and/or fluid uptake capacity of the drying composition having the at least one drying agent as determined by the Agent Drying Capacity Assay and/or Agent Fluid Uptake Assay may be at least about 4 mg fluid/mg drying composition. By way of yet a further example, in one embodiment the drying capacity and/or fluid uptake capacity of the drying composition having the at least one drying agent as determined by the Agent Drying Capacity Assay and/or Agent Fluid Uptake Assay may be at least about 7 mg fluid/mg drying composition. By way of yet a further example, in one embodiment the drying capacity and/or fluid uptake capacity of the drying composition having the at least one drying agent as determined by the Agent Drying Capacity Assay and/or Agent Fluid Uptake Assay may be at least about 10 mg fluid/mg drying composition. By way of yet a further example, in one embodiment the drying capacity and/or fluid uptake capacity of the drying composition having the at least one drying agent as determined by the Agent Drying Capacity Assay and/or Agent Fluid Uptake Assay may be at least about 20 mg fluid/mg drying composition. By way of yet a further example, in one embodiment the drying capacity and/or fluid uptake capacity of the drying composition having the at least one drying agent as determined by the Agent Drying Capacity Assay and/or Agent Fluid Uptake Assay may be at least about 40 mg fluid/mg drying composition. By way of yet a further example, in one embodiment the drying capacity and/or fluid uptake capacity of the drying composition having the at least one drying agent as determined by the Agent Drying Capacity Assay and/or Agent Fluid Uptake Assay may be at least about 60 mg fluid/mg drying composition. By way of yet a further example, in one embodiment the drying capacity and/or fluid uptake capacity of the drying composition having the at least one drying agent as determined by the Agent Drying Capacity Assay and/or Agent Fluid Uptake Assay may be at least about 80 mg fluid/mg drying composition. By way of yet a further example, in one embodiment the drying capacity and/or fluid uptake capacity of the drying composition having the at least one drying agent as determined by the Agent Drying Capacity Assay and/or Agent Fluid Uptake Assay may be at least about 100 mg fluid/mg drying composition. In general, the drying capacity and/or fluid uptake capacity of the drying composition as measured by the Agent Drying Capacity Assay and/or Agent Fluid Uptake Assay according to one embodiment is less than about 100 mg fluid/mg drying composition. For example, a drying capacity and/or fluid uptake capacity of the drying composition portion of the oral dosage form having the drying agent, as measured by the Agent Drying Capacity Assay and/or Agent Fluid Uptake Assay, may be from about 1 mg fluid/mg drying composition to about 100 mg fluid/drying composition, and even from about 1 mg fluid/mg drying composition to about 40 mg fluid/mg drying composition, such as from about 5 mg fluid/mg drying composition to about 80 mg fluid/mg drying composition, such as from about 5 mg fluid/mg drying composition to about 20 mg fluid/mg drying composition, and even from about 10 mg fluid/mg drying composition to about 60 mg fluid/mg drying composition, such as from about 10 mg fluid/mg drying composition to about 20 mg fluid/mg drying composition.

In one embodiment, the at least one drying agent comprises an agent with relatively low solubility in an aqueous solution. Without being limited to any one theory, it is believed that a relatively low solubility drying agent may be better capable of retaining its drying capacity upon release from the oral dosage form at the intestinal site, as compared to agents that otherwise readily dissolve in and/or form a gel in aqueous solution. For example, according to one embodiment, the drying agent may be sufficiently insoluble such that it does not excessively gel and/or increase the viscosity of an aqueous fluid in the immediate vicinity of the drying agent. According to one embodiment, an increase in viscosity of an aqueous solution due to the drying agent is sufficiently low that a viscosity of a liquid part of a solution of water at standard temperature and pressure containing 5 mg of the drying agent/mL water is less than 1 cP. By way of further example, in one embodiment a viscosity of a liquid part of a solution of water at standard temperature and pressure containing 5 mg of the drying agent/mL water is less than 10 cP. By way of further example, in one embodiment a viscosity of a liquid part of a solution of water at standard temperature and pressure containing 5 mg of the drying agent/mL water is less than 50 cP. By way of further example, in one embodiment a viscosity of a liquid part of a solution of water at standard temperature and pressure containing 5 mg of the drying agent/mL water is less than 100 cP. By way of further example, in one embodiment a viscosity of a liquid part of a solution of water at standard temperature and pressure containing 5 mg of the drying agent/mL water is less than 500 cP. By way of further example, in one embodiment a viscosity of a liquid part of a solution of water at standard temperature and pressure containing 5 mg of the drying agent/mL water is less than 1000 cP. By way of further example, in one embodiment a viscosity of a liquid part of a solution of water at standard temperature and pressure containing 5 mg of the drying agent/mL water is less than 10,000 cP.

In one embodiment, the drying composition and/or the at least one drying agent is provided in the form of a particle. The particles provided in the dosage form retain boundaries and/or surfaces therebetween, even if pressed into a macroscopic tablet. For example, the drying composition and/or at least one drying agent may be provided in the form of a population of particles having a weight average dry particle size, P_{avg}, of at least 0.02 microns. For example, in one such embodiment, the population has a weight average dry particle size, P_{avg}, of at least 0.5 microns. By way of further example, in one such embodiment the population has a weight average dry particle size, P_{avg}, of at least 5 microns. By way of further example, in one such embodiment the population has a weight average dry particle size, P_{avg}, of at least 20 microns. By way of further example, in one such embodiment the population has a weight average dry particle size, P_{avg}, of at least 100 microns. By way of further example, in one such embodiment the population has a weight average dry particle size, P_{avg}, of at least 500 microns. By way of further example, in one such embodiment the population has a weight average dry particle size, P_{avg}, of at least 1000 microns. In general, however, any such population will have a weight average dry particle size P_{avg} of less than 2000 microns. By way of further example, in one such embodiment the population has a weight average dry particle size, P_{avg}, of less than 1000 microns. By way of further example, in one such embodiment the population has a weight average dry particle size, P_{avg}, of less than 400 microns. By way of further example, in one such embodiment the population has a weight average dry particle size, P_{avg}, of less than 200 microns. For example, the population in one embodiment has a weight average dry particle size, P_{avg}, of from about 0.01 microns to about 1000 microns, such as from about 1 micron to about 500 microns. In one embodiment, the population has a weight average dry particle size, P_{avg} of from about 10 microns to about 1000 microns, such as about 50 microns to about 500 microns, including from about 100 microns to about 400 microns, and even from about 20 microns to about 200 microns. The particle size can be measured, in one embodiment, according to a laser diffraction method, such as that described in the ISO 13320:2009 standard. In one embodiment, particles having a smaller particle size may be capable of absorbing fluid at a faster rate than relatively larger particles. For example, according to one embodiment, particles having a weight average dry particle size, P_{avg}, of less than 500 microns, and even less than 400 microns, may have a faster drying time and/or fluid uptake time as measured by any of the Assays above as compared to particles having larger weight average dry particle sizes.

Without being limited by any particular theory, it is believed that by providing the drying agent in a particle form (such as in the form of compressed particles in a tablet or mini-tablet form, or otherwise in a loose particle form) the drying agent may be capable of effectively imparting the drying effect, without requiring an excessively large or bulky dosage form. This may be in contrast to drying agents provided in “bulk” or other non-particulate forms, which forms can be difficult to administer and may not provide desired effects in all cases. According to yet another aspect, the drying agent in particle form may be capable of relatively quickly dispersing at the targeted intestinal site, to not only provide the desired drying effect, but also such that the drying agent particles can be processed and removed from the gastrointestinal tract at a suitable rate (e.g., without excessive retention time in the GI tract). Yet a further advantage of providing the drying agent in particle form, according to one embodiment, may be in the relative ease of manufacture of dosage forms containing the drying agent particles, compared to other forms. For example, the synthesis and manufacture of dosage forms containing “bulk” forms (non-particulate forms) of certain drying agents may be sufficiently difficult as to make them commercially infeasible on a production scale. They may also be prohibitively large as a result of the bulk form that they cannot be readily swallowed, thereby reducing patient compliance, as described for example in WO 2009/125432 to Hans E. Junginger et al., published on Mar. 25, 2010, which is hereby incorporated by reference herein in its entirety.

In one embodiment, suitable drying agents that may be provided as a part of the drying composition for the oral dosage form can comprise at least one of disintegrants, super-disintegrants, dessicants, super-absorbent polymers, swellable polymers, super porous hydrogels and the like. For example, in one embodiment the at least one drying agent comprises one or more of modified cellulose/crosslinked cellulose and derivatives thereof, such as croscarmellose sodium, including Ac—Si-Sol SD-711 NF, carboxymethyl cellulose calcium, carboxymethyl cellulose sodium, hydroxypropyl cellulose, methyl cellulose, povidone, crosslinked polyvinylpyrrolidone (e.g., crospovidone), starch and/or modified starch, crosslinked starch, crosslinked alginic acid, sodium polyacrylate, cross-linked sodium polyacrylate, sodium starch glycolate, soy polysaccharide, gellan gum, xanthan gum, silicon dioxide, magnesium aluminum silicate (e.g., Neusilin), calcium silicate, and ion exchange resins. Specifically, super-distintegrants that may be suitable for the composition can include modified starches such as sodium carboxymethyl starches, including sodium starch glycolate, cross-linked polyvinlypyrrolidone such as crospovidone (e.g., Kollidon and Polyplasdone), and modified celluloses such as croscarmellose sodium. In one embodiment, the drying agent can comprise at least one of crosscarmellose (e.g. Ac-Di-Sol), sodium polyacrylate, and sodium starch glycolate (e.g., Primojel). As previously noted, the oral dosage form may contain two or more drying agents as a part of the drying composition. The drying agents may be selected not only for their drying properties, but also for other enhancements in delivery of the active agent that they may provide. The drying composition may also optionally contain other additives and/or agents that enhance delivery of the active agent.

In one embodiment, the drying agent comprises a hydrogel polymer, such as a superporous hydrogel. Polymer hydrogels are cross-linked hydrophilic polymers that are capable of absorbing large amounts of water. Superporous hydrogels (SPH) may comprise polymer hydrogels that are capable of absorbing an amount of water in excess of 10 times their dry weight. In particular, the superporous hydrogels may have a three-dimensional cross-linked network containing large numbers of interconnected and open pores, that may allow for the absorption of significant quantities of water in a short period of time. Superporous hydrogels can be formed using various hydrophilic polymers, such as one or more of poly(acrylic acid-co-acrylamide) (poly(AA-co-AM), poly(AA-co-AM) coated with poly(ethyleneglycol-b-tetramethylene oxide, or grafted with poly(ethylene glocol), or semi or fully-interpenetrated with chitosan or polyethyleneimine, or sodium alginate, poly(acrylamide), poly(acrylic acid), glycol chitosan, polysaccharides, starches, and the like. In one embodiment, the super porous hydrogel comprises a polymer formed from cross-linking a hydrophilic polymer using a polycarboxylic acid as a cross-linking agent. For example, the hydrophilic polymer can comprise a polysaccharide such as a cellulose or cellulose derivative, such as an alkylcellulose (e.g. methylcellulose, ethycellulose and n-propylcellulose), substituted alkyl-celluloses (e.g., hydroxyethylcellulose, hydroxypropylmethylcellulose and carboxymethylcellulose), a hydroxycellulose, a starch or starch derivative, dextran, glycosaminoglycans, polyuronic acids, and the like. The polycarboxylic acid can comprise an organic acid having two or more carboxylic acid functional groups, such as dicarboxylic acids such as oxalic acid, malonic acid, maleic acid, malic acid, succinic acids, and the like, and tricarboxylic acids such as citric acid, isocitric acid, aconitic acid, phthalic acid, and the like. In one embodiment, the superporous hydrogel can comprise a hydrophilic polymer corresponding to carboxymethylcellulose cross-linked with citric acid, and/or a combination of hydrophilic polymers including carboxymethylcellulose and hydroxyethylcellulose cross-linked by citric acid, as described for example in U.S. Pat. Nos. 8,658,147, 9,353,191, and U.S. PG-Pub No. 2014/0296507, all of which are incorporated by reference herein in their entireties.

In one embodiment, the at least one drying agent comprises a sodium polyacrylate polymer having a fluid uptake capacity as measured by the Agent Fluid Uptake assay of at least 20 mg fluid/mg drying agent, such as at least 30 mg fluid/mg drying agent, and even at least 35 mg fluid/mg drying agent, and that may have less than 80 mg fluid/mg drying agent. A fluid uptake time with the sodium polyacrylate polymer as measured by the Agent Fluid Uptake time Assay may be less than 1 minute, such as less than 40 seconds, and even less than 35 seconds, such as no more than 30 seconds.

Permeation Enhancer

In yet another embodiment, the oral dosage form comprises at least one permeation enhancer to enhance permeation of the active agent through the intestinal tissue. In some embodiments, the permeation enhancer may be capable of opening a tight junction between cells (e.g., intestinal cells or epithelial cells). A permeation enhancer may, in some instances, facilitate uptake of an agent into epithelial cells. Representative classes of permeation enhancers include, but are not limited to, a fatty acid, a medium chain glyceride, a surfactant, a steroidal detergent, an acyl carnitine, lauroyl carnitine, palmitoyl carnitine, an alkanoyl choline, an N-acetylated amino acid, esters, salts, bile salts, sodium salts, nitrogen-containing rings, derivatives thereof, and combinations thereof. The permeation enhancer may be anionic, cationic, zwitterionic, or nonionic. Anionic permeation enhancers include, but are not limited to, sodium lauryl sulfate, sodium decyl sulfate, sodium octyl sulfate, N-lauryl sarcosinate, and sodium carparate. Cationic permeation enhancers include, but are not limited to, cetyltrimethyl ammonium bromide, decyltrimethyl ammonium bromide, benzyldimethyldodecyl ammonium chloride, myristyltrimethylammonium chloride, and dodecylpyridinium chloride. Zwitterionic permeation enhancers include, but are not limited to, N-dodecyl-N,N-dimethyl-3-ammonio-1-propanesulfonate, 3-(N,N-dimethylpalmitylammonio)propanesulfonate. Fatty acids include, but are not limited to, butyric, caproic, caprylic, pelargonic, capric, lauric, myristic, palmitic, stearic, arachidic, oleic, linoleic, and linolinic acid, salts thereof, derivatives thereof, and combinations thereof. In some embodiments, a fatty acid may be modified as an ester, for example, a glyceride, a monoglyceride, a diglyceride, or a triglyceride. Bile acids or salts including conjugated or unconjugated bile acid permeation enhancers include, but are not limited to, cholate, deoxycholate, tauro-cholate, glycocholate, taurodexycholate, ursodeoxycholate, tauroursodeoxycholate, chenodeoxycholate, derivates thereof, salts thereof, and combinations thereof. In some embodiments, permeation enhancers include a metal chelator, such as EDTA or EGTA, a surfactant such as sodium dodecyl sulfate, polyethylene ethers or esters, polyethylene glycol-12 lauryl ether, salicylate polysorbate 80, nonylphenoxypolyoxyethylene, dioctyl sodium sulfosuccinate, saponin, palmitoyl carnitine, lauroyl-1-carnitine, dodecyl maltoside, acyl carnitines, alkanoyl cjolline, and combinations thereof. Other permeation enhancers include, but are not limited to, 3-nitrobenzoate, zoonula occulden toxin, fatty acid ester of lactic acid salts, glycyrrhizic acid salt, hydroxyl beta-cyclodextrin, N-acetylated amino acids such as sodium N-[8-(2-hydroxybenzoyl)amino]caprylate and chitosan, micelle forming agents, passageway forming agents, agents that modify the micelle forming agent, agents that modify the passageway forming agents, salts thereof, derivatives thereof, and combinations thereof. In some embodiments, micelle forming agents include bile salts. In some embodiments, passageway forming agents include antimicrobial peptides. In some embodiments, agents that modify the micelle forming agents include agents that change the critical micelle concentration of the micelle forming agents. An exemplary permeation enhancer is 1% by weight 3-(N,N-dimethylpalmitylammonio)propanesulfonate. Permeation enhancers are also described in patent application publication US 2013/0274352, the contents of which are incorporated in their entirety herein. In one embodiment, the permeation enhancers can comprise at least one of EDTA, palmitoyl carnitine, lauroyl carnitine, dimethyl palmitoyl ammonio propanesulfonate (PPS), and sodium caprate.

In one embodiment, permeation enhancers selected for the oral dosage form may be selected on the basis of one or more of the predominant permeation mechanism and the hydrophilicity and/or hydrophobicity of the permeation enhancer. For example, permeation enhancers that are fatty esters and/or permeation enhancers having nitrogen-containing rings may exhibit more paracellular transport activity, whereas cationic and zwitterionic permeation enhancers may exhibit more transcellular activity, as described for example in the article to Whitehead and Mitragotri entitled “Mechanistic Analysis of Chemical Permeation Enhancers for Oral Drug Delivery” in Pharmaceutical Research, Vol. 25, No. 6, June 2008, pages 1412-1419, which is hereby incorporated by reference herein in its entirety. Furthermore, for those permeation enhancers having a transcellular mechanism, increases in hydrophobicity of the permeation enhancer may enhance this mechanism, whereas for permeation enhancers having more paracellular transport activity, greater enhancement may be seen for those permeation enhancers that are more hydrophillic (such as by interacting with hydrophilic constituents of tight junctions). In one embodiment the relative hydrophobicity/hydrophilicity of the enhancer may be determined by its log P value, with P being the octanol/water partition coefficient for the compound. For example, in one embodiment, to enhance transcellular transport, a permeation enhancer may have a log P value of at least 2, such as at least 4, and even at least 6. Conversely, to enhance paracellular transport, a permeation enhancer may in one embodiment have a log P of less than about 4, such as less than 2, and even less than 0.

A content of the permeation enhancer in the oral dosage form in one embodiment may be at least about 0.01% by weight, such as at least about 0.1% by weight, and no more than about 80% by weight, and may even be less than about 30% by weight. For example, in one embodiment, the content of permeation enhancer in the oral dosage form may be at least about 0.01% by weight, such as at least about 0.1 by weight, including at least about 1% by weight, such as at least about 5% by weight, and even at least about 10% by weight, such as at least about 30% by weight, or even at least about 50% by weight, such as at least about 70% by weight. For example, in one embodiment, the content of permeation enhancer may be in the range of from 0.1 by weight to 70% by weight, such as from about 0.1% by weight to about 20% by weight, and even from about 1% by weight to about 10% by weight.

Without being limited by any particular theory, according to one aspect, it is believed that the “drying effect” provided by the at least one drying agent and/or drying composition, can impart synergistic effects in terms of enhanced bioavailability when provided in combination with a permeation enhancer. That is, the combination of the drying agent and permeation enhancer may, in certain embodiments, provide a greater than additive effect in the increase in bioavailability of an active agent being delivered by a dosage form having the combination of drying agent and active agent, over dosage forms having only the drying agent (without permeation enhancer) or only permeation enhancer (without drying agent). The synergistic effects may not only advantageously increase the overall bioavailability for a particular active agent, but may also allow for lower doses of permeation enhancer to be used that what otherwise might be necessary in the absence of drying agent, which may be advantageous particularly in a case where the permeation enhancer has a relatively high toxicity. In one embodiment, the permeation enhancer may be provided in a total dosage amount that is in the range of from 0.1 mg to 800 mg per dosage form, such as 0.1 mg to 600 mg per total dosage form, such as a dosage in the range of from 1 mg to 200 mg, and even in a dosage in the range of from 10 mg to 40 mg per total dosage form. In one embodiment, the permeation enhancer is provided in a range of at least 5 mg to no more than 50 mg per dosage form, such as at least 15 mg to no more than 35 mg per dosage form. In another embodiment, the permeation enhancer is provided in a range of at least 50 mg to no more than 200 mg per dosage for, such as at least 75 mg to no more than 100 mg per dosage form. For example, the dosage form may have the permeation enhancer in a content of at least 0.1 mg per dosage form, such as at least 1 mg per dosage form, and even at least 10 mg per dosage form, such as at least 30 mg per dosage form, at least 50 mg per dosage form, and even larger values such as at least 100 mg per dosage form, at least 200 mg per dosage form, at least 400 mg per dosage form, and at least 600 mg per dosage form. In one embodiment, the dosage of the permeation enhancer will not exceed 600 mg for the dosage form, and may even be less than 400 mg, such as less than 200 mg, and even less than 100 mg, such as less than 50 mg, and even less than 30 mg. For example, for a permeation enhancer which may have relatively high toxicities compared to other permeation enhancers, a suitable dosage amount may be in the range of from 0.1 mg to 50 mg, such as from 1 mg to 50 mg, and even from 10 mg to 30 mg, for the total dosage form. In contrast, for a permeation which may have relatively low and/or average toxicities as compared to other permeation enhancers, a suitable dosage amount may be one of the higher dosages described above. In one embodiment, a permeation enhancer comprising sodium caprate is provided in an amount of at least 10 mg and no more than 50 mg per dosage form. In another embodiment, a permeation enhancer comprising PPS is provided in an amount of at least 10 mg and no more than 50 mg per dosage form.

Gelling Agent

According to one embodiment, the oral dosage form comprises a gelling agent that is capable of forming a gel upon exposure to an intestinal environment. In particular, in one embodiment, the gelling agent is exposed to intestinal fluids upon dissolution of a protective coating or other outer layer, thereby causing the gelling agent to thicken and form a viscous gel material. Without being limited to any particular theory, it is believed that including the gelling agent in the oral dosage form can improve delivery of the active agent by forming a thickened and semi-coherent mass with the active agent and/or drying agent upon exposure to the intestinal environment. The gelling agent may thus, in certain embodiments, improve delivery of an active agent in conjunction with the delivery of the drying agent, as well as improve retention of the active agent and/or drying agent adjacent intestinal tissue.

The gelling agent according to one embodiment comprises an agent that is capable of providing a gelling and/or thickening effect to a liquid, such as in an intestinal fluid. Suitable gelling agents can include at least one of pectin, hydroxypropylmethylcellulose (HPMC), acrylic acid polymer and copolymers, including carbopol polymers (such as CARBOPOL 934 P), acacia, alginic acid, polyvinyl alcohol, sodium alginate, tragacanth, methylcellulose, poloxamers, carboxymethyl cellulose, and ethyl cellulose. In one embodiment, the gelling agent comprises at least one of pectin, HPMC, and a carbopol polymer (e.g., CARBOPOL 934 P). Furthermore, in one embodiment a component that acts in concert with the gelling agent can be provided with the gelling agent to enhance gel formation. For example, in a case where pectin is used as a gelling agent, sucrose may also be provided to enhance gel formation by the pectin gelling agent. Other components that assist in gel formation, such as for example at least one of sucrose, mannitol, and fructose, may also be provided in combination with pectin or other gelling agent to provide for gel formation.

A content of the gelling agent in the oral dosage form in one embodiment can be selected according to the extent of gelling and/or thickening to be provided, as well as the structure and configuration of the oral dosage form. In one embodiment, the oral dosage form has at least about 1% by weight of a gelling agent. By way of further example, in one embodiment the oral dosage form has at least about 5% by weight of a gelling agent. By way of further example, in one embodiment the oral dosage form has at least about 10% by weight of a gelling agent. By way of further example, in one embodiment the oral dosage form has at least about 30% by weight of a gelling agent. In general, the content of the gelling agent in the oral dosage form will be less than about 50% by weight. By way of further example, in one embodiment the oral dosage form has a content of the gelling agent of less than 30% by weight. By way of further example, in one embodiment the oral dosage form has a content of the gelling agent of less than 10% by weight. For example, a content of gelling agent in the oral dosage form may be from about 1% by weight to about 50% by weight, such as from about 5% by weight to about 25% by weight, and even about 10% by weight to about 20% by weight. Furthermore, in one embodiment the oral dosage form is substantially absent any gelling agent, and thus may have an amount of gelling agent that is less than about 1% by weight, such as zero gelling agent in the composition.

The gelling agent may be provided in the oral dosage form in a configuration selected to provide gelling of a component of the oral dosage form upon exposure to the intestinal environment. For example, in one embodiment, the gelling agent can be provided intermixed with, or otherwise adjacent to, an active agent. In another embodiment, the gelling agent can be provided intermixed with, or otherwise adjacent to, a drying agent. In one embodiment, the gelling agent is provided in a region that surrounds a region having the at least one drying agent therein. In one embodiment, the at least one drying agent is provided in a region that surrounds a region having the gelling agent therein. In one embodiment, the gelling agent is provided in a same layer with at least one of the active agent and drying agent, and/or the gelling agent may be provided in a layer immediately adjacent to a layer containing at least one of the active agent and drying agent. The gelling agent may also be intermixed with at least one of the active agent and/or drying agent, so as to form a homogenous layer and/or phase in the oral dosage formulation, or in a case where a gelling agent is provided in a particulate form, the gelling agent particles may be combined with particles of at least one of the active agent and/or drying agent to form a mixture suitable for the oral dosage form. Furthermore, other configurations of the gelling agent in the oral dosage form, such as in different layers or other combinations with the active agent and/or drying agent, can also be provided.

Other Additives

The oral dosage form can comprise further additives in addition to the active agent and at least one drying agent, and alternatively or in addition to the gelling agent, that further enhance delivery of the active agent. For example, in one embodiment, the oral dosage form comprises an osmagent that assists in delivery of the active agent. Without being limited by any one theory, it is believed that the osmagent may assist in expelling the active agent from the oral dosage form, by absorbing water and pushing the active agent from the oral dosage form, and/or may help to open tight junctions in the intestine by pulling water therefrom. In one embodiment, an osmagent capable of being hydrated may include water-soluble salts, carbohydrates, small molecules, amino acids, water-soluble hydrogel forming polymers, and combinations thereof. Exemplary water-soluble salts may include, without limitation, magnesium chloride, magnesium sulfate, lithium chloride, sodium chloride, potassium chloride, lithium sulfate, sodium sulfate, potassium sulfate, sodium hydrogen phosphate, potassium hydrogen phosphate, sodium acetate, potassium acetate, magnesium succinate, sodium benzoate, sodium citrate, sodium ascorbate, and the like, and combinations thereof. Exemplary carbohydrates may include sugars such as arabinose, ribose, xylose, glucose, fructose, galactose, mannose, sucrose, maltose, lactose, raffinose, and the like, and combinations thereof. Exemplary amino acids may include glycine, leucine, alanine, methionine, and the like, and combinations thereof. Exemplary water-soluble hydrogel forming polymers may include sodium carboxy methylcellulose, hydroxypropyl methylcellulose (HPMC), hydroxyethyl methylcellulose, crosslinked PVP, polyethylene oxide, carbopols, polyacrylamindes, and the like, and combinations thereof. In one embodiment, the osmagent provided in the oral dosage form comprises at least one of sucrose, mannitol, fructose and polyethylene glycol. A content of the osmagent in the oral dosage form in one embodiment may be at least about 1% by weight, and less than about 60% by weight, such as from about 10% by weight to about 50% by weight, and even from about 20% by weight to about 40% by weight.

In one embodiment, the oral dosage form can comprise one or more controlled release/extended release agents, typically in the form of a polymeric material that is capable of forming a matrix about the active agent upon exposure to fluid, to slow release of the active agent from the dosage form. For example, the dosage form can comprise one or more the gelling agents described above as a controlled release/extended release agent. For example, the controlled release/extended release agent can comprise one or more of pectin, hydroxypropylmethylcellulose (HPMC), acrylic acid polymer and copolymers, including carbopol polymers (such as CARBOPOL 934 P), acacia, alginic acid, polyvinyl alcohol, sodium alginate, tragacanth, methylcellulose, poloxamers, carboxymethyl cellulose, and ethyl cellulose. In one embodiment, the controlled release/extended release agent comprises hydroxypropyl methyl cellulose (HPMC) as a controlled release/extended release agent. The controlled release/extended release agent can be incorporated into one or more active agent regions 105 of the dosage form that contain the at least one active agent, such as for example in either tablet or capsule form.

Other additives and/or excipients that can be provided as a part of the oral dosage form can include one or more of stabilizers, glidants, bulking agents, anti-adherents, mucoadhesive agents, binders, sorbents, preservatives, cryoprotectants, hydrating agents, enzyme inhibitors, mucus modifying agents (e.g., mucus drying agents, etc.), pH modifying agents, solubilizers, plasticizers, crystallization inhibitors, bulk filling agents, bioavailability enhancers, and combinations thereof. In some embodiments, the additives and/or excipients may include polyethylene glycols, polyethylene oxides, humectants, vegetable oils, medium chain mono, di-, and triglycerides, lecithin, waxes, hydrogenated vegetable oils, colloidal silicon dioxide, polyvinylpyrrolidone (PVP) (“povidone”), celluloses, CARBOPOL® polymers (Lubrizol Advanced Materials, Inc.) (i.e., crosslinked acrylic acid-based polymers), acrylate polymers, pectin, sugars, magnesium sulfate, or other hydrogel forming polymers.

Protective Coating

The oral dosage form according to one embodiment further comprises a protective coating that at least partially protects the oral dosage form from the acidic environment in the stomach to deliver the active agent to a region of the intestine. The protective coating can, in one embodiment, form an outer coating of the oral dosage form that protects the active agent and/or drying agents or other agents inside the oral dosage form. While in one embodiment the protective coating completely covers an outer surface of the oral dosage form, the protective coating may also optionally be devised to cover only a portion of the outer surface of the oral dosage form. The protective coating can also comprise only a single coating layer, or can be configured as multiple coating layers.

According to one embodiment, the protective coating may be an enteric coating that is a pH dependent coating, having an enteric material that is a polymer that is substantially insoluble in the acidic environment of the stomach, but that has increased solubility in intestinal fluids that are at a higher pH. That is, the enteric coating may preferentially dissolve and/or become at least partially permeable in the intestine as opposed to in the stomach. For example, the enteric coating may be formed of an enteric material that is substantially insoluble at a pH below about 5, such as in the acidic environment of the stomach, but that becomes soluble at higher pH, such as a pH of at least about 5.5 for the duodenum, a pH of at least about 6.5 for the jejunum, and a pH of at least about 7.0, such as at least about 7.5 for the ileum (the duodenum, jejunum and ileum are part of the small intestine). That is, the enteric coating can be selected to be insoluble at lower pH, but soluble at a higher pH, such that the enteric coating can be made to dissolve and/or become at least partially permeable and release the contents of the oral dosage form once an environment of the gastrointestinal system is reached having a pH in which the material of the enteric coating is soluble. Accordingly, suitable enteric materials for forming the enteric coating in one embodiment are those that are not soluble until a pH of at least about 5.5 is reached, such as a pH of at least about 6.0. In one embodiment, suitable enteric materials for forming the enteric coating in one embodiment are those that are not soluble until a pH of at least about 6.5 is reached, such as a pH of at least about 7.0, and even a pH of at least about 7.5. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, poly(vinylalcohol), natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, Kollicoat MAE 100P and Aquateric). For example, in one embodiment the enteric materials used to form the enteric coating can comprise at least one of Eudragit S100 (poly(methacrylic acid-co-methyl methacrylate) 1:2), Eudragit L100 (poly(methacrylic acid-co-methyl methacrylate) 1:1), and Kollicoat MAE 100P (methacrylic acid ethyl acrylate copolymer 1:1). The solubility of each of the above materials at a specific pH is either known or is readily determinable in vitro. For example, the foregoing is a list of possible materials, but one of skill in the art with the benefit of the instant disclosure would recognize that the foregoing list is not comprehensive and that there are other enteric materials that may be used. In yet another embodiment, the protective coating may be one that dissolved and/or becomes partially permeable due to a change in environment that is unrelated to pH. Furthermore, in another embodiment, the protective and/or enteric coating may be one that dissolves and/or becomes at least partially permeable at a predetermined rate as it passes through the gastrointestinal system, to provide a controlled and/or timed release of the active agent at a predetermined region of the intestine.

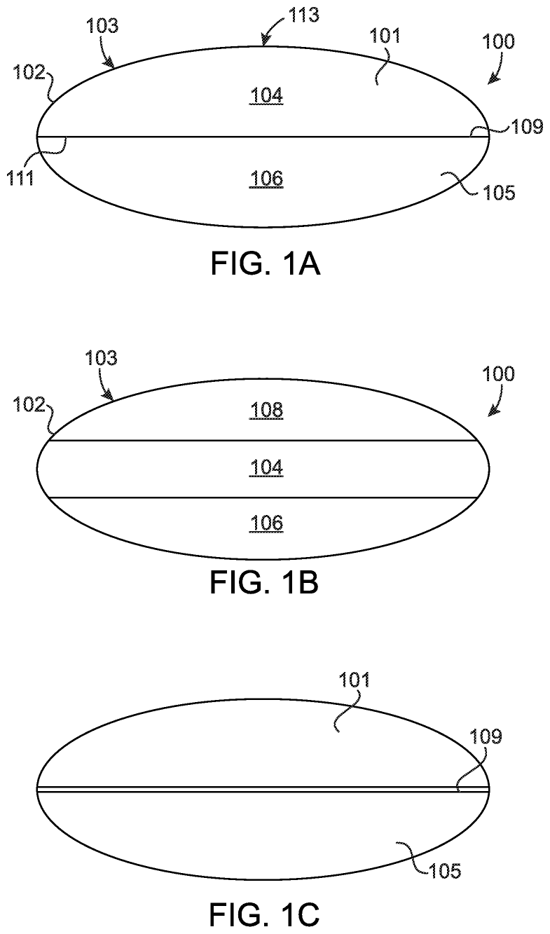

In one embodiment, the protective coating comprises at least a portion thereof that becomes permeable and/or dissolves under predetermined conditions, such as at a predetermined pH (e.g., a pH at a targeted site of the intestine), or following exposure to fluid for a pre-determined period of time (e.g., controlled release following administration at a predetermined point in time). In one embodiment, the protective coating substantially entirely comprises a coating of a material that becomes permeable and/or dissolved under the predetermined conditions. According to yet another embodiment, the protective coating can comprise a first coating region that becomes permeable and/or dissolved under predetermined conditions, and a second coating region that substantially does not become permeable and/or does not dissolve under the predetermined conditions, and/or that becomes permeable and/or dissolves to a lesser extent than the first coating region. Such first and second coating regions may be provided, for example, in embodiments where different regions of the dosage form are to be released at different points in time and/or at different rates. For example, a first coating region may be provided to at least partially coat a section of the dosage form containing one of the active agent or drying agent, whereas as second coating region may be provided to at least partially coat a section of the dosage form containing the other of the active agent or drying agent, to provide different rates of release of the agents. In yet another embodiment, the protective coating comprises the first coating region that becomes permeable and/or dissolves under the predetermined conditions, as a major portion of the protective coating. For example, first coating region may be provided as a part of the protective coating such that it covers at least 25% and even at least 35% of the surface of the dosage form, such as at least 40%, and even at least 50%, such as at least 60% and even 75%, such as at least 90% of the surface of the dosage form. In yet another embodiment, the first coating region that becomes at least partially permeable and/or dissolves under the predetermined conditions may cover at least 25% and even at least 35% of a surface of a region of the oral dosage form containing the drying agent, such as at least 40% and even at least 50%, include at least 60% and even at least 75%, such as at least 90% of the surface of the region. For example, in the case where the dosage form is in the form of a tablet having a layer 104 containing the at least one drying agent therein, as shown for example in FIG. 1A, the protective coating 103 may substantially entirely comprise a material that becomes permeable and/or dissolves at the predetermined conditions about substantially the entire surface 113 of the dosage form, and/or may comprise the material that becomes permeable and/or dissolves at the predetermined conditions about at least a surface 113 of the dosage form corresponding to a surface of the region 101 containing the drying agent. In one embodiment, by providing a protective coating having a permeable and/or dissolving portion that surrounds a majority of the surface of the dosage form, the contents of the dosage form can be effectively released, and in a multi-directional manner, without unnecessarily retaining contents inside the dosage form. Furthermore, in yet another embodiment, by providing the permeable and/or dissolving portion about a majority of at least the surface of a region of the dosage form containing the drying agent, good release of the drying agent from a relatively large surface region of the dosage form can be provided.

The protective coating is formed on the surface of the oral dosage form according to a suitable method. In one embodiment, the protective coating is formed by spray coating materials such as enteric materials onto the surface of the oral dosage form, until a coating having a thickness within a predetermined range has been formed. The protective material may, in one embodiment, be sprayed relatively uniformly on the oral dosage form to provide a protective coating having a uniform thickness on the surface of the oral dosage form. The protective coating may also, in another embodiment, be sprayed non-uniformly, according to a configuration of the oral dosage form and the desired release characteristics. In yet another embodiment, the protective coating can be formed on the surface of the oral dosage form by a dip-coating method, where the surface of the oral dosage form is dipped or otherwise immersed in a fluid containing the protective coating materials, such as enteric coating materials, to form a coating of the protective materials on the surface. In one embodiment, a thickness of the protective coating formed on the surface of the oral dosage form is correlated with a percent weight gain of the coated oral dosage form as compared to the uncoated form, and thus a thickness of the protective coating within a predetermined range can be achieved by coating with the protective coating materials until a percent weight gain is obtained that is within a predetermined range. For example, in one embodiment, a protective coating may be formed on the surface to provide a coating having at least about 2 mg coating/cm² tablet surface. In another embodiment, the protective coating may be formed on the surface to provide a coating having at least about 4 mg coating/cm² tablet surface. In another embodiment, the protective coating may be formed on the surface to provide a coating having at least about 8 mg coating/cm² tablet surface. In another embodiment, the protective coating may be formed on the surface to provide a coating having at least about 12 mg coating/cm² tablet surface. In another embodiment, the protective coating may be formed on the surface to provide a coating having at least about 20 mg coating/cm² tablet surface. In general, the protective coating will be less than about 80 mg coating/cm² tablet surface. By way of example, the weight gain resulting from formation of the protective coating may be in the range of from about 2 mg coating/cm² tablet surface to about 80 mg coating/cm² tablet surface, such as from about 4 mg coating/cm² tablet surface to about 20 mg coating/cm² tablet surface, and even from about 6 mg coating/cm² tablet surface to about 16 mg coating/cm² tablet surface.

In some embodiments, the oral dosage form may be configured for controlled release of the active agent at a region in the intestine, for example by providing a protective coating corresponding to an enteric coating that provides for controlled release at a predetermined pH and/or pH range. Additionally and/or alternatively, other ingredients and/or excipients may be provided in the oral dosage form to provide for a controlled release of the active agent and/or drying agent. In addition to the protective coating, the overall architecture of the dosage form, such as for example the structure and arrangement of the drying agent region with respect to the active agent region, the level of compression of the dosage form (if compressed), and composition of components of the dosage form can also be selected to provide a predetermined release of the active agent from the dosage form.

For example, in one embodiment, a release rate for the agent may be at least about 90% within 1 min, as determined by USP Dissolution Assay 711 with Apparatus 1 and a dissolution medium of 150 mM phosphate buffered saline at a pH of 5.0. By way of further example, in one embodiment, a release rate for the agent may be at least about 90% within 1 min, as determined by USP Dissolution Assay 711 with Apparatus 1 and a dissolution medium of 150 mM phosphate buffered saline at a pH of 5.5. By way of further example, in one embodiment, a release rate for the agent may be at least about 90% within 1 min, as determined by USP Dissolution Assay 711 with Apparatus 1 and a dissolution medium of 150 mM phosphate buffered saline at a pH of 6.0. By way of further example, in one embodiment, a release rate for the agent may be at least about 90% within 1 min, as determined by USP Dissolution Assay 711 with Apparatus 1 and a dissolution medium of 150 mM phosphate buffered saline at a pH of 6.5. By way of further example, in one embodiment, a release rate for the agent may be at least about 90% within 1 min, as determined by USP Dissolution Assay 711 with Apparatus 1 and a dissolution medium of 150 mM phosphate buffered saline at a pH of 7.0. By way of further example, in one embodiment, a release rate for the agent may be at least about 90% within 1 min, as determined by USP Dissolution Assay 711 with Apparatus 1 and a dissolution medium of 150 mM phosphate buffered saline at a pH of 7.5.

For example, in one embodiment, a release rate for the agent may be at least about 90% within 10 min, as determined by USP Dissolution Assay 711 with Apparatus 1 and a dissolution medium of 150 mM phosphate buffered saline at a pH of 5.0. By way of further example, in one embodiment, a release rate for the agent may be at least about 90% within 10 min, as determined by USP Dissolution Assay 711 with Apparatus 1 and a dissolution medium of 150 mM phosphate buffered saline at a pH of 5.5. By way of further example, in one embodiment, a release rate for the agent may be at least about 90% within 10 min, as determined by USP Dissolution Assay 711 with Apparatus 1 and a dissolution medium of 150 mM phosphate buffered saline at a pH of 6.0. By way of further example, in one embodiment, a release rate for the agent may be at least about 90% within 10 min, as determined by USP Dissolution Assay 711 with Apparatus 1 and a dissolution medium of 150 mM phosphate buffered saline at a pH of 6.5. By way of further example, in one embodiment, a release rate for the agent may be at least about 90% within 10 min, as determined by USP Dissolution Assay 711 with Apparatus 1 and a dissolution medium of 150 mM phosphate buffered saline at a pH of 7.0. By way of further example, in one embodiment, a release rate for the agent may be at least about 90% within 10 min, as determined by USP Dissolution Assay 711 with Apparatus 1 and a dissolution medium of 150 mM phosphate buffered saline at a pH of 7.5.

For example, in one embodiment, a release rate for the agent may be at least about 90% within 5 min, as determined by USP Dissolution Assay 711 with Apparatus 1 and a dissolution medium of 150 mM phosphate buffered saline at a pH of 5.0. By way of further example, in one embodiment, a release rate for the agent may be at least about 90% within 5 min, as determined by USP Dissolution Assay 711 with Apparatus 1 and a dissolution medium of 150 mM phosphate buffered saline at a pH of 5.5. By way of further example, in one embodiment, a release rate for the agent may be at least about 90% within 5 min, as determined by USP Dissolution Assay 711 with Apparatus 1 and a dissolution medium of 150 mM phosphate buffered saline at a pH of 6.0. By way of further example, in one embodiment, a release rate for the agent may be at least about 90% within 5 min, as determined by USP Dissolution Assay 711 with Apparatus 1 and a dissolution medium of 150 mM phosphate buffered saline at a pH of 6.5. By way of further example, in one embodiment, a release rate for the agent may be at least about 90% within 5 min, as determined by USP Dissolution Assay 711 with Apparatus 1 and a dissolution medium of 150 mM phosphate buffered saline at a pH of 7.0. By way of further example, in one embodiment, a release rate for the agent may be at least about 90% within 5 min, as determined by USP Dissolution Assay 711 with Apparatus 1 and a dissolution medium of 150 mM phosphate buffered saline at a pH of 7.5. In yet another embodiment, a release rate for the agent may be at least about 90% within 30 min, as determined by USP Dissolution Assay 711 with Apparatus 1 and a dissolution medium of 150 mM phosphate buffered saline at a pH of 5.0. By way of further example, in one embodiment, a release rate for the agent may be at least about 90% within 30 min, as determined by USP Dissolution Assay 711 with Apparatus 1 and a dissolution medium of 150 mM phosphate buffered saline at a pH of 5.5. By way of further example, in one embodiment, a release rate for the agent may be at least about 90% within 30 min, as determined by USP Dissolution Assay 711 with Apparatus 1 and a dissolution medium of 150 mM phosphate buffered saline at a pH of 6.0. By way of further example, in one embodiment, a release rate for the agent may be at least about 90% within 30 min, as determined by USP Dissolution Assay 711 with Apparatus 1 and a dissolution medium of 150 mM phosphate buffered saline at a pH of 6.5. By way of further example, in one embodiment, a release rate for the agent may be at least about 90% within 30 min, as determined by USP Dissolution Assay 711 with Apparatus 1 and a dissolution medium of 150 mM phosphate buffered saline at a pH of 7.0. By way of further example, in one embodiment, a release rate for the agent may be at least about 90% within 30 min, as determined by USP Dissolution Assay 711 with Apparatus 1 and a dissolution medium of 150 mM phosphate buffered saline at a pH of 7.5. In yet another embodiment, a release rate for the agent may be at least about 90% within 2 hours, as determined by USP Dissolution Assay 711 with Apparatus 1 and a dissolution medium of 150 mM phosphate buffered saline at a pH of 5.0. By way of further example, in one embodiment, a release rate for the agent may be at least about 90% within 2 hours, as determined by USP Dissolution Assay 711 with Apparatus 1 and a dissolution medium of 150 mM phosphate buffered saline at a pH of 5.5. By way of further example, in one embodiment, a release rate for the agent may be at least about 90% within 2 hours, as determined by USP Dissolution Assay 711 with Apparatus 1 and a dissolution medium of 150 mM phosphate buffered saline at a pH of 6.0. By way of further example, in one embodiment, a release rate for the agent may be at least about 90% within 2 hours, as determined by USP Dissolution Assay 711 with Apparatus 1 and a dissolution medium of 150 mM phosphate buffered saline at a pH of 6.5. By way of further example, in one embodiment, a release rate for the agent may be at least about 90% within 2 hours, as determined by USP Dissolution Assay 711 with Apparatus 1 and a dissolution medium of 150 mM phosphate buffered saline at a pH of 7.0. By way of further example, in one embodiment, a release rate for the agent may be at least about 90% within 2 hours, as determined by USP Dissolution Assay 711 with Apparatus 1 and a dissolution medium of 150 mM phosphate buffered saline at a pH of 7.5.

The oral dosage form may also be configured to provide different layers or structures therein having the active agent, drying agent and/or other excipients therein, that provide different rates of release of the active agent and/or drying agent from the oral dosage form. For example, in one embodiment the oral dosage form may have a first rate of release of at least one of the active agent and drying agent from a first part of the oral dosage form (e.g., a first layer or section of the oral dosage form), and may have a second rate of release of at least one of the active agent and drying agent from a second part of the oral dosage form (e.g., a second layer of section of the oral dosage form), that is different from the first rate of release.

Oral Dosage Form

The oral dosage form having the active agent and at least one drying agent can be provided in a variety of suitable dosage forms, including tablet forms, capsules, caplets, and combinations thereof. The active agent, at least one drying agent, and optionally other pharmaceutically acceptable excipients can be combined in different layers and forms, such as particulate forms, to provide for delivery of the active agent to the region of the intestine, and enhanced bioavailability of the delivered active agent.

According to one embodiment, the oral dosage form 100 comprises a first region 101 comprising the at least one drying agent, and a second region 105 comprising the at least one active agent. For example, as shown in the embodiment FIG. 1A, an oral dosage form 100 can comprise one or more drying agent regions 101 capable of drying an area about the intestinal site, and one or more active agent regions 105 comprising an active agent to be delivered to the intestinal site. The one or more drying agent regions 101 may be physically separated from the one or more active agent regions, for example to allow the drying agent to dry in an area about the intestinal site, without excessively inhibiting release of the active agent from the formulation. That is, without being limited by any particular theory, it is believed that certain drying agents may tend to form an at least partially coherent mass upon uptake of fluid, which mass can in turn interfere with access of active agent to the intestinal tissue site, for example by absorbing active agent into the mass of drying agent, or by otherwise presenting a physical barrier to access of the active agent to the intestinal tissue site. Accordingly, physically separating the drying agent from the active agent, into separate regions 101,105 of the oral dosage form 100, may promote the enhancement in bioavailability provided by the drying effect. In the embodiment show in FIG. 1A, the first and second regions 101 and 105 comprise separate layers 104, 106 of a tablet. However, other configurations of the first and second regions 101, 105 can also be provided, such as for example first and second regions in the form of separate elements, such as separate mini-tabs or mini-wafers contained an oral dosage form, and/or separate compartments or separate phases of materials, and even separate regions of beads and/or particles in capsule form. Other configurations where separate regions are provided for the active agent and drying agent can also be provided. Furthermore, while the embodiment depicted in FIG. 1A shows just a single active agent region 105 and a single drying agent region 101 (i.e., corresponding to the tablet layers 104, 106), certain embodiments can also include a plurality of each of the regions 101,105, according to the drying and/or active agent delivery requirements of the oral dosage form 100.

In one embodiment, the oral dosage form comprises a discrete boundary 109 between the one or more drying agent regions 101 and active agent regions 105. For example, in the embodiment as shown in FIG. 1A, the layers 104, 106 have a discrete boundary 109 therebetween that separates the composition of the first layer 104 from that of the second layer 106. In one embodiment, the discrete boundary 109 may simply be the interface between regions 101, 105 having different compositions. However, in yet another embodiment, the discrete boundary 109 may comprise a barrier layer 111 or other barrier element that separates the regions 101, 105. For example, the barrier layer 111 may inhibit and even prevent diffusion of the compositions in each region 101, 105 into one another, and may even act to at least partially separate and inhibit diffusion of the region contents upon dissolution of the dosage form 100 in the intestinal tract. A suitable barrier layer 111 may be formed of a material that at least partially impermeable to diffusion of the active agent and drying agent region contents therethrough, such as for example a polymeric material and/or a material selected from the group consisting of high density polyethylenes, waxes (e.g. beeswax), and rubbers. Examples of barrier materials may be described, for example, in the article “A Review on Controlled Porosity Osmotic Pump Tablets and Its Evaluation” by Sahoo et al, Bulletin of Faculty of Pharmacy, Cairo University, Volume 53, Issue 2, pages 195-205, December 2015, which is hereby incorporated by reference herein in its entirety. The barrier layer 111 may also comprise a thickness that is suitable to inhibit diffusion of the active agent and/or drying agent region contents therethrough, the thickness being related to a mass of the barrier layer material provided to form the layer on the dosage form. For example, the total weight of the barrier layer material provide to form the barrier layer on the dosage form may be at least 30 mg, such as at least 100 mg, and even at least 200 mg. In one embodiment, the barrier layer material weight per dosage form may be less than about 400 mg. For example, the barrier layer may be provided in a range of form 40 mg to 400 mg per dosage form, such as 50 mg to 150 mg per dosage form. According to one aspect, providing the barrier layer 111 may even serve to provide faster release of the active agent from the formulation, by reducing interaction of the active agent with the drying agent region.

In one embodiment, the dosage form can comprise a first region 105 having the at least one active agent to be delivered to the intestinal site, and a second region 101 having the at least one drying agent in a total drying agent percent by weight of at least 10 wt % of the second region. For example, the at least one drying agent may be provided in a total percent content of the second region 101 that is at least 20 wt %, and even at least 25 wt %, such as at least 30 wt %, and even at least 35 wt %. In one embodiment, the at least one drying agent may be provided in a total percent content of the second region that is at least 50 wt % and even at least 75 wt %, such as at least 80 wt %, and even at least 90 wt %, including at least 95 wt %, such as at least 99 wt %. For example, in one embodiment, the at least one drying agent may be provided in a total percent content of the second region that is in the range of from 50 wt % to 99 wt %, including in a range of from 80 wt % to 99 wt %.

According to one embodiment, the separate regions 101, 105 provide separate delivery of the active agent and/or drying agent, such that the active agent is provided substantially entirely in the active agent region 105, and the drying agent is provided substantially entirely in the drying agent region 101. For example, in one embodiment, the one or more active agent regions 105 comprise less than 30 wt % of drying agent and even less than 20 wt % of drying agent therein, such as less than 5 wt % of drying agent, and even less than 1 wt % of drying agent. For example, a ratio by weight of a total content of drying agent in the drying agent region 101 to a total content of drying agent in the active agent region 105 may be at least 20:1, such as at least 100:1, and even at least 1,000:1. As another example, the one or more drying agent regions 101 may comprise less than 20 wt %, such as less than 5 wt % of active agent therein, including less than 1 wt % of active agent, and even less than 0.1 wt % of active agent. For example, a ratio by weight of a total content of active agent in the active agent region 105 to a total content of active agent in the drying agent region 101 may be at least 20:1, such as at least 100:1, and even at least 1,000. Furthermore, while embodiments of the oral dosage form 100 are described as having the separate regions 101, 105, it should be understood that embodiments can also include oral dosage forms with at least one region where the active agent and/or drying agent are mixed together, such as uniformly or in a gradient mixture, either in combination with separate regions 101 and 105, or even dosage forms that are substantially without separation of the drying agent from the active agent into separate regions.

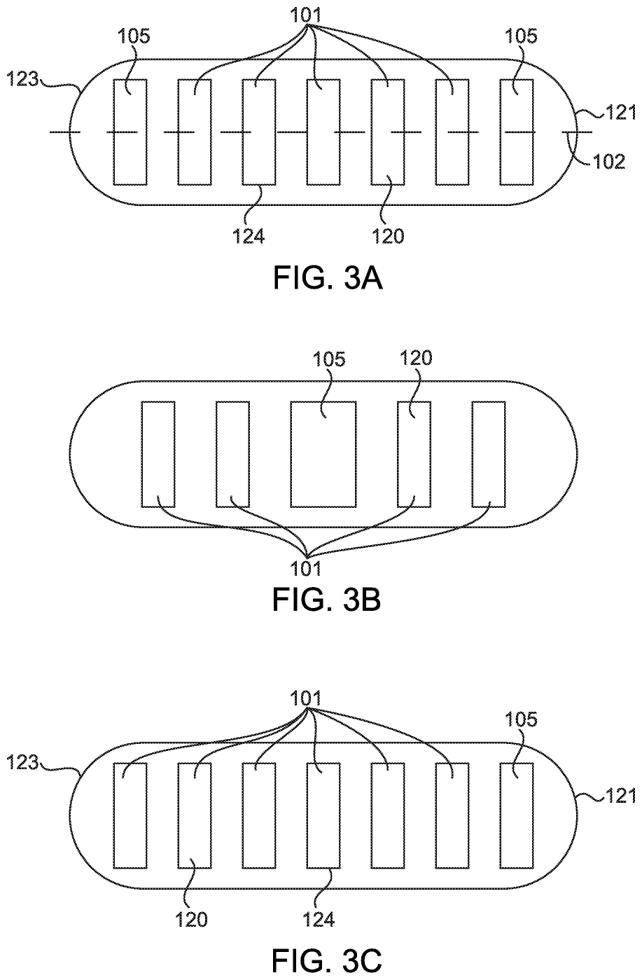

In the embodiment as shown in FIG. 1A, the active agent region 105 and drying agent region 101 comprise separate layers 104, 106, such as separate layers of an oral dosage form 100 comprising a tablet. However, the separate regions 101 and 105 may also be provided in different forms, as discussed above. In one embodiment, the drying agent region 101 and active agent region 105 are provided in separate structural elements 120 in the oral dosage form. For example, the oral dosage form 100 can comprise a drying agent region 101 comprising one or more elements 120 having the at least one drying agent therein. As another example, the oral dosage form 100 can comprise an active agent region 105 comprising one or more elements 120 having the at least one active agent therein. The one or more elements can comprise various shapes and forms suitable for delivery of the active agent and/or drying. For example, the one or more elements 120 can comprise at least one selected from the group consisting of tablets (e.g., mini-tabs), wafers, particles, granules, bulk polymeric matrices, and beads, as well as combinations thereof. The drying agent region may also comprise one or more of a layer tablet, particle, granule, bead, bulk polymeric material, and combinations thereof. The active agent region may also comprise one or more of a layer tablet, particle, granule, bead, lipophilic vehicle, emulsion, suspension, solution, semi-solid, liquid, and combinations thereof. FIGS. 3A-3C illustrate embodiments of a capsule having a plurality of elements 120 in the form of mini-tablets encapsulated by the capsule body. According to one aspect, the one or more elements 120 comprise a unitary structure having the active agent and/or drying agent therein. Alternatively and/or additionally, the dosage form can comprise a mixture of one or more elements having a unitary structure, combined with regions having non-unitary structures and even liquid or non-solid phases, as is discussed in more detail below. In yet another embodiment, the oral dosage form can comprise one or more elements 120 having a mixture of both active agent and drying agent therein (optionally with another component, such as a permeation enhancer).

According to yet another embodiment, the position of the one or more drying agent regions 101 and the one or more active agent regions 105 may be selected to provide enhanced delivery of the active agent, by simultaneously delivering the active agent while also providing a drying effect about the target intestinal site. For example, according to one embodiment, at least one active agent region 105 is provided at a region located at a periphery 123 of the oral dosage form 100, while at least one drying agent region 101 is provided at an interior region 124 of the oral dosage form. Alternatively, in another embodiment, at least one drying agent region 101 is provided at a region located at the periphery 123 of the oral dosage form, and at least one active agent region 105 is provided at the interior region of the oral dosage form 100. According to yet another embodiment, the oral dosage form can comprise both at least one drying agent region 101 and at least one active agent region 105 at the periphery 123 of the oral dosage form, such as an opposing ends or opposing faces of the oral dosage form. In yet another embodiment, the oral dosage form 100 can comprise both at least one drying agent region 101 and at least one active agent region 105 at the interior region 124 of the oral dosage form 100. In yet a further embodiment, at least one of the active agent region 105 and the drying agent region 101 may extend from a periphery 123 of the oral dosage form to an interior region 124 of the oral dosage form 100. For example, one of the regions 101, 105 may be located at at least one peripheral end of the dosage form, while the other region may extend from an opposing peripheral end to the interior region 124 of the oral dosage form.

According to one embodiment, as shown in FIG. 1A, at least one active agent region 105 and at least one drying agent region 101 are provided on opposing sides of the oral dosage form, such as by providing separate layers 104, 106 of a tablet formulation, the layers 104, 106 being on opposing sides from one another. According to yet another aspect, as shown in FIGS. 3A and 3C-3D, the least one active agent region 105 is be provided at a peripheral end 121 of the dosage form. For example, in the embodiments shown in FIGS. 3A and 3C, an element 120 in the form of a mini-tab comprising the active agent is provided at at least one peripheral end 121 of the dosage form 100 (FIGS. 3C-3D), and even at both opposing peripheral ends 121 (FIG. 3A). In yet another example, as shown in FIGS. 3B and 3C-3D, the at least one drying agent region 101 can be provided at a peripheral end 121 of the dosage form 100. For example, in the embodiment shown in FIGS. 3C-3D, elements 120 in the form of mini-tabs comprising the drying agent are provided at peripheral ends 121 of the dosage form. In the embodiment shown in FIG. 3B, the elements 120 in the form of mini-tabs are provided at opposing peripheral ends 121 of the dosage form, with an active agent region 101 disposed in between the peripheral drying agent regions. The active agent and drying agent regions can also be provided in an alternating form, such as for example by providing alternating layers 104, 106, 108 as in the embodiment shown in FIG. 1B, and/or for example by alternating active agent or drying agent-containing elements 120 along a longitudinal axis 122 of the dosage form. In one embodiment, active agent regions 105 are located at opposing peripheral ends 121 of the dosage form, with at least one drying agent region 101 provided between the active agent regions 105. In yet another embodiment, drying agent regions 101 are provided at opposing peripheral ends 121 of the dosage form, with at least one active agent region 105 provided between the drying agent regions 101. In yet another embodiment, the volume of the dosage form 100 occupied by the drying agent region 101 may be selected in relation to a volume of the dosage from occupied by the active agent region 105 to provide suitable effects. For example, in one embodiment, a ratio of the volume of the drying agent region to the volume of the active agent region may be in the range of from 10:1 to 0.1, such as from 8:1 to 1:1, and even from 4:1 to 2:1.

In yet another embodiment, at least one of the drying agent region 101 and active agent region 105 comprises a further additive, such as for example at least one of a permeation enhancer and/or gelling agent. In one embodiment, a permeation enhancer is provided as a part of the active agent region 105, to enhance permeation of the active agent at the intestinal site. The permeation enhancer may be provided in any of the amounts discussed above, and can comprise any of the permeation enhancers referred to above. The total amount of the permeation enhancer provided in the dosage form can be selected, for example, according to the type of permeation enhancer provided, and may be any of the amounts described above that may be suitable for the dosage from. In one further embodiment, the drying agent region 101 having the at least one drying agent has little or even substantially none of the permeation enhancer included therein, for example such that the drying effect can be provided substantially without inhibiting the effects of the permeation enhancer.

According to yet another embodiment, the dosage form 100 may be comprise a compressed form, such as a tablet or mini-tablet form, that is compressed to an extent that is sufficient to cohere the ingredients of the form for oral administration to a subject, while also providing for good hydration and release of the at least one drying agent from the oral dosage form 100. In particular, it has been discovered the compressing by applying a relatively low compression force and/or low compression pressure, so as to yield a compressed form having a relatively low density, may allow for fluid to more readily permeate the form to hydrate the at least one drying agent, while also providing for good release of the active agent from the dosage form. For example, in one embodiment, the compressed form (e.g., tablet or minitablet) may be compressed in a tablet press by applying a force of less than 4800 lbf, such as less than 3500 lbf, and even less than 3000 lbf, such as less than about 2500 lbf, and even less than about 2000 lbf. A mini-tablet that is provided as a structural element in a capsule may be compressed at even lower compression forces, as is further described below. In one embodiment, the compressed form may be compressed by applying a pressure of no more than 18,000 psi, such as a pressure of no more than 15000 psi, and even no more than 11000 psi, such as no more than 10,000 psi and even no more than 9,000 psi. In one embodiment, the compressed form will be compressed by applying a pressure of at least 5000 psi, and even at least 6600 psi, such as at least 8000 psi. For example, for a dosage form comprising a tablet, the dosage form may be compressed by applying at a pressure of at least 5000 psi and no more than 18,000 psi, such as a pressure of at least 6000 psi and no more than 15000, and even a pressure of at least 8000 psi and no more than 11000 psi. In a further embodiment, the compressed form may be compressed at a compression force and/or compressive pressure to provide a compressed form having a relatively lower density as measured in mg of the dosage form per volume of the dosage form. For example, the compressed form (e.g., tablet or minitablet) may have a density of no more than 1.11 mg/mm³, such as a density of no more than 1.05 mg/mm³, and even a density of no more than 1.00 mg/mm³, such as a density of no more than 0.95 mg/mm³, and even a density of no more than 0.90 mg/mm³, including a density of no more than 0.85 mg/mm³, and a density of no more than 0.80 mg/mm³, such as density of no more than 0.75 mg/mm³, and even a density of no more than 0.70 mg/mm³. A mini-tablet that is provided as a structural element in a capsule may have the same or an even lower density, as is further described below. In one embodiment, the compressed form will have a density of at least 0.50 mg/mm³, such as a density of at least 0.70 mg/mm³, and even a density of at least 0.85 mg/mm³. For example, a density of the compressed form may be in the range of from 0.50 mg/mm³ to 1.11 mg/mm³, such as in the range of from 0.70 mg/mm³ to 1.05 mg/mm³, and even in the range of from 0.80 mg/mm³ to 0.95 mg/mm³.

Further embodiments of the dosage form comprising the at least one drying agent for the delivery of the active agent to the intestinal site are described in more detail below.

Tablet

In one embodiment, as shown for example in FIG. 1A, the oral dosage form 100 is provided in a tablet form (and/or caplet form), having the active agent and/or drying agent as well as any other pharmaceutically acceptable excipients that are being included in the formulation provided in powder and/or particulate form that is compressed to give the final oral dosage form. The protective coating 102 is formed on the surface 103 of the oral dosage form 100. According to one embodiment, the powders and/or particulates making up the body of the oral dosage form 100 retain boundaries and/or surfaces therebetween, for example at the particulate level, even after being compressed, but the compression nonetheless provides for a relatively compact form. In one embodiment, the oral dosage form 100 can comprise a single uniform body of the compressed material, for example having the active agent, at least one drying agent, and/or other pharmaceutically acceptable agents, with the components being relatively uniformly distributed throughout the body of the tablet. In yet another embodiment, an example of which is shown in FIG. 1A, the tablet comprises first and second regions 101,105 having different compositions, such as a first layer 104 having a first composition, and a second layer 106 having a second composition that is different than the first composition. For example, the first layer 104 can comprise a drying composition including the at least one drying agent, whereas the second layer 106 can comprise a composition including the active agent, and optionally also including another component such as a permeation enhancer and/or gelling agent.

In one embodiment, by providing the separate layers 104 for the drying composition and the composition including the active agent, the drying effect can be achieved by a bulk delivery of the drying agent, with less of a dilution effect that might result by including the active agent and/or other agents such as the gelling agent in the layer 104 containing the drying agent. Furthermore, in a case where a gelling agent is provided with the active agent in the second layer 106, the gelling agent can form a gel-like mass with the active agent for delivery thereof, with reduced interference from the drying composition. In yet another case where a permeation enhancer is provided with the active agent in the second layer 106, the permeation enhancer can enhance delivery of the active agent through the intestinal tissue. That is, by separating the compositions into the two layers 104 and 106, the composition of each layer may be better able perform its intended function, without as much interference from the other layer. Additionally and/or alternatively, in one embodiment, the other component such as the permeation enhancer is provided in both the first layer 104 containing the drying agent and the second layer 106 containing the active agent, and/or in another embodiment the permeation enhancer may be provided only in the first layer 104 containing the drying agent. In yet another embodiment, the other component such as the gelling agent is provided in both the first layer 104 containing the drying agent and the second layer 106 containing the active agent, and/or in another embodiment the gelling agent may be provided only in the first layer 104 containing the drying agent.

In yet another embodiment as shown in FIG. 1B, the oral dosage form 100 is in the form of a tablet having first, second and third layers 104, 106 and 108. The first, second and third layers 104, 106 and 108 may have first, second and third compositions that are different from one another, or alternatively at least two of the layers may share the same composition, which is different than the third. The configuration of the first, second and third layers may also be selected to provide enhanced delivery of the components of the oral dosage form 100. For example, second and third layers 106 and 108 may be outer layers, while first layer 104 may be an inner layer, or vice versa. The layers may have substantially planar surfaces with one another, or the layers may form a core-shell type structure, with each layer being circumferentially interior to another layer. For example, in the embodiment as shown in FIG. 1C, the first layer 104 is a circumferentially interior layer and/or core layer, with the second layer 106 at least partially surrounding the interior layer and/or forming a shell about the interior layer. Alternatively, the first layer 104 may at least partially concentrically surround the second layer 106, and/or the tablet form can comprise a plurality of concentric layers. Other layer configurations can also be devised. For example, in one embodiment as shown in FIG. 1B, the second and third layers 106, 108 form outer layers of the tablet, and the first layer 104 is an interior layer in between the second and third layers 106, 108. According to one embodiment, the first layer 104 comprises a drying composition, whereas the second and third layers 106, 108 comprise a composition including the active agent, and optionally also including a gelling agent. The composition of the layers can also be reversed, with the second and third outer layers 106, 108 comprising the drying composition and the second interior layer comprising the active agent and optionally a gelling agent, and other configurations can also be provided.

In one embodiment, the tablet formulation is devised to provide a release of the at least one drying agent and/or active agent that imparts enhanced bioavailability of the active agent. For example, according to one aspect, the tablet can comprise a first compressed region 105 having the at least one active agent to be delivered to the intestinal site, and a second compressed region 101 having the at least one drying agent in a total drying agent percent by weight of at least 10 wt % of the second compressed region. For example, the at least one drying agent may be provided in a total percent content of the second compressed region 101 that is at least 20 wt %, and even at least 25 wt %, such as at least 30 wt %, and even at least 35 wt %. In one embodiment, the at least one drying agent may be provided in a total percent content of the second compressed region that is at least 50 wt % and even at least 75 wt %, such as at least 80 wt %, and even at least 90 wt %, including at least 95 wt %, such as at least 99 wt %. For example, in one embodiment, the at least one drying agent may be provided in a total percent content of the second compressed region that is in the range of from 50 wt % to 99 wt %, including in a range of from 80 wt % to 99 wt %. In one embodiment, the first and second compressed regions 101,105 correspond to first and second layers 104,106 of the tablet formulation.

According to one embodiment, the first and second compressed regions are compressed by a applying a pressure of no more than 18,000 psi, such as a pressure of no more than 15000 psi, and even no more than 11000 psi, such as no more than 10,000 psi and even no more than 9,000 psi. In one embodiment, the first and second compressed regions are compressed by applying a pressure of at least 5000 psi, and even at least 6600 psi, such as at least 8000 psi. For example, the first and second compressed regions may be compressed by applying at a pressure of at least 5000 psi and no more than 18,000 psi, such as a pressure of at least 6000 psi and no more than 15000, and even a pressure of at least 8000 psi and no more than 11000 psi. In a further embodiment, the first and second compressed regions may be compressed at a compression force and/or compressive pressure to provide a compressed form having a relatively lower density as measured in mg of the dosage form per volume of the dosage form. For example, the first and second compressed regions (e.g., tablet or minitablet) may have a density of no more than 1.11 mg/mm³, such as a density of no more than 1.05 mg/mm³, and even a density of no more than 1.00 mg/mm³, such as a density of no more than 0.95 mg/mm³, and even a density of no more than 0.90 mg/mm³, including a density of no more than 0.85 mg/mm³, and a density of no more than 0.80 mg/mm³, such as density of no more than 0.75 mg/mm³, and even a density of no more than 0.70 mg/mm³. In one embodiment, the first and second compressed regions will have a density of at least 0.50 mg/mm³, such as a density of at least 0.70 mg/mm³, and even a density of at least 0.85 mg/mm³. For example, a density of the first and second compressed regions may be in the range of from 0.50 mg/mm³ to 1.11 mg/mm³, such as in the range of from 0.70 mg/mm³ to 1.05 mg/mm³, and even in the range of from 0.80 mg/mm³ to 0.95 mg/mm³. According to yet another embodiment, the first and second compressed regions 101,105 may be separated by barrier layer 111, such as that described above, with the barrier layer 111 inhibiting contact between the compositions of the first and second compressed regions 101, 105. That is, the barrier layer 111 may be capable of at least partially inhibiting penetration of one or more of the first and second compressed regions 101,105 by the other compressed region, during dissolution of the dosage form in vivo. Furthermore, while first and second compressed regions 101, 105 are referred to herein, it should be understood that embodiments may further include oral dosage forms with a plurality of first and second compressed regions, such as the tablet having the multiple layers 104, 106 and 108 as shown in FIG. 1B.

According to yet another embodiment, to promote release of the drying agent and/or active agent from the oral dosage form, at least one of the first and second compressed regions can comprise a protective coating permeability promoter that promotes at least partial dissolution of the protective coating in vivo to achieve release of the contents of one or more of the first and second compressed regions. For example, in one embodiment, the protective coating permeability promoter may comprise a compound that is capable of increasing the pH in a region about the protective coating, to promote at least partial dissolution of a protective coating such as an enteric coating. The protective coating permeability promoter can comprise, for example, a basic substance provided in powder form, such as sodium bicarbonate. In one embodiment, the protective coating permeability promoter is provided substantially entirely in the compressed region 101 having that at least one drying agent, to promote release of the drying agent from the dosage form. In another embodiment, the protective coating permeability promoter is provided substantially entirely in the compressed region 105 having the at least one active agent, to promote release of the active agent from the dosage form. The protective coating permeability promoter may also be included in both the first and second compressed regions 101, 105, to promote release of the contents from the oral dosage form 100. The protective coating permeability promoter may be provided in a uniform mixture with the contents of the first and second compressed regions, and/or the protective coating permeability promoter may also, in certain embodiment, be provided as an interior coating directly beneath the protective coating 102, to act directly on the protective coating. Furthermore, by providing the protective coating permeability promoter to promote dissolution of a portion of the protective coating about one or more of the first and second compressed regions, the promoter may, in some embodiments, enhance a release rate of the contents of one or more of the first and second compressed regions from the dosage form.

According to yet another embodiment, the second compressed region 101 having the at least one drying agent may further comprise at least one binder to material to promote adhesion of the contents of the second compressed region to one another in the compressed form. For example, in one embodiment the second compressed region 101 can comprise a binder material selected from the group consisting of polyvinylpyrrolidone, HPMC and pectin, as well as mixtures thereof. According to one aspect, the binder material may be provided in a percent content of the second compressed region 101 that is at least 1 wt %, and even at least 2 wt %, such as at least 5 wt %, and even at least 8 wt %, such as at least 10 wt %. For example, the binder material may be provided in a percent content of the second compressed region that is in the range of from 1 wt % to 10 wt %, such as from 2 wt % to 8 wt %.

In one embodiment, the oral dosage form having the first and second compressed regions 105, 101 comprises at least one permeation enhancer that enhances absorption of the active agent at the intestinal site. For example, the permeation enhancer may be any of the permeation enhancers described above. The permeation enhancer may be provided in a single compressed region, or may alternatively be provided in multiple compressed regions, such as one or more of the first and second compressed regions comprising the at least one drying agent and at least one active agent. In one embodiment, the permeation enhancer is provided in the first compressed region having the at least one active agent. For example, according to one embodiment, at total content of permeation enhancer in the first compressed region may be provided in an amount of at least 5 wt %, such as at least 20 wt %, and even at least 50 wt %, such as at least 75 wt %, including at least 90 wt %, and even at least 95 wt %. For example, in one embodiment the permeation enhancer may be provided in a total content of the first compressed region of from 5 wt % to 95 wt % of the first compressed region, such as in an amount of from 20 wt % to 90 wt % of the first compressed region, and even in an amount of from 50 wt % to 90 wt % of the first compressed region. The total amount of the permeation enhancer provided in the dosage form can be selected, for example, according to the type of permeation enhancer provided, and may be any of the amounts described above that may be suitable for the dosage from. In one further embodiment, the second compressed region 101 having the at least one drying agent has little or even substantially none of the permeation enhancer included therein, for example such that the drying effect can be provided substantially without inhibiting the effects of the permeation enhancer.

Furthermore, while the tablet formulation is described above in terms of layers having different compositions, such as a first layer 104 having a drying composition and a second layer 106 having an active agent, in one embodiment the layers can also comprise the same and/or similar components, optionally in different percent contents by weight. For example, a first layer 104 having the drying composition may include not only the at least one drying agent, but also optionally a permeation enhancer and/or gelling agent. Similarly, the second layer 106 having the active agent can include another component such as the permeation enhancer and/or gelling agent, and could also include an amount of a drying agent. Accordingly, the compositions of the first, second and optionally third layers of the tablet are not limited to those specifically described therein, and tablets having multiple layers beyond the two to three layers described herein, as well as different shapes or configurations of layers in the tablets, can also be provided. Further, either as an alternative or in addition to a layered tablet, in one embodiment the tablet can comprise different sections thereof having different composition, such as an interior section having a first composition surrounded by an exterior section having a second composition. The tablet may also be a mono-layer tablet having the active agent, drying agent and/or other component such as permeation enhancer and/or gelling agent therein, either in a uniformly mixed composition or with different compositions in different regions of the mono-layer.

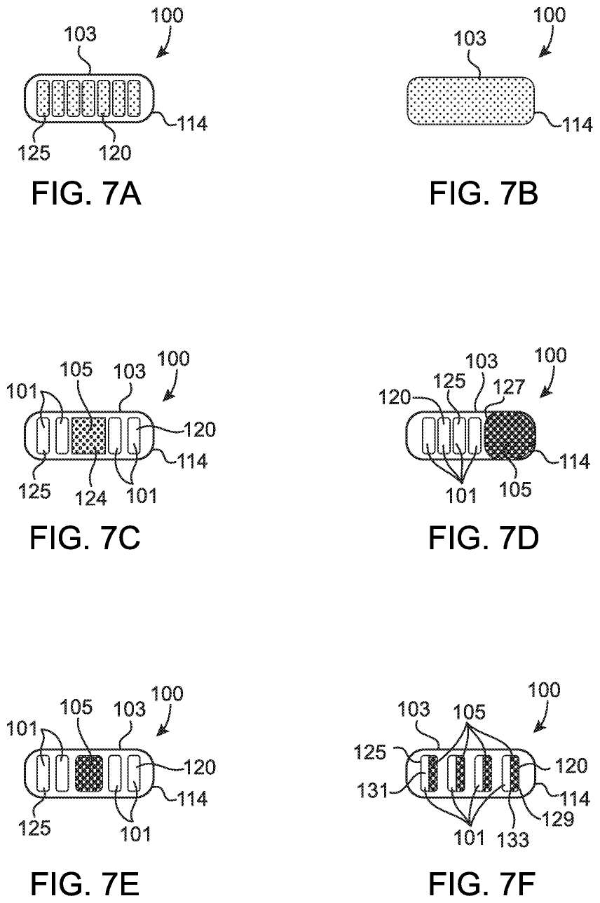

According to yet another embodiment, as shown in FIG. 7B (and an embodiment of which is also discussed in Example 1 below), a tablet is provided that has a homogeneous mixture of the active agent and drying agent therein. That is, the oral dosage form 100 can comprise a compressed tablet form that is prepared by combining the at least one active agent and at least one drying agent, such as in the amounts and contents as described in any of the tablet formulations above, and optionally with any other ingredients such as permeation enhancers, fillers and/or gelling agents, for example in any of the amounts as described above. A protective coating 103 can be provided about the capsule body 114, such as for example an enteric coating that dissolves and/or becomes permeable at a predetermined pH. The tablet formulation thus comprises a substantially homogenous mixture of active agent and drying agent, as opposed to the separate layers 104, 106 of drying agent and active agent provided in the bi-layer tablet described above (e.g., an embodiment of which is shown in FIG. 1A), and also in the bi-layer tablet with barrier layer 111 (e.g., an embodiment of which is shown in FIG. 1C). Comparing the bi-layer tablet formulation (e.g., with or without the barrier layer 111, as in FIGS. 1A and 1C) with the homogeneous tablet formulation (e.g., as in FIG. 7B), it can be understood that providing the bi-layer tablet architecture may have advantages in that the bi-layer tablets can dissolve and deliver a relatively high drug concentration in low fluid regions that are created adjacent to regions about where the drying agent from the drying agent layer 104 is released, without excessive interference between the drying agent layer 104 and the active agent layer 106 (e.g., without the drying agent layer 104 excessively absorbing amounts of active agent into a matrix of the drying agent). For example, particularly when a barrier layer 111 is provided between the drying agent layer 104 and active agent layer 106, the layers can act independently of one another to both dry and deliver active agent, without excessively inhibiting the transit of the active agent to the intestinal tissue site by the drying agent. That is, the active agent may be inhibited from getting “taken” up and absorbed into the drying agent layer 104 by the barrier layer 111, thereby freeing the active agent to contact the intestinal tissue site. However, the homogeneous tablet architecture, an embodiment of which is shown in FIG. 7B may also have advantages, in that the configuration may provide the closest possible contact between active agent and drying agent, such that the active agent is more likely to experience a relatively dry environment in its immediate vicinity upon release from the capsule. The homogeneous tablet architecture may also allow for a relatively high dose of drying agent to be provided, as the drying agent is not sequestered into a separate region. However, it should be understood that the present disclosure also encompasses embodiments and advantages that are other than those specifically described, and also that certain advantages described herein may not be provided in other embodiments.

It should also be understood that the advantages described herein with respect to the oral formulations (including capsule and tablet formulations) are provided only to further describe the embodiments, and the disclosure is not intended to be limited to any theory set forth herein, such as any theory as to whether and for which configurations certain advantages or disadvantages might arise.

Capsule

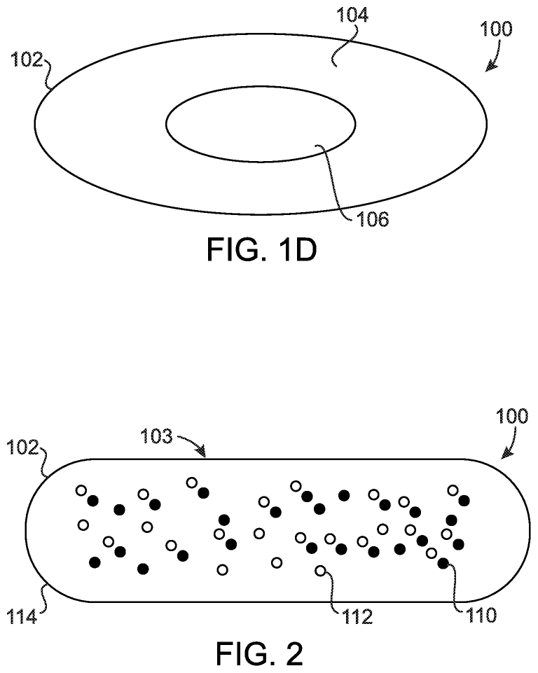

Yet another embodiment of the oral dosage form 100 is exemplified in the capsule embodiment shown in FIG. 2 and FIGS. 3A-3E. The oral dosage form 100 in capsule form according to one embodiment contains a capsule body 114 (e.g., a HPMC capsule), that is filled with materials, such particulates and/or powders, and/or other elements or phases, containing the at least one active agent and at least one drying agent, with the protective coating 102 being provided on the surface 103 of the HPMC capsule.

In one embodiment, the oral dosage form comprising the capsule has at least one active agent region 105 and at least one drying agent region 101, with one or more of the active agent region 105 and drying agent region being in the form of one or more elements 120, such as for example one or more of mini-tabs, wafers, or other structural element. According to one aspect, the active agent region 105 is provided in the form or one or more elements 120, such as one or more mini-tabs, while the drying agent region is provided in a different form, such as a powder form. According to yet another aspect, the drying agent region 101 is provided in the form of one or more elements 120, such as one or more mini-tabs, while the active agent region 105 is provided in a different form, such as a powder form or even an oil-based delivery system. According to yet another embodiment, both the active agent region 105 and drying agent region 101 are provided in the form of elements 120, and/or the dosage form may comprise both an active agent region 105 and a drying agent region 101 in the form of elements 120, optionally with additional active agent region(s) 105 and/or drying agent region(s) 101 in other forms.

According to one aspect, the elements 120 forming at least a portion of one or more of the active agent region(s) 105 and drying agent region(s) 101 are in the form of mini-tabs (i.e., mini-tablets) that are compressed at a pressure sufficient to cohere the material of the mini-tab together, while allowing good release of drying agent and/or active agent. For example, the mini-tabs may be compressed, in one embodiment, by applying a pressure of no more than 18,000 psi, such as a pressure of no more than 15000 psi, and even a pressure of no more than 12000 psi, such as a pressure of no more than 11000 psi, such as no more than 10,000 psi and even no more than 9,000 psi. In one embodiment, the mini-tabs may be compressed by applying a pressure of at least 5000 psi, and even at least 6600 psi, such as at least 8000 psi. For example, the mini-tabs may be compressed by applying at a pressure of at least 5000 psi and no more than 18,000 psi, such as a pressure of at least 6000 psi and no more than 15000, and even a pressure of at least 8000 psi and no more than 11000 psi. In a further embodiment, the mini-tabs may be compressed at a compression force and/or compressive pressure to provide a compressed form having a relatively lower density as measured in mg of the dosage form per volume of the dosage form. For example, the mini-tabs may have a density of less than 1.11 mg/mm³, such as a density of less than 1.05 mg/mm³, and even a density of less than 1.00 mg/mm³, such as a density of less than 0.95 mg/mm³, and even a density of less than 0.90 mg/mm³, including a density of less than 0.85 mg/mm³, and a density of less than 0.80 mg/mm³, such as density of less than 0.75 mg/mm³, and even a density of less than 0.70 mg/mm³. In one embodiment, the mini-tabs may have a density of at least 0.50 mg/mm³, such as a density of at least 0.70 mg/mm³, and even a density of at least 0.85 mg/mm³, such as at least 0.87 mg/mm³. For example, a density of the mini-tabs may be in the range of from 0.50 mg/mm³ to 1.11 mg/mm³, such as in the range of from 0.70 mg/mm³ to 1.05 mg/mm³, and even in the range of from 0.80 mg/mm³ to 0.95 mg/mm³.

The structural elements 120 corresponding to the mini-tabs provided in the capsule may be of substantially the same size and/or dimensions, and/or mini-tabs or structural elements of different sizes can be provided. For example, in the embodiment shown in FIG. 3A, the structural elements 120 corresponding to the mini-tabs are of substantially similar size, for both active agent mini-tabs and drying agent mini-tabs. In the embodiment shown in FIG. 3B, a structural element corresponding to a mini-tab that makes up the active agent region 105 is thicker than the structural elements 120 of the drying agent regions 101 corresponding to mini-tabs for delivery of the drying agent. That is, in one embodiment, the structural elements 120 comprising the active agent that make up the active agent region(s) 105 may be of a different size that structural elements 120 comprising drying agent that make up the drying agent region(s) 101. For example, the elements 120 comprising active agent may be larger and/or may comprise a greater volume that elements comprising the drying agent, e.g., the elements comprising the active agent may have a greater thickness along a longitudinal axis 122 than elements comprising the drying agent.

Referring to FIGS. 3A-3E, the active agent region(s) 105 and drying agent region(s) 101 can generally be understand as corresponding to those regions of the capsule where a compositions is provided, for example as a part of one or more structural elements 120 that contains either the active agent or the dying agent. For example, in the embodiment shown in FIG. 3A, the capsule comprises two active agent regions 105 corresponding to structural elements 120 in the form of mini-tabs at opposing ends of the periphery 123 of the capsule, and a central drying agent region 101 corresponding to a plurality of structural elements 120 in the form of mini-tabs at an interior 124 of the capsule. In yet another embodiment as shown in FIG. 3B, the capsule comprises one active agent region 105 located towards the interior 124 of the capsule, with two drying agent regions each comprising two structural elements 120 in the form of mini-tabs at each peripheral end of the capsule.

In one embodiment, the structural element(s) 120 comprising the at least one active agent may comprise a significant amount of the drying agent as a percent by weight of the structural element. For example, according to one aspect, a structural element, such as a mini-tablet, may comprise a total content of drying agent that is at least 50 wt % of the structural element, such as at least 75 wt % and even at least 90 wt % of a total content of drying agent as a weight percent of the structural element 120. In one embodiment, a total content of drying agent in the structural element is at least 99 wt %, such as at least 99.9 wt %. The number and composition of structural elements and/or other forms making up the drying agent regions 101 in the capsule may be such that a total content of the drying agent in the dosage form is the same as that described for the total content in the dosage form above. Furthermore, according to one aspect, a structural element, such as a mini-tablet, may comprise a total content of active agent that is at least 0.01% by weight of the structural element, such as at least 0.1 wt % and even at least 1 wt % of a total content of active agent as a weight percent of the structural element 120. For example, in one embodiment a total content of active agent in the structural element may be at least 10 wt %, such as at least 30 wt %, and even at least 50 wt %. The number and composition of structural elements and/or other forms making up the active agent regions 105 in the capsule may be such that a total content of the active agent in the dosage form is the same as that described for the total content in the dosage form above.

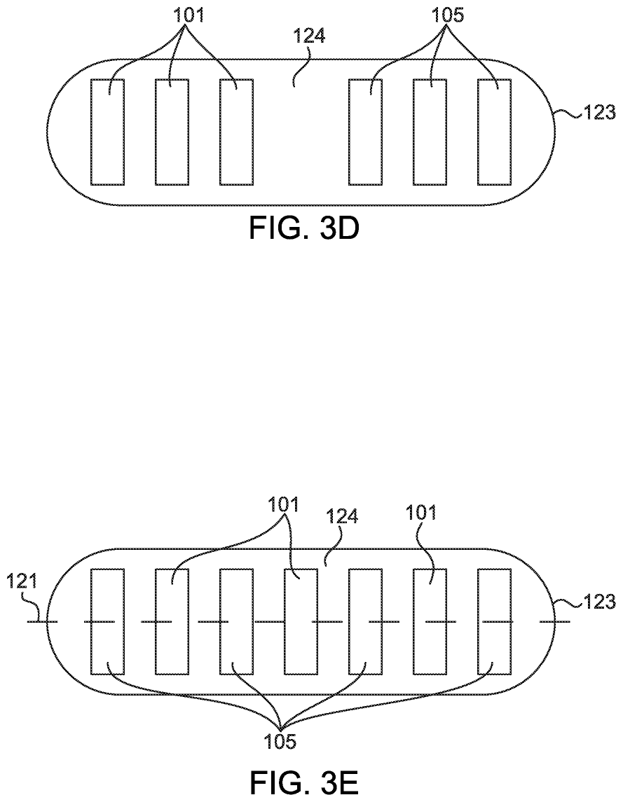

In one embodiment, the capsule comprises a plurality of structural elements 120 corresponding to one or more of the active agent and drying agent regions 105, 101. For example, the dosage form corresponding to the capsule may comprise from 1 to 15 elements (e.g., mini-tabs) therein, such as from 2 to 10 elements, and even from 3 to 6 elements. The number of structural elements provided for each of the active and drying agents may be the same, or different. For example, the number of structural elements containing drying agent may exceed the number of structural elements containing active agent, or vice versa. In the embodiments shown in FIGS. 3A-3B, the number of structural elements containing drying agent exceeds the number of structural elements containing active agent by at least 2:1 (6:1 in FIG. 3A and 4:1 in FIG. 3B). Furthermore, the configuration of the elements may be selected to provide a predetermined release of drying agent and/or active agent from the dosage form. In one embodiment, as shown in FIGS. 3A, 3C and 3E, the capsule comprises a structural element 120 having active agent at a peripheral end 123 of the capsule (i.e., at an exterior portion of the capsule, versus an interior portion), for example at just one peripheral end 123, as in FIG. 3A, or at both peripheral ends 123, as in FIGS. 3C and 3E. In yet another embodiment, as shown in FIGS. 3B and 3E, the structural element 120 comprising the active agent is provided at an interior region 124 of the capsule. Furthermore, in the versions shown in FIG. 3E, structural elements comprising the active agents are provided at both the peripheral ends 123 and at an interior region 124.

In yet embodiment, as shown in FIGS. 3B, 3C and 3D, the capsule comprises a structural element 120 having drying agent at a peripheral end 123 of the capsule, for example at just one peripheral end 123, as in FIGS. 3C and 3D, or at both peripheral ends 123, as in FIG. 3B. In yet another embodiment, as shown in FIGS. 3A, 3C and 3E, at least one structural element 120 comprising the drying agent is provided at an interior region 124 of the capsule. While not specifically, shown, structural elements 120 comprising the drying agent may also be provided at both peripheral ends 123 and at interior regions 124. Combinations of structural elements comprising the active agent and/or drying agents may also be provided in an suitable configurations, such as for example any of those shown in FIGS. 3A-3E. For example, one or more structural elements 120 comprising active agent can be provided at at least one or both peripheral ends 123, with one or more structural elements 120 comprising drying agent provided at an interior region 124. As another example, one or more structural elements 120 comprising drying agent can be provided at at least one or both peripheral ends 123, with one or more structural elements 120 comprising active agent provided at an interior region 124. Furthermore, in another embodiment, structural elements 120 comprising drying agent can be provided at one peripheral end, while structural elements 120 comprising active agent are provided at an opposing peripheral end, for example as shown in FIGS. 3C-3D. The structural elements 120 comprising active agent or drying agent can also be provided in an alternating arrangement along the longitudinal axis 121 of the capsule, as shown for example in FIG. 3E. According to yet another embodiment, a plurality of structural elements 120 comprising either the active agent or the drying agent are provided in a region of the capsule that begins at a peripheral end 123 of the capsule, and extends towards an interior region 124 of the capsule along the longitudinal axis of the capsule, as shown for example in FIGS. 3B and 3D.

Further description of the embodiments depicted in FIGS. 3B-3E is provided herein. In particular, the embodiment depicted in FIG. 3A shows a drying agent region 101 comprising 6 structural elements 120, in the form of mini-tabs, and two active agent regions 105, each comprising just a single structural element 120 in the form of a mini-tab. The active agent regions 105 comprising the mini-tab with active agent are provided at opposing first and second peripheral ends 123 of the capsule. The drying agent region 101 comprises the structural element in the form of a mini-tab that are disposed between the active agent regions in the interior region 124 of the capsule, with the mini-tabs being stacked adjacent to one another along the longitudinal axis 121 of the capsule. Without being limited to any particular theory, it is believed that providing one or more active agent regions 105 at a peripheral end 123 of the capsule may improve access of the active agent to the intestinal tissue site, to increase bioavailability.

The embodiment depicted in FIG. 3B shows two drying agent regions 101 comprising 2 structural elements 120 each, in the form of mini-tabs, and an active agent region 105 comprising just a single structural element 120 in the form of a mini-tab (the active agent mini-tab having a thickness greater than any of the individual drying agent mini-tabs). The active agent region 105 comprising the mini-tab with active agent is provided at the interior region 124 of the capsule. The drying agent regions 101 comprising structural elements in the form of a mini-tab are provided at both opposing end 123 of the capsule, with two mini-tabs on each end. Without being limited to any particular theory, it is believed that providing one or more drying agent regions 101 at a peripheral end 123 of the capsule may provide increased consistency in the ability of the drying agent to exhibit the drying effect at the intestinal site.

The embodiment depicted in FIG. 3C shows a drying agent region 101 comprising 6 structural elements 120, in the form of mini-tabs, and an active agent region 105 comprising just a single structural element 120 in the form of a mini-tab. The active agent region 105 comprising the mini-tab with active agent is provided at a first peripheral end 123 of the capsule. The drying agent region 101 comprises a structural element in the form of a mini-tab at the opposing end 123 of the capsule, with the remaining mini-tabs being sequentially aligned along the longitudinal axis and extending into the interior region 124 of the capsule. In the embodiment depicted in FIG. 3D, a drying agent region 101 comprises 3 structural elements 120, in the form of mini-tabs, and an active agent region 105 likewise comprises 3 structural elements 120, in the form of mini-tabs. Each of the drying agent region 101 and the active agent region 105 have a structural element at an opposing peripheral end 123 of the capsule, with the remaining structural elements 120 being arranged along the longitudinal axis 121 of the capsule and being lined up adjacent to one another and extending into the interior region. In the embodiment shown in FIG. 3E, an alternating arrangement of structural elements 120 corresponding to mini-tabs is provided, such that a plurality of drying agent regions 101 and active agent regions 105 are provided. In particular, in the embodiment as shown, the capsule comprises 4 active agent regions 105 each having a single structural element 120 containing the active agent therein, and comprises 3 drying agent regions 101 each having a single structural element 120 containing the drying agent therein. The structural elements 120 alternate along the longitudinal axis 121, with elements 120 containing active agent at the peripheral ends 123, and elements 120 containing drying agent disposed in between elements 120 containing the active agent.

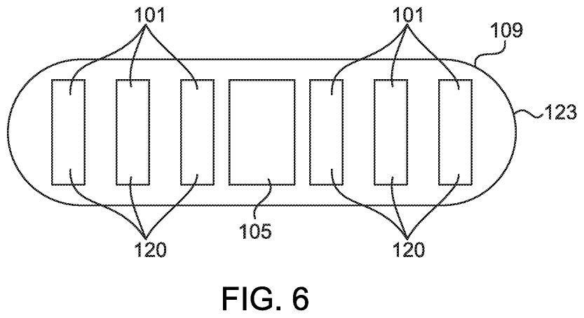

In one embodiment, the capsule formulation comprises at least one active agent region 105 that comprises a lipophilic vehicle having the at least one active agent therein. For example, the lipophilic vehicle can comprise one or more of an oil, gel, paste, semi-solid, wax, or other similar material, having the active agent dissolved or suspended therein. Without being limited to any one particular theory, it is believed that providing a lipophilic vehicle as a carrier for the at least one active agent may improve access of the active agent to intestinal tissue at the target site. In one embodiment, the lipophilic vehicle may comprise a substance that is solid at room temperature, such as a wax, but that is at least partially in liquid form at physiological temperatures. For example, the lipophilic vehicle may comprise a material that is solid at room temperature (25° C.), but that is in at least partially liquid form, or even entirely in liquid form, at physiological temperatures such as 37° C. According to one aspect, the lipophilic vehicle may be anhydrous, for example containing less than 1 wt % of water, and even less than 0.1 wt % of water, such as less than 0.01 wt % of water. Furthermore, the lipophilic vehicle containing the active agent may be provided in the capsule in a variety of different configurations. In the embodiment shown in FIG. 6, an active agent region 105 comprises an interior capsule body 107 within the exterior capsule body 109, with the interior capsule body 107 containing the lipophilic vehicle therein. In this embodiment, drying agent regions 101 containing structural elements 120 (e.g., mini-tabs) are provided at the opposing peripheral ends 23 of the capsule, with the interior capsule 107 disposed therebetween. Alternatively, embodiments may include capsules having different sections with the lipophilic vehicle therein, and/or the lipophilic vehicle may be provided at one or more the peripheral ends 23 of the capsule, with one or more drying agent regions 101 at an interior region. Furthermore, while the lipophilic vehicle containing the active agent is described above as being provided in an active agent region 105 that is separate from the drying agent region 101 (as in the embodiments shown in FIG. 6), the lipophilic vehicle may also be provided with at least some or even all of the drying agent contained therein, such as dissolved or suspended therein. Also, the lipophilic vehicle may be provided in the capsule such that it at least partially and even entirely surrounds one or more of the drying agent regions 101. For example, the lipophilic vehicle may at least partially fill the capsule such that structural elements 120 corresponding to the drying agent region(s) 101 are at least partially immersed in and/or surrounded by the lipophilic vehicle. In one embodiment, suitable materials for the lipophilic material can comprise one or more of castor oil, polyoxyalkylated sorbitol esters (such as TWEEN 80, a polyethylene sorbitol ester), mono-, di- and tri-glycerides of C₆ to C₂₂ saturated and unsaturated fatty acids, including glyceryl tricaprylate and glyceryl monocaprylate, mineral oil, a paraffin, a fatty acid, a mono-glyceride, a diglyceride, a triglyceride, an ether, and ester, olive oil, corn oil, coconut oil, peanut oil, soybean oil, cotton seed oil, sesame oil, canola oil, and combinations thereof.

Furthermore, in one embodiment, the oral dosage form comprising the capsule further contains at least one permeation enhancer that enhances absorption of the active agent at the intestinal site. For example, the permeation enhancer may be any of the permeation enhancers described above. The permeation enhancer may be provided in one or more of the active agent region 105 or drying agent region 101, or may be provided throughout the capsule, for example in a case where the capsule comprises substantially homogeneous mixture of active agent and drying agent (an embodiment of which is described in further detail below). In one embodiment, the permeation enhancer is provided in at least one active agent region 105, such as for example as contained in a structural element 120 corresponding to a mini-tab, or as contained in an active agent region 105 comprising a lipophilic vehicle. The amount of permeation enhancer providing in the active agent region(s) 105 may be a content suitable to provide a dosage suitable for the entire dosage form, and may be selected according to the particular permeation enhancer being provided, as discussed above. For example, in a case where the permeation enhancer is provided as a part of a structural element 120 such as a mini-tab, a total content of permeation enhancer in the structural element may be at least 0.1 wt %, such as at least 1 wt %, and even at least 10 wt %, such as at least 30 wt %. For example, a total content of permeation enhancer in the structural element may be in the range of from 0.1 wt % to 30 wt % of the structural element, such as in an amount of from 1 wt % to 10 wt % of the structural element. In a case where the permeation enhancer is provided as a part of a lipophilic vehicle contained in the active agent region 105, the permeation enhancer may be provided in an amount of at least 0.1 wt %, such as at least 1 wt %, and even at least 10 wt %, such as at least 30 wt %. For example, in one embodiment the permeation enhancer may be provided in the lipophilic vehicle in a content of at least 50 wt %, such as at least 70 wt %, and even at least 90 wt %. For example, the permeation enhancer may be provide in the lipophilic vehicle in a range of from 0.1 wt % to 90 wt % of the lipophilic vehicle, such as in an amount of from 1 wt % to 70 wt % of the lipophilic vehicle, and even in an amount of from 10 wt % to 50 wt % of the lipophilic vehicle. In one further embodiment, the drying agent region 101 having the at least one drying agent has little or even substantially none of the permeation enhancer included therein, for example such that the drying effect can be provided substantially without inhibiting the effects of the permeation enhancer. For example, in one embodiment, a ratio of the percent by weight of total permeation enhancer content in the active agent region 105 to the percent by weight of total permeation enhancer content in the drying agent region 101 may be at least 0.1:1, such as at least 1:1 and even at least 10:1, such as at least 30:1.

In one embodiment, the capsule contains a relatively uniform mixture of particles that includes first particles and/or powders 110 comprising the drying composition, such as the at least one drying agent, and second particles and/or powders 112 comprising the at least one active agent. Other pharmaceutically acceptable agents can also be formulated in particle form for including in the capsule, and/or can be formulated into particle form with at least one of the drying agent and active agent. In another embodiment, the first and second particles and/or powders 110 and 112 can be provided in separate regions (e.g., drying agent region(s) 101 and active agent region(s) 105) of the capsule, such as for example with first particles and/or powders 110 on one end of the capsule and second particles and/or powders 112 on the opposing end of the capsule, and/or with one of the first and second particles and/or powders 110, 112 surrounding and exterior to an internal region containing the other of the first and second particles and/or powders 110, 112. In yet another embodiment, the first and second particles and/or powders 110, 112 may be provided in a substantially uniform (i.e., homogeneous) mixture. In one embodiment, the capsule can contain the active agent and at least one drying agent blended together in a powder form. In another embodiment, at least one of the active agent and the at least one drying agent is provided in a powder form, and the other of the active agent and the at least one drying agent is provided in the form of particles. For example, the active agent can be provided in a particle form, while the drying agent is provided in a powder form, and/or the active agent can be provided in powder form while the drying agent is provided in a particle form. The particles can comprise, for example, spherically-shaped particles, such as spray-dried particle spheres, and/or can also comprise mini-tablets or mini-wafers. The particles and/or powders can comprise one or more of particles, powders, beads, grains, and combinations thereof. In one embodiment, the drying agent may be provided in a first particle size, whereas the active agent is provided in a second particle size that is different from the first particle size. For example, in one embodiment, the drying composition containing the at least one drying agent is provided in the form of a population of particles having a weight average particle size, P_{avg}, of from about 0.05 microns to about 500 microns, such as from about 5 microns to about 150 microns, and even from about 40 microns to about 60 microns. The composition having the active agent may be provided in the form of a population of particles having a weight average particle size, P_{avg}, of from about 5 microns to about 3000 microns, such as from about 150 microns to about 1000 microns, and even from about 200 microns to about 800 microns. A total content of particles comprising the at least one drying agent may be provided in a content of at least 20 wt % of the total weight of the capsule composition, such as a content of at least 40 wt %, and even at least 60 wt %, such as at least 80 wt % of the total weight of the capsule composition. For example, in one embodiment, a total content of the particles comprising the at least one drying agent may be at least 90 wt %, such as at least 95% of the total weight of the capsule composition. In one embodiment, a total content of particles comprising the at least one drying agent is provided in a range of from 20 wt % to 95 wt % of the total weight of the capsule composition, such as in a range of from 40 wt % to 90 wt %, and even in a range of from 60 wt % to 80 wt %. A total content of particles comprising the at least one active agent may be provided in a range of at least 5 wt % of the total weight of the capsule composition, such as at least 10 wt % and even at least 20 wt %, such as at least 30 wt %. For example, in one embodiment, a total content of particles comprising the at least one active agent may be at least 50 wt % and even at least 70 wt % of the total weight of the capsule composition. In one embodiment, a total content of particles comprising the at least one active agent is in a range of from 5 wt % to 70 wt % of the total weight of the capsule composition, such as in a range of from 10 wt % to 50 wt %, and even in a range of from 20 wt % to 50 wt %. The particles provided in the capsule retain boundaries and/or surface therebetween at the particulate level. Furthermore, in one embodiment the capsule can comprise a plurality of mini-wafers or mini-tablets comprising the active agent, and optionally another component such as the gelling agent, with the mini-tablets or mini-wafers being surrounded by the drying agent provided in at least one of a particulate and/or powder form. In yet another embodiment, the capsule can comprise a permeation enhancer, either blended together with one or more of the particles and/or powders, or as provided in a separate region of the capsule.

Further embodiments of capsule formulations and/or architectures are described in FIGS. 7A and 7C-7F, which architectures are also described in further detail in Example 1 below. In the capsule formulation shown in FIG. 7A, the oral dosage form 100 comprises a capsule body 114 that contains a plurality of structural elements 120 therein corresponding to mini-tablets 125. The mini-tablets 125 according to this embodiment contain a compressed composition that is a substantially homogeneous mixture of drying agent and active agent (and optionally further ingredients, such as permeation enhancer). The homogenous mixture of drying agent and active agent may provide advantages in that the dosage form 100 provides close contact between the active agent and drying agent, such that the active agent can more directly experience the drying effect provided by the drying agent. In the embodiment as shown, the dosage form comprises 6 mini-tablets having a substantially homogenous mixture of the drying agent and active agent. However, different numbers of mini-tabs can also be provided, and in varying thicknesses, and mini-tabs comprising homogeneous mixtures of drying agent and active agent can also be combined with minitabs having only drying agent or only active agent, substantially without having the other ingredient, according to aspects of the present disclosure.

In the capsule formulation shown in FIG. 7C, the oral dosage form 100 comprises a capsule body 114 that contains a plurality of structural elements 120 corresponding to mini-tablets 125 at either peripheral end 121 of the dosage form 100, and granules at an interior region 124 of the dosage form 100. In the embodiment as shown, the mini-tablets 125 comprise drying agent regions 101 comprising the at least one drying agent, and the granules correspond to an active agent region 105 comprising the at least one active agent (and optionally other ingredients, such as permeation enhancer). The dosage form as shown may provide advantages in that, in a case where permeation enhancer is provided as a part of the active agent region 105, the permeation enhancer may not significantly slow the drying agent hydration, and the active agent and optional permeation enhancer may dissolve in between multiple drying agent mini-tabs to ensure drying in the vicinity of the active agent. While the embodiment as shown depicts the granules comprising active agent at an interior region 124 of the capsule, the granules may also and/or alternatively be provided at a peripheral end 121 or other region of the capsule, or in an alternating arrangement with the mini-tabs. Furthermore, in other embodiments, the at least one drying agent may be provided in the form of granules either as an alternative to or in addition to drying agent granules, and mini-tabs comprising the at least one active agent can be provided, either alone or in combination with mini-tabs comprising the at least one drying agent.

In the capsule formulation shown in FIG. 7D, the oral dosage form 100 comprises a capsule body 114 that contains a plurality of structural elements 120 corresponding to mini-tablets 125 at a peripheral end 121 of the dosage form 100, and a lipophilic vehicle comprising the active agent at an opposing peripheral end 121 of the dosage form 100. In the embodiment as shown, the mini-tablets 125 comprise drying agent regions 101 comprising the at least one drying agent, and the lipophilic vehicle having the active agent region corresponds to an active agent region 105 comprising the at least one active agent (and optionally other ingredients, such as permeation enhancer). In one embodiment, the active agent region 105 comprising the lipophilic vehicle is bounded by a container 127 that contains the lipophilic vehicle within an area of the capsule. For example, the container 127 may comprise the capsule body 114 and a separating wall 129 interior to the capsule body 114 that separates the lipophilic vehicle from other regions of the capsule. The dosage form 100 as shown may provide advantages in that the hydrophobic lipophilic vehicle may slow uptake of the active agent into the drying agent, to provide more direct access of the active agent at the intestinal tissue site. While the embodiment as shown depicts the lipophilic vehicle comprising active agent a peripheral end 121 of the capsule, the lipophilic vehicle may also and/or alternatively be provided at the interior region 124 of the capsule, or in an alternating configuration with the mini-tabs having the at least one active agent. Furthermore, in other embodiments, the at least one drying agent may be provided in the form of granules or combination of granules and mini-tabs, and the lipophilic vehicle may also be provided without bounding to a particular region, such that the drying agent region and/or drying agent itself is immersed in and/or adjacent to the lipophilic vehicle. For example, particles of drying agent can be dispersed in the lipophilic vehicle, and/or the lipophilic vehicle may at least partially surround mini-tabs comprising the drying agent.

In the capsule formulation shown in FIG. 7E, the oral dosage form 100 comprises a capsule body 114 that contains a plurality of structural elements 120 corresponding to mini-tablets 125 throughout the capsule, with mini-tablets comprising drying agent and corresponding to drying agent regions 101 being provided at opposing ends of the capsule, and one or more mini-tabs comprising active agent (and optionally other ingredients, such as permeation enhancer) and corresponding to an active agent region(s) at the interior region 124 of the capsule. As described in Example 1 below, the active agent region 105 may further comprise an extended release material that extends and slows release of the active agent from the capsule, such as hydroxypropyl methylcellulose, and a permeation enhancer may also optionally be provided as a part of the active agent region. The extended release formulation can be provided either as a capsule formulation, as shown in FIG. 7E, or can alternatively be provided as a tablet formulation, as is described in Example 11 below. The dosage form 100 as shown may provide advantages in that the active agent (and optionally the permeation enhancer, if included in the active agent region 105), may have longer release for a longer time period for drug uptake. While the embodiment as shown depicts the drying agent regions 101 comprising mini-tabs at opposing ends 121 of the capsule, the drying agent regions may also and/or alternatively be provided at the interior region 124 of the capsule, or in an alternating configuration with the mini-tabs having the at least one active agent. Furthermore, in other embodiments, the at least one drying agent may be provided in the form of granules or combination of granules and mini-tabs. Also, while a thickness of the mini-tab having the active agent is depicted as being thicker than mini-tabs having the drying agent, the mini-tab thickness may also be the same or less, and different numbers and sizes of the mini-tabs can also be provided.

In the capsule formulation shown in FIG. 7F, the oral dosage form 100 comprises a capsule body 114 that contains a plurality of structural elements 120 corresponding to bi-layer mini-tablets 125 throughout the capsule, with the bi-layer mini-tablets an active agent layer 129 corresponding to an active agent region 105 having the at least one active agent (optionally with a permeation enhancer), and a drying agent layer 131 corresponding to a drying agent region 101 having the at least one drying agent. One or more of the mini-tablets may also optionally comprise a barrier layer 111 between each of the active and drying agent layers 129, 131. Furthermore, one or more of the mini-tablets may also be coated with its own protective layer 133, such as an enteric coating. The dosage form 100 as shown may provide advantages in provides close contact between the active agent and drying agent, similarly to a homogenous mixture, but may reduce any slowing of drying agent hydration that might occur with an intermixed permeation enhancer, in a case where permeation enhancer is provided. While the embodiment as shown depicts bi-layer tablets, it should be understood that tri-layer or other mini-table formulations can also be provide. Also, bi-layer tablets could be mixed with mono-layer tablets having only one of the drying agent or active agent. Furthermore, the tablets could be provided in a mixture with granules or active agent and/or drying agent, or a lipophilic vehicle containing one or more of active agent and drying agent.

Furthermore, the oral dosage form 100 can comprise any combination of the structural elements 120, granules, lipophilic vehicles, etc. described above, in any of the arrangements as has been described above, according to the active agent delivery that is to be provided.

Furthermore, other formulations and/or embodiments of the oral dosage formulation other than those specifically described above can also be provided. For example, a capsule formulation can also in one embodiment comprise mini-tablets containing at least one or both of the drying composition and active agent, either in addition to or as an alternative to powder and/or particular formulations. The tablet formulation can also in one embodiment comprise a tablet-in-tablet formulation, with a first interior region containing the active agent, surrounded by a second exterior region containing the drying composition, and/or a first interior region containing the drying composition, surrounded by a second exterior region containing the active agent. According to one embodiment, such a tablet-in-tablet formulation can comprise a compression coated tablet, having an interior core with a first composition corresponding to the inner tablet, and having a compression coating with a second composition surrounding the interior core to form the tablet-in-tablet structure. As yet another embodiment, at least one of the active agent and the drying composition can be provided in regions corresponding to separated spheres within a tablet.

According to one embodiment, the oral dosage form is provided in a size that provides good delivery of the active agent in the intestinal tract, without excessively occluding or blocking the intestinal tract. For example, the longest dimension of the oral dosage form may be less than about 3 cm, such as less than about 2 cm, and even less than about 1.5 cm. Typically, the longest dimension of the oral dosage form will be in the range of from about 0.5 cm to about 3 cm, such as from about 1 cm to about 3 cm, and even from about 1 cm to about 2 cm. Suitable capsule sizes may be, for example, size 1, 0, 00 and 000, and including the “EL” versions of any of these sizes.

According to one embodiment, the oral dosage form may be capable of providing a bioavailability according to an Endoscopic Bioavailability Assay of at least about 0.25%, such as a bioavailability of at least about 5% and even at least about 20%. In particular, in one embodiment, for a polypeptide having a molecular weight in a range of from about 450 Da to about 1500 Da, the oral dosage form may be capable of providing a bioavailability of at least about 2% as determined by the Endoscopic Bioavailability Assay, such as at least about 5% and even at least about 15%. In yet another embodiment, for a polypeptide having a molecular weight in the range of from about 450 Da to about 3000 Da, such as from about 500 Da to 5000 Da, the oral dosage form may be capable of providing a bioavailability of at least about 1% as determined by the Endoscopic Bioavailability Assay, such as at least about 5% and even at least about 10%. For example, in one embodiment the oral dosage form provides a bioavailability for octreotide of at least about 1% as determined by the Endoscopic Bioavailability Assay, such as at least about 5% and even at least about 10%.

Methods of Manufacturing

An oral dosage form may be manufactured by any suitable method. In certain embodiments, an oral dosage form may be manufactured under sterile conditions. In other embodiments, an oral dosage form may be sterilized prior to packaging the oral dosage form. In certain embodiments, the oral dosage form may be sterilized prior to administration to a subject. In some embodiments, an oral dosage form may be manufactured using a process including salt leaching, solvent casting, molding, spray coating, spray drying, pan coating, dip coating, waterfall coating, spin coating, and/or compression. Other methods will be known to those of ordinary skill in the art.

According to one embodiment, a tablet formulation of the oral dosage form is prepared by providing a powder of the drying composition and/or the at least one active agent, optionally also with other pharmaceutically acceptable excipients. The powder blend is loaded into a die that is pressed with a press, to form the compressed tablet body. In the case where a multiple layer tablet is prepared, powder blends corresponding to each layer may be separately loaded into the die, and sequentially pressed to form the different layers. Alternatively, other formulations for the tablet composition can also be provided as discussed above, and pressed to form the tablet composition. The tablet form can then be protectively coated on the surface of the tablet, such as enterically coated, according to embodiments of the invention. Protective coating of the tablet capsule can proceed by way of any acceptable method, such as by dip coating or spray coating of the protective coating.

According to another embodiment of the invention, a capsule formulation of the oral dosage form can be prepared by providing a powder of the drying composition and/or the at least one active agent, and filling into a pharmaceutically acceptable capsule body. Pharmaceutically acceptable capsule bodies can include, for example, hydroxypropylmethylcellulose-based capsules (HPMC capsules), gelatin capsules and/or pullulan capsules. The capsule body can be filled to provide a uniform distribution of the drying composition and/or at least one active agent, or the filling can be selectively performed to provide different concentrations of the components in different regions of the capsule, as discussed above. Furthermore, different particle sizes of the components, such as for example a different particle size of the at least one drying agent and/or drying agent composition as well as the at least one active agent, can also be provided to facilitate delivery of the active agent. The capsule may also be provided with structural elements such as mini-tabs, or oil-containing phases, as described above. Once the capsule is sealed with the composition therein, the capsule body is protectively coated on a surface thereof, such as enterically coated, according to embodiments of the invention. Protective coating of the capsule can proceed by way of any acceptable method, such as by dip coating or spray coating of the protective coating.

Other Additives for Pharmaceutical Compositions

In one embodiment, the oral dosage forms may be formulated as a unit dose. The oral dosage forms may include pharmaceutically acceptable carriers or other excipients suitable for such oral administration. Furthermore, in some embodiments, the agent may be included in the oral dosage forms in an amount sufficient to produce the desired effect upon the process or condition of the disease.

For preparing solid formulations such as tablets, the oral dosage forms may include a pharmaceutical carrier, for example, conventional tableting ingredients such as corn starch, lactose, sucrose, sorbitol, talc, stearic acid, magnesium stearate, dicalcium phosphate or gums, and optionally other pharmaceutical diluents. In solid dosage forms for oral administration (e.g., capsules, tablets, pills, dragees, powders, granules, and the like), the oral dosage forms may include one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, for example, acetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the compositions may also include buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (e.g., sodium starch glycolate or cross-linked sodium carboxymethyl cellulose), surface-active or dispersing agent. Molded tablets may be made by molding in a suitable machine a mixture of the formulation moistened with an inert liquid diluent. Tablets, and other solid dosage forms, such as dragees, capsules, pills, and granules, may optionally be scored or prepared with coatings and shells, such as enteric coatings and other coatings well known in the pharmaceutical-formulating art.

Method of Treatment

In some embodiments, an oral dosage form may be administered to an individual, patient, or a subject. In some cases, the oral dosage form may be administered as a single dosage. In other embodiments, a plurality of oral dosage forms may be administered to provide multiple dosages over time. Alternatively, the oral dosage form described herein may be administered to a subject in need thereof without food or under a fasting condition. For example, the oral dosage form may be administered at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, between about 3 hours to about 12 hours, between about 4 hours to about 12 hours, between about 4 hours to about 10 hours, between about 4 hours to about 8 hours, or between about 4 hours to about 6 hours, after consumption of food by a subject.

Alternatively, the oral dosage forms described herein may be administered to a subject in need thereof under a condition of fluid restriction. This restriction shall mean that over the stated time, the subject may consume less than 16 oz. of fluids, less than 8 oz of fluids, less than 4 oz of fluids, less than 2 oz of fluids, or less than 1 oz of fluids. For example, the subject may be restricted in their consumption of fluids prior to being administered the oral dosage form for at least about 1 hours, at least about 2 hours, at least about 3 hours, at least about 4 hours, at least about 5 hours, at least about 8 hours, between about 1 hours to about 2 hours, between about 1 hours to about 4 hours. Additionally, the subject may be restricted in their consumption of fluids after being administered the oral dosage form for at least about 1 hours, at least about 2 hours, at least about 3 hours, at least about 4 hours, at least about 5 hours, at least about 8 hours, between about 1 hours to about 2 hours, between about 1 hours to about 4 hours.

Treatment can be continued for as long or as short of a period as desired. The oral dosage form may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result is achieved. A treatment regimen can include a corrective phase, during which a dose sufficient, for example, to reduce symptoms is administered, and can be followed by a maintenance phase, during which a lower dose sufficient to maintain the reduced symptoms is administered. A suitable maintenance dose is likely to be found in the lower parts of the dose ranges provided herein, but corrective and maintenance doses can readily be established for individual subjects by those of skill in the art without undue experimentation, based on the disclosure herein.

In certain embodiments, the oral dosage form may be used to deliver an agent (e.g., octreotide) to a subject in need thereof. In some embodiments, the oral dosage form may be capable of delivering insulin to a patient in need thereof, such as a person suffering from diabetes. In certain embodiments, the oral dosage form may be used to deliver an agent (e.g., calcitonin) to a subject in need thereof. For example, the oral dosage form may be used to treat hypercalcemia. In another example, the oral dosage form may be used to treat a bone disease, such as osteoporosis. In yet another embodiment, the oral dosage form may be used to treat a mental disorder, such as bipolar disorder or mania. In yet another embodiment the oral dosage form may deliver an active agent such as a GLP-1 agonist to treat a disorder such as type II diabetes and/or obesity in a patient in need thereof. In yet another embodiment, the oral dosage form may deliver an active agent such as an enzyme-resistant peptide to treat a disorder such as a metabolic disorder to a patient in need thereof.

The oral dosage forms described herein may be used to administer an agent to patients (e.g., animals and/or humans) in need of such treatment in dosages that will provide optimal pharmaceutical efficacy. It will be appreciated that the number and/or type of oral dosage forms required for use in any particular application will vary from patient to patient, not only with the particular agent selected, but also with the concentration of agent in the oral dosage form, the nature of the condition being treated, the age and condition of the patient, concurrent medication or special diets then being followed by the patient, and other factors which those skilled in the art will recognize, with the appropriate dosage ultimately being at the discretion of the attendant physician.

Accordingly, in one embodiment, a method of delivering an active agent to a patient comprises orally administering the oral dosage form described herein, where the oral dosage form has the drying composition including the drying agent in at least 15% by weight, and the oral dosage form has the drying capacity as defined by the Agent Drying Capacity Assay for at least one of the drying agent and/or the Dosage Form Drying Capacity for the entire oral dosage formulation that meets the levels in the ranges described for the oral dosage forms above.

### EXAMPLES

### Example 1

The present example provides exemplary embodiments of oral dosage form compositions.

Bi-Layer Tablet or Capsule Formulation

TABLE 1

| Component | Function | Composition | Total wt % |
|---|---|---|---|
| Active Agent | Active agent | Octreotide | 4.7 wt % |
| Component/Drug | | | |
| Layer | | | |
| | Gelling Agent | Pectin | 4.2 wt % |
| | Osmagent | Sucrose | 37.9 wt % |
| Drying Composition | Drying Agent | Crosscarmellose | 37.4 wt % |
| Component/Drying | | (Ac-di-sol) | |
| Composition Layer | | | |
| | Binder | Pectin | 9.3% wt % |
| Protective Coating | Enteric Coating | Eudragit L-100 | 6.5 wt % |

Other ingredients for formulating in the oral dosage form, in addition to or as an alternative to those above, may be as follows:

Active agents: exenatide, salmon calcitonin, PTH (1 wt %, 10 wt %)

Gelling agents: hydroxypropylmethylcellulose (HPMC), Carbopol 934P (20 wt %, 40 wt %)

Osmagents: mannitol, fructose, PEG (8000) (10 wt %, 60 wt %) Permeation enhancers: EDTA, palmitoyl carnitine, PPS, sodium caprate (5 wt %, 20 wt %)

Drying agents: sodium polyacrylate (20 wt %, 80 wt %)

Protective coating: Eudragit S100, Kollicoat MAE 100P (3 wt %, 15 wt %)

Furthermore, the oral dosage forms as described in Table 1 above can be provided in a capsule form or tablet form. In capsule form, the composition may be in the form of powder, granules or mini-tablets, with the capsule body being formed of HPMC, gelatin and/or pullulan. The capsule size can be 1, 0, 00 or the “EL” forms of these sizes (e.g., 1EL, 0EL, or 00EL). In tablet form, the composition can form a bilayer tablet, or can form a single layer tablet, a tablet-in-tablet form (drug region surrounded by drying agent), or have spheres inside tablets. The protective coating can be formed by a spray or dip method. A protective coating that is an enteric coating can comprise a plasticizer such as triethyl citrate in an amount of 1 wt %, and/or propylene glycol in an about of 0.3 wt % or 0.1 wt %. The enteric coating can also optionally include a pore former.

Further tablet and/or capsule formulations and particular architectures therefore are described in more detail below. While specific formulations are provided, it should be noted that the formulations may also and/or alternatively comprise any of the ingredients discussed above, and may be provided in any of the capsule sizes or other specific forms as discussed above.

Additional Tablet and Capsule Formulations

TABLE 2

| | | | | Total | |
|---|---|---|---|---|---|
| Formulation | Component/ | | | amount per | |
| Architecture | Structure | Function | Composition | dosage form | |
| Barrier Bi-Layer | Active Agent | Active agent | Octreotide | 20 | mg |
| Tablet | Region | | | | |
| | Tablet layer | | | | |
| | | Enhancer | Sodium | 400 | mg |
| | | | Caprate | | |
| | | Filler | *PVP-12 | 80 | mg |
| | Drying Agent | Drying Agent | Sodium | 450 | mg |
| | Region | | Polyacrylate | | |
| | Tablet Layer | | | | |
| | | Filler | 3:1 Sodium | 50 | mg |
| | | | Bicarbonate: | | |
| | | | PVP-12 | | |
| | Barrier Layer | Barrier between | Beeswax | 100 | mg |
| | | layers | | | |
| | Protective | Enteric Coating | Eudragit L-100 | 7 | wt % |
| | Coating | | | gain | |
| Capsule with | Active Agent | Active Agent | Octreotide | 20 | mg |
| Homogeneous | Region | | | | |
| Minitablets | Minitablets | | | | |
| | | Enhancer | Sodium | 400 | mg |
| | | | Caprate | | |
| | Drying Agent | Drying Agent | Sodium | 480 | mg |
| | Region | | Polyacrylate | | |
| | Minitablets | | | | |
| | Protective | Enteric Coating | Eudragit L-100 | 7 | wt % |
| | Coating | | | gain | |
| Homogeneous | Homogeneous | Active Agent | Octeotride | 20 | mg |
| Tablet | Mixture of | | | | |
| | Active Agent | | | | |
| | and Drying | | | | |
| | Agent in Tablet | | | | |
| | Form | | | | |
| | | Enhancer | Sodium | 400 | mg |
| | | | Caprate | | |
| | | Filler | PVP-12 | 100 | mg |
| | | | 3:1 Sodium | 50 | mg |
| | | | Bicarbonate: PVP-12 | | |
| | | Drying Agent | Sodium | 675 | mg |
| | | | Polyacrylate | | |
| | Protective | Enteric Coating | Eudragit L-100 | 7 | wt % |
| | Coating | | | gain | |
| Capsule with | Active Agent | Active Agent | Octreotide | 20 | mg |
| Active Agent | Region | | | | |
| Granules/Drying | Granules | | | | |
| Agent Minitablets | | | | | |
| | | Enhancer | Sodium | 400 | mg |
| | | | Caprate | | |
| | Drying Agent | Drying Agent | Sodium | 450 | mg |
| | Region | | Polyacrylate | | |
| | Minitablets | | | | |
| | Protective | Enteric Coating | Eudragit L-100 | 7 | wt % |
| | Coating | | | gain | |
| Capsule with | Active Agent | Active Agent | Octreotide | 20 | mg |
| Active Agent in | Region | | | | |
| Oily Region | Oily Region | | | | |
| | | Enhancer | Sodium | 150 | mg |
| | | | Caprate | | |
| | | Oily Phase | *Tween 80, | 350 | μL |
| | | | castor oil, | | |
| | | | glyceryl | | |
| | | | tricaprylate | | |
| | | | and glyceryl | | |
| | | | monocaprylate | | |
| | | Filler | PVP-12 | 30 | mg |
| | Drying Agent | Drying Agent | Sodium | 450 | mg |
| | Region | | Polyacrylate | | |
| | Minitablets | | | | |
| | Protective | Enteric Coating | Eudragit | 7 | wt % |
| | Coating | | L-100 | gain | |
| Enhancer | Active Agent | Active Agent | Octreotide | 20 | mg |
| Extended | Region | | | | |
| Release (Tablet | Tablet Layer or | | | | |
| or Capsule) | Minitablet in | | | | |
| | Capsule | | | | |
| | | Enhancer | Sodium | 400 | mg |
| | | | Caprate | | |
| | | Filler | *HPMC | 20 | mg |
| | Drying Agent | Drying Agent | Sodium | 450 | mg |
| | Region | | Polyacrylate | | |
| | | Filler | 3:1 Sodium | 50 | mg |
| | | | Bicarbonate: | | |
| | | | PVP-12 | | |
| | Barrier Layer | Barrier | Beeswax | 100 | mg |
| | (in Tablet | between | | | |
| | formulation) | layers (in Tablet | | | |
| | | formulation) | | | |
| | Protective | Enteric Coating | Eudragit L-100 | 7 | wt % |
| | Coating | | | gain | |
| Capsule with | Active Agent | Active Agent | Octreotide | 20 | mg |
| Multiple Layer | Region | | | | |
| Minitablets | Layer of | | | | |
| | Minitablet | | | | |
| | | Enhancer | Sodium | 400 | mg |
| | | | Caprate | | |
| | | Filler | PVP-12 | 80 | mg |
| | Drying Agent | Drying Agent | Sodium | 360 | mg |
| | Region | | Polyacrylate | | |
| | Layer of | | | | |
| | Minitablet | | | | |
| | | Filler | 3:1 Sodium | 40 | mg |
| | | | Bicarbonate: | | |
| | | | PVP-12 | | |
| | Protective | Enteric Coating | Eudragit L-100 | 7 | wt % |
| | Coating (about | | | gain | |
| | each minitablet | | | | |
| | and about the | | | | |
| | entire capsule) | | | | |

*PVP-12 is a polyvinylpyrrolidone polymer, commercially available as BASF Kollidon-12
*Tween 80 is the trade name for Polysorbate 80, a polyethylene sorbitol ester, and is commercially available from Sigma-Aldrich.
*HPMC is a hydroxypropyl methylcellulose.

dosage formulations in Table 2 are described in more detail in Example 3 below.

Embodiments of the tablet and capsule formulations listed in Table 2 are depicted schematically in FIG. 1C (Barrier Bi-Layer Tablet), as well as FIG. 7A (Capsule with Homogeneous Minitablets), FIG. 7B (Homogeneous Tablet), FIG. 7C (Capsule with Active Agent Granules/Drying Agent Minitablets), FIG. 7D (Capsule with Active Agent in Oily Region), FIG. 7E (Enhancer Extended Release Capsule) and FIG. 7F (Capsule with Multiple Layer Minitablets). Examples of embodiments of methods of manufacturing the oral dosage forms are provided in Examples 6-12 below.

Furthermore, the different architectures for the oral dosage form, such as those listed in Table 2 above, may provide certain advantages for the delivery of active agents. Without being limited to any particular, theory of operation of the oral dosage forms herein, a number of different advantages that may be associated with each of the architectures set forth in Table 2. For example, the barrier bi-layer tablet may, in certain embodiments provide close proximity of drying agent layer and active agent layer, and may allow for delivery of active agent at high concentration in low-fluid regions adjacent the drying agent layer. As yet another example, the capsule with homogenous mini-tablets may, in certain embodiments, provide for close contact between active agent and drying agent, while also allowing for relatively quick dissolution of the mini-tablets. As yet another example, the homogeneous tablet may, in certain embodiments, allow for a relatively high drying agent content, while also providing close contact between the active agent and permeation enhancer. As yet another example, the capsule with active agent granules (in interior)/drying agent mini-tablets may, in certain embodiments, provide less inhibition of drying agent hydration by a permeation enhancer (if provided), and the active agent (and optionally permeation enhancer) may dissolve in between multiple drying agent matrices. As yet another example, the capsule with active agent in oily region may, in certain embodiments, provide a hydrophobic oily matric that reduced uptake of active agent into drying agent regions. As yet another example, the capsule with enhancer extended release may, in certain embodiments, provide a longer release time of permeation enhancer such that a longer period of time for active agent uptake can be provided. As yet a further example, the capsule with multiple layer mini-tablets may, in certain embodiments allow for close contact between active agent and drying agent, and less inhibition of drying agent hydration, allowing higher permeation enhancer dose (if provided).

### Example 2

The present example provides exemplary embodiments of drying agents for use with embodiments of the oral dosage form described herein.

TABLE 3

| | Drying Capacity (mg |
|---|---|
| Drying Agent | PBS/mg drying agent) |
| Ac-Di-Sol SD 411 NF | 7.70 |
| Sodium Polyacrylate | 19.61 |
| Ac-Di-Sol SDW 802 NF | 5.13 |
| Sodium Carboxymethylcellulose | 3.00 |
| (MW-90000) | |
| Neusilin UFL2 | 4.72 |
| Neusilin U52 | 5.85 |

As a comparison, the polymeric composition known as Avicel PH 101 NF (microcrystalline cellulose) has a value of 2.63 (mg PBS/mg Avicel).

### Example 3

The present example provides exemplary embodiments of drying agents for use with embodiments of the oral dosage form described herein. The media uptake ratios (MUR) at time points of 15, 30, 60 and 120 minutes were determined for each sample of drying agent, by performing the Agent Fluid Uptake Assay described above to obtain a fluid absorbed for each drying agent sample per mass of the sample at each of the time points. The MUR at 120 mins (2 hours) for each drying agent corresponds to the fluid uptake capacity for each of the drying agents.

TABLE 4

| | | Mass | Mass Fluid | MUR(mg fluid |
|---|---|---|---|---|
| | Timepoint | Material | Absorbed | absorbed/mg |
| Material | (min) | (mg) | (mg) | drying agent) |
| Sodium Polyacrylate | 15 | 502 | 17654 | 55.09 |
| Sodium Polyacrylate | 30 | 500 | 31069 | 62.14 |
| Sodium Polyacrylate | 60 | 498 | 33600 | 67.47 |
| Sodium Polyacrylate | 120 | 502 | 39204 | 78.10 |
| Ac-Di-Sol | 15 | 503 | 7017 | 13.95 |
| Ac-Di-Sol | 30 | 502 | 6277 | 12.50 |
| Ac-Di-Sol | 60 | 498 | 8501 | 17.07 |
| Ac-Di-Sol | 120 | 501 | 9949 | 19.86 |
| Kollidon-12 (PVP) | 15 | 499 | 625 | 1.25 |
| Kollidon-12 (PVP) | 30 | 502 | 984 | 1.96 |
| Kollidon-12 (PVP) | 60 | 503 | 1356 | 2.70 |
| Kollidon-12 (PVP) | 120 | 500 | 37030 | 5.92 |
| Sodium Starch Glycolate | 15 | 502 | 12621 | 25.14 |
| (SSG) | | | | |
| Sodium Starch Glycolate | 30 | 499 | 16377 | 32.82 |
| (SSG) | | | | |
| Sodium Starch Glycolate | 60 | 502 | 19471 | 38.79 |
| (SSG) | | | | |
| Sodium Starch Glycolate | 120 | 502 | 18360 | 36.57 |
| (SSG) | | | | |

As can be seen from Table 4 above, sodium polyacrylate exhibited the highest MUR, with a value of 78 mg/mg after exposure to fluid for 2 hours.

### Example 3

The present example provides an exemplary embodiment of a method of preparing a bilayer tablet formulation of the oral dosage formulation.

A SAP powder blend is provided including 9 parts of sucrose to 1 part pectin, and materials are ground together. The active agent can further be incorporated into the materials. An ADSP powder blend is also provided containing 8 parts of Ac-Di-Sol to 2 parts pectin.

To form a 1:1 bilayer tablet, 1 part of the SAP blend is layered with 1 part of the ADSP powder, and the composition is pressed in a hydraulic press to form a tablet. To form a 3:1 bilayer tablet, 1 part of the SAP blend is layered with 3 parts of the ADSP powder, and the composition is pressed in a hydraulic press to form a tablet.

### Example 4

The present example provides an exemplary embodiment of a method of preparing a trilayer tablet formulation of the oral dosage formulation.

A SAP powder blend is provided including 9 parts of sucrose to 1 part pectin, and materials are ground together using a mechanical shaker for 60 seconds. The active agent can further be incorporated into the materials. An ADSP powder blend is also provided containing 8 parts of Ac-Di-Sol to 2 parts pectin.

To form a 3:1 trilayer tablet, 1 part of the SAP blend is layered with 3 parts of the ADSP powder, and a final layer of 1 part of the SAP blend is provided over the ADSP layered powder. The trilayer composition is pressed in a hydraulic press to form a tablet.

To form a 1:1 trilayer tablet, 1 part of ADSP blend is layers with 2 parts of the SAP powder, and a final layer of 1 part ADSP is provided over the SAP layered powder. The trilayer composition is pressed in a hydraulic press to form a tablet.

### Example 5

The present example provides an exemplary embodiment of a method of preparing a capsule formulation of the oral dosage formulation.

A SAP powder blend containing 81.0 mg sucrose, 9.0 mg pectin and 10.0 mg octreotide as active agent is provided. Ac-di-sol is added as the drying agent in an amount of 60 wt %. A HPMC capsule is provided (size 1, 0 or 00), and the SAP/active agent powder as well as drying agent powder and deposited into the body portion of the capsule. The filled capsule is sealed, and capsule is spray coated with an enteric formulation.

### Example 6

The present example provides an exemplary embodiment of a method of preparing a barrier bi-layer tablet formulation, the composition for which is described in Table 2 of Example 1 above.

In this example, 1400 mg sodium caprate, 280 mg PVP-12 and 70 mg octreotide are mixed in a 20 mL scintillation vial using a Scologex roller mixer. Separately, 5400 mg of sodium polyacrylate, 450 mg of sodium bicarbonate, and 150 mg of PVP-12 are mixed in a 20 mL scintillation vila using a Scologex roller mixer. A 500 mg aliquot of the octreotide containing blend is weighed out, and separately a 100 mg aliquot of beeswax is measured out, and a 500 mg of the sodium polyacrylate-containing blend is measured out. The 500 mg aliquot of the octreotide-containing blend is poured into a die for hydraulic tablet pressing machine. The 100 mg aliquot of beeswax is poured on top of the octreotide blend, and finally the 500 mg aliquot of sodium polyacrylate-containing blend is poured on top of the wax. The formulation is then compressed at a pressure of 4800 lbf using a carver press. The compressed tablet formulation is then removed from the press and coated to 7 wt % with a Eudragit L-100 polymer using a Caleva Mini-Coater.

### Example 7

The present example provides an exemplary embodiment of a method of preparing a capsule formulation having homogenous mini-tablets, the composition for which is described in Table 2 of Example 1 above.

In this example, 1400 mg of sodium caprate and 70 mg of octreotide are mixed in a 20 mL scintillation vial using a Scilogex roller miller. Three roughly 500 mg aliquots of the blend are weighed out, and each aliquot is compressed at 4800 lbf in a carver press. The compressed tablets are then lightly ground with a mortar and pestle to a consistent size, and three aliquots of 420 mg of the granules are weighed out. Each aliquot of 420 mg granules is mixed with 480 mg of sodium polyacrylate in separate glass vials. Each vial is weighed out into two 150 mg aliquots and six 100 mg aliquots. The 150 mg aliquots are compressed into hemispheres at 700 lbf in a carver press. The 100 mg aliquots are compressed into discs at 700 lbf in the carver press. Each set of tablets is placed in a Type 00 EL HPMC capsule, with the hemispherical tablets on the ends of the capsules. The capsules are coated to 7 wt % with Eudragit L-100 in a Caleva Mini-Coater.

### Example 8

The present example provides an exemplary embodiment of a method of preparing a homogeneous tablet formulation having a homogeneous mixture of active agent and drying agent, the composition for which is described in Table 2 of Example 1 above.

In this example, 2362 mg of sodium polyacrylate, 1400 mg of sodium caprate, 346 mg of PVP-12, 197 mg of sodium bicarbonate and 70 mg of octreotide are mixed in a 20 mL scintillation vial on a Scilogex roller mixer. An aliquot of 1250 mg of the blend is weighed out, and the aliquot is compressed at 4800 lbf in a carver press. The tablets are coated to 7 wt % with Eudragit L-100 coating using a Caleva Mini-Coater.

### Example 9

The present example provides an exemplary embodiment of a method of preparing a capsule formulation with active agent in the form of granules, and drying agent in the form of mini-tablets, the composition for which is described in Table 2 of Example 1 above.

In this example, 1400 mg of sodium caprate and 70 mg of octreotide are mixed in a 20 mL scintillation vial using a Scilogex roller mixer. Three roughly 500 mg aliquots of the blend are weighed out, and each aliquot is compressed at 4800 lbf using a carver press. The tablets are lightly ground with a mortar and pestle to a consistent granule size. Three aliquots of 420 mg of granules are weighed out. Separately, six aliquots of 150 mg of sodium polyacrylate are weighed out, and a further six aliquots of 100 mg of sodium polyacrylate are also weighed out. The 150 mg aliquots of sodium polyacrylate are compressed into hemispherical-shaped tablets at 700 lbf using a carver press, and the 100 mg aliquots of sodium polyacrylate are compressed into disc-shaped tablets at 700 lbf using a carver press. Three Type 00 EL HPMC capsules are filled with a hemispherical tablet on one end, followed by the disc tablet. Each capsule is filled with a 420 mg aliquot of granules, and a disc-shaped tablet followed by a hemispherical tablet is placed on top of the granules, and the capsule is closed. The capsules are coated to 7 wt % with Eudragit L-100 coating using a Caleva Mini-Coater.

### Example 10

The present example provides an exemplary embodiment of a method of preparing a capsule formulation with active agent in an oily region (lipophilic region), and drying agent in the form of mini-tablets, the composition for which is described in Table 2 of Example 1 above.

In this example, a blend of 75 wt % sodium caprate, 15 wt % PVP-12 and 10 wt % octreotide are provided in a total weight of 800 mg. The blend is compressed at 4800 lbf in a carver press, and the ground with mortar and pestle. A mixture of 290 μL Tween 80, 4.37 mL of castor oil, 6.59 mL of glyceryl tricaprylate and 605 μL of glyceryl monocaprylate is formed. The active agent blend is added to the oil blend at a 200 g: 300 μL ratio, to form an active agent/oil suspension. A plunger syringe is used to place 300 μL of the active agent/oil suspension in a first end of a Type 00 HPMC capsule. The bottom cap of a smaller Type 0 HPMC capsule is placed over the active agent/oil suspension inside the larger Type 00 HPMC capsule, to encapsulate the agent/oil suspension at one end of the capsule. Sodium polyacrylate is compressed into three 100 mg disc-shaped tablets and one 150 mg hemispherical-shaped tablet at 700 lbf in a carver press. The sodium polyacrylate tablets are placed in the Type 00 HPMC capsule, with the hemispherical-shaped tablet on the end opposing the agent/oil suspension, and the capsule is closed. The capsule is coated to 7 wt % with a Eudragit L-100 coating using a Caleva Mini-Coater, and the active agent/oil suspension end is further dip-coated with Eudragit S-100 coating.

### Example 11

The present example provides an exemplary embodiment of a method of preparing a tablet formulation with enhancer extended release, the composition for which is described in Table 2 of Example 1 above.

In this example, 70 mg HPMC, 70 mg octreotide, and 1400 mg sodium caprate are mixed in a 20 mL scintillation vial using the Scilogex roller mixer. Separately, 5400 mg sodium polyacrylate, 450 mg sodium bicarbonate, and 150 mg PVP-12 are mixed in a 20 mL scintillation vial using a Scilogex roller mixer. 440 mg of the sodium caprate blend is weighed out, 100 mg of beeswax is weighed out, and 500 mg of the sodium polyacrylate blend is weighed out. The sodium caprate blend is poured into a die, followed by the beeswax, and lastly followed by the sodium polyacrylate blend. The layered composition is compressed at 4800 lbf using a carver press to form tablets. The tablets are coated to 7 wt % Eudragit L-100 with a Caleva Mini-Coater.

### Example 12

The present example provides an exemplary embodiment of a method of preparing a capsule formulation with multiple bi-layer tablets, the composition for which is described in Table 2 of Example 1 above.

In this example, 1400 mg of sodium caprate and 70 mg of octreotide are mixed in a 20 mL scintillation vial using a Scilogex roller mixer, and four 110 mg aliquots of the sodium caprate blend are weighed out. Separately, 5400 mg sodium polyacrylate, 450 mg sodium bicarbonate, and 150 mg PVP-12 are mixed in a 20 mL scintillation vial using a Scilogex roller mixer, and four 100 mg aliquots of the sodium polyacrylate blend are weighed out. The sodium caprate blend and the sodium polyacrylate blend are poured into a circular flat die, and are compressed together at 700 lbf with a carver press to form the bi-layer mini-tablets. All mini-tablets are coated to 7 wt % gain using an Eudragit L-100 coating with a Caleva Mini-Coater. Four mini-tablets are placed in a Type 00 EL HPMC capsule, and the capsule is coated to 7 wt % gain using a Eudragit L-100 based coating with a Caleva Mini-Coater.

### Example 13

The present example demonstrates % bioavailabilities for compositions corresponding to the oral dosage form according to the present disclosure, as well as comparative oral dosage forms.

To determine bioavailability, the oral dosage forms according to aspects of the present disclosure, as well as comparative oral dosage forms, are tested by models including an endoscopic model (Endoscopic Bioavailability Assay), a port model (Port Bioavailability Assay) and a surgical model (Surgical Bioavailability Assay).

Endoscopic Bioavailability Assay. In the Endoscopic Bioavailability Assay, a porcine model is used, where the oral dosage form is inserted via endoscope into the animal's mouth (after administration of anesthesia) and advanced to the region of the intestine of interest, such as the duodenum or ileum. The stomach is first investigated with the endoscope. The oral dosage form to be tested is then advanced through the animal's gastrointestinal system until the region in the intestine where the oral dosage form is to be deployed is reached. The dosage form is released at the region of the intestine, and the endoscope is retracted. Blood is collected from the animal both prior to the oral dosage form placement, as well as after oral dosage form placement, to determine the level of active agent in the blood. For example, blood may be collected at 30 and 60 minutes after oral dosage form placement, as well as 2, 3, 4, 6 and 8 hours or more after the oral dosage form has been placed. Blood samples are collected after placement of the oral dosage form, and the area under the curve (AUC) is computed. The same animal is given a subcutaneous injection at a therapeutic dose, blood samples are collected at the same time intervals, and the area under the curve (AUC) is calculated. The percent bioavailability is calculated as a dose normalized ratio of the AUC of the oral dosage form divided by the AUC for the subcutaneous injection.

Surgical Bioavailability Assay. In the Surgical Bioavailability Assay, a porcine model is also used. The animals undergo open surgery such that oral dosage forms to be tested can be placed at regions of the intestine of interest, such as the jejunum and ileum. After administration of anesthesia, a ventral midline incision is made to gain access to the animal's abdominal cavity. The jejunum and ileum are exposed, and incisions are made to manually place the oral dosage forms being tested. After oral dosage form placement is complete, the intestinal incisions are closed, and the midline incision can also be temporarily closed. Also, access can be made for blood collection via the carotid artery or jugular vein. The animal can be kept under anesthesia for up to 4 hours to allow for blood collection, such as at 5 mins, 15 mins, 30 mins, 60 mins, 90 mins, and 2 hours after oral dosage form placement. Blood samples can also be collected prior to the surgical procedure. The percent bioavailability of the active agent provided with the oral dosage form is determined on the basis of the active agent levels detected in these blood samples.

Port Bioavailability Assay. In the Port Bioavailability Assay, Yucatan Minipigs are used. A medical grade jejunal tube is placed via laparotomy into the small intestine, reaching the duodenum and even the ileum. A venous access port is provided for blood collection. To test the bioavailability resulting from dosage forms, the dosage forms are placed into the small intestine through the tube, using forceps. Optionally, the interaction of the dosage form with the intestinal environment can be observed via endoscopy through the port. The animal is placed on a fasting diet for 16 hours prior to the dosage form insertion. Blood collection to assess bioavailability includes 7 draws, with one draw taking place before dosage form insertion, and subsequent draws at 30 mins., 60 mins., 1.5 hours, 2, hours, 3, hours, 4 hours, 5 hour and 6 hours after dosage form insertion. The percent bioavailability of the active agent provided with the oral dosage form is determined on the basis of the active agent levels detected in these blood samples.

FIGS. 4-5 and 8 demonstrate the bioavailability results exhibited with the oral dosage forms according to aspects of the disclosure, as well as comparative forms.

FIG. 4 shows results for a Surgical Bioavailability Assay for the administration of octreotide and calcitonin. The squares show results for administration of neat active agent powder, while circles show results for administration of a control oral dosage form. The FIG. 4 shows that introduction of neat drug in dry powder form via surgical incision provides good bioavailability even as compared to a control oral dosage form. It is believed that the good bioavailability of the neat drug powder is at least in part due to the drying effect of the neat powder at the surgically placed site as well as the optimally low fluid levels observed at the surgically placed site. Accordingly, aspects of the disclosure seek to provide a dosage form that exhibits this drying effect in an orally administrable form.

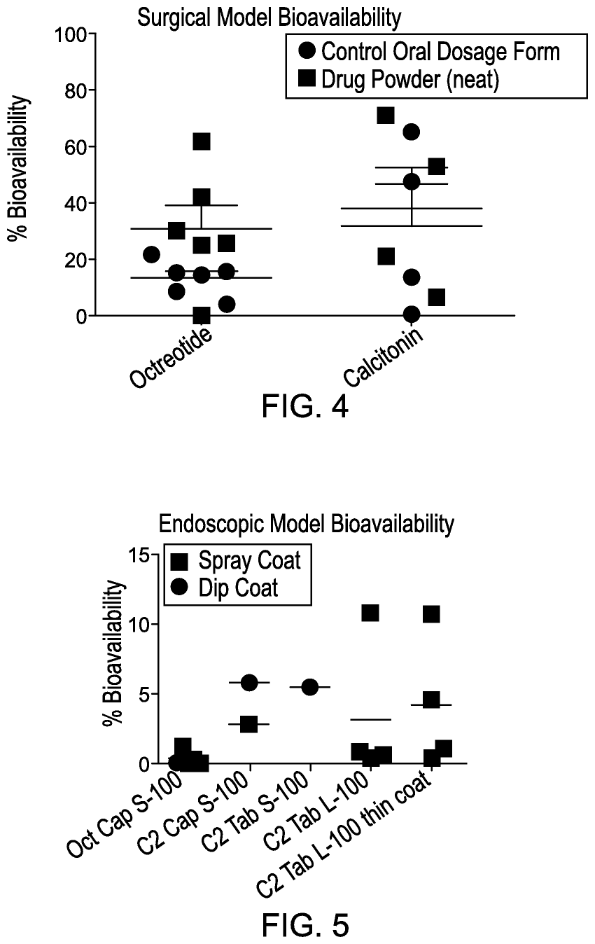

FIG. 5 shows results for an Endoscopic Bioavailability Assay using oral dosage forms according to aspects of the present disclosure, as compared to comparative oral dosage forms, with both spray and dip coats for an enteric coating. The oral dosage forms tested included a comparative oral dosage form (labelled Oct Cap S-100) having a composition of a capsule with 20 mg octreotide and a pH 7 coating (spray coated and dip coated), but without any drying agent. The oral dosage forms tested included a comparative oral dosage form (labelled C2 Cap S-100) having a composition of a capsule with 20 mg octreotide and a pH 7 coating (spray coated and dip coated), but without any drying agent. The oral dosage forms tested included an oral dosage form according to aspects of the disclosure (labelled C2 Tab S-100) having a composition of a bilayer tablet with 20 mg octreotide and a pH 7 coating (dip coated), and a composition of 180/20/200 SAP/octreotide/Ac-di-sol+pectin. The oral dosage forms tested included an oral dosage form according to aspects of the disclosure (labelled C2 Tab L-100) having a composition of a bilayer tablet with 20 mg octreotide and a pH 6 coating (spray coated), and a composition of 180/20/200 SAP/octreotide/Ac-di-sol+pectin. The oral dosage forms tested also included an oral dosage form according to aspects of the disclosure (labelled C2 Tab L-100 thin coat) having a composition of a bilayer tablet with 20 mg octreotide and a pH 7 coating (spray coated), and a composition of 180/20/200 SAP/octreotide/Ac-di-sol+pectin. The results show that good bioavailability can be achieved for oral dosage forms having a drying agent according to aspects of the present disclosure.

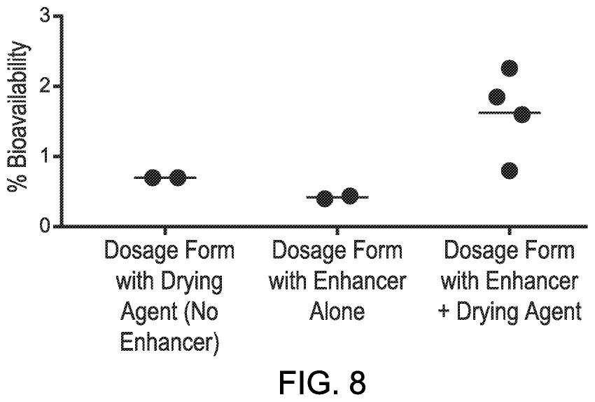

FIG. 8 shows the results for a Port Bioavailability Assay using oral dosage forms prepared according to aspects of the disclosure, and showing unexpectedly good synergistic results for the combination of permeation enhancer with drying agent in a dosage formulation. According to this example, dosage forms were prepared having either the drying agent alone (no permeation enhancer), permeation enhancer alone (no drying agent), or in a combination of both drying agent and permeation enhancer. As can be seen from FIG. 8, the combination of drying agent with permeation enhancer unexpectedly resulted in an increase in bioavailability of the active agent that was greater than the sum of the effects of either drying agent or permeation enhancer alone. That is, it can be surmised that the drying agent and permeation enhancer in combination interact in unexpected ways to improve the transport of the active agent to and/or through intestinal tissues at the target site.

In particular, the dosage form corresponding to the Dosage Form with Drying Agent (No Enhancer), the bioavailability results for which are depicted in FIG. 8 was a capsule formulation having a drying agent corresponding to sodium polyacrylate, and an active agent corresponding to octreotide, with no permeation enhancer. To prepare the dosage form, a 9:1 mixture of sucrose to pectin was prepared, and mixed with 20% octreotide in a 20 mL scintillation vial using a Scilogex roller mixer. A 100 mg aliquot of the mixture was compressed at 1300 lbf in a carver press to form a mini-tablet containing the octreotide active agent. 6 aliquots of 100 mg of sodium polyacrylate were weighed out, and each was compressed at 1300 lbf in the carver press, to form mini-tablets containing the sodium polyacrylate drying agent. The 6 sodium polyacrylate mini-tablets were inserted at one end of a size 00 capsule, and the octreotide-containing minitablet were inserted at the other end of the size 00 capsule, and the capsule was sealed. The resulting dosage form contained 20 mg of octreotide, 80 mg of the 9:1 pectin:sucrose blend, and 600 mg of sodium polyacrylate.

The dosage form corresponding to the Dosage Form with Enhancer Alone, the bioavailability results for which are depicted in FIG. 8, was a tablet formulation having an active agent corresponding to octreotide, with sodium caprate provided as the permeation enhancer. To prepare the dosage form, a 5:3:2 mixture of: (1) 9:1 sucrose:pectin; (2) sodium caprate; and (3) octreotide, was prepared, and mixed in a 20 mL scintillation vial using a Scilogex roller mixer. A 100 mg aliquot of the mixture was compressed at 1300 lbf in a carver press to form a tablet containing the octreotide active agent and permeation enhancer. The tablet was coated with an enteric coating comprising Eudragit L-100 to 7 wt % gain using a Caleva Mini-Coater. The resulting dosage form contained 20 mg of octreotide, 70 mg of the 9:1 pectin:sucrose blend, and 30 mg of sodium caprate.

The dosage form corresponding to the Dosage Form with Enhancer and Drying Agent, the bioavailability results for which are depicted in FIG. 8 was a capsule formulation having a drying agent corresponding to sodium polyacrylate, an active agent corresponding to octreotide, and a permeation enhancer corresponding to sodium caprate. To prepare the dosage form, a 5:3:2 mixture of: (1) 9:1 mixture of sucrose to pectin; (2) sodium caprate; and (3) octreotide was prepared, and mixed in a 20 mL scintillation vial using a Scilogex roller mixer. 100 mg aliquots of the mixture were compressed at 1300 lbf in a carver press to form a mini-tablet containing the octreotide active agent. 6 aliquots of 100 mg of sodium polyacrylate are weighed out, and each was compressed at 1300 lbf in the carver press, to form mini-tablets containing the sodium polyacrylate drying agent. The 6 sodium polyacrylate mini-tablets were inserted at one end of a size 00 capsule, and the octreotide and permeation enhancer-containing minitablet was inserted at the other end of the size 00 capsule, and the capsule is sealed. The capsule was coated with an enteric coating comprising Eudragit L-100 to 7 wt % gain using a Caleva Mini-Coater. The resulting dosage form contained 20 mg of octreotide, 50 mg of the 9:1 pectin:sucrose blend, 30 mg of sodium caprate, and 600 mg of sodium polyacrylate.

Accordingly, by providing a combination of the at least one drying agent with a permeation enhancer, the oral dosage form can exhibit synergistic effects in terms of the improved bioavailability of an active agent being delivered by the dosage form, over dosage forms having only drying agent or permeation enhancer alone.

Table 5 below shows the improved bioavailability results for the compositions as set forth in Table 2 of Example 1, as determined by the Port Bioavailability Assay. As can be seen from the table, differences in the architecture used can provide variations in the level of bioavailability achieved, as well as the consistency in the bioavailability levels. For example, the Capsule with Homogeneous Mini-Tablets has a greater standard deviation in the % bioavailability achieved over three trials shown as compared to the Capsule with Active Agent Granules/Drying Agent Mini-Tablets, but the Capsule with Homogeneous Mini-Tablets was also capable of achieving higher bioavailability in the trials (i.e. mean of 1.8+standard deviation of 2.0=3.8, highest data point at approximately 4%) than the trials for the Capsule with Active Agent Granules/Drying Agent Mini-Tablets (i.e., mean of 2.2+standard deviation of 0.7=2.9, highest data point at approximately 3%). Similarly, the Barrier Bi-Layer Tablet provides the highest bioavailability data points of all formulations tested (means is 2.9), and even including a trial at around 7% bioavailability. However, some inconsistency in the levels achieved is also apparent (e.g., standard deviation of 1.9), which, without being limited to any theory, may at least in part be due to orientation effects of the tablet. The Capsule with Homogenous Mini-Tablets provides a bioavailability of over 4% for at least one trial, and the Capsule with Enhancer Extended Release Tablet provides a bioavailability result for one trial of just slightly less than 4%. Good overall bioavailability levels are also seen for the other formulations. The Capsule with

Multiple-Layer Mini-Tablets provides some of the most consistent bioavailability results (standard deviation of 0.5), as do the Capsule with Active Agent Granules/Drying Agent Mini-Tablets (standard deviation of 0.7).

TABLE 5

| | Mean | | |
|---|---|---|---|
| | Bioavailability | Standard | # of |
| Architecture | (%) | Deviation | Trials |
| Barrier Bi-Layer Tablet | 2.9 | 1.9 | 9 |
| Capsule with | 1.8 | 2.0 | 3 |
| Homogeneous | | | |
| Minitablets | | | |
| Homogeneous Tablet | 1.7 | 1.0 | 3 |
| Capsule with Active | 2.2 | 0.7 | 3 |
| Agent Granules (in | | | |
| interior)/Drying Agent | | | |
| Minitablets | | | |
| Capsule with Active | 1.3 | 0.9 | 3 |
| Agent in Oily Region | | | |
| Capsule with Enhancer | 2.2 | 1.2 | 3 |
| Extended Release | | | |
| Capsule with Multiple | 1.2 | 0.5 | 3 |
| Layer Minitablets | | | |

In further embodiments, numbered 1-241 below, aspects of the present disclosure include:

### Embodiment 1

A pharmaceutically acceptable oral dosage form for delivery of an agent to an intestinal site, comprising:
one or more active agent regions comprising an active agent to be delivered to the intestinal site;
one or more drying agent regions comprising at least one drying agent therein capable of drying an area about the intestinal site, the one or more drying agent regions being separate from the one or more active agent regions; and
a protective coating at least partially covering a surface of the form,
wherein the dosage form has a fluid uptake capacity as measured for the entire dosage form when immersed in a fluid media according to a Dosage Form Fluid Uptake Assay of at least about 20 g fluid per dosage form.

### Embodiment 2

The dosage form according to Embodiment 1, wherein the fluid uptake capacity as measured for the entire dosage form when immersed in the fluid media according to the Dosage Form Fluid Uptake Assay is at least about 40 g fluid per dosage form.

### Embodiment 3

The dosage form according to Embodiment 1 or 2, wherein the fluid uptake capacity as measured for the entire dosage form when immersed in the fluid media according to the Dosage Form Fluid Uptake Assay is at least about 60 g fluid per dosage form.

### Embodiment 4

The dosage form according to any preceding Embodiment, wherein a fluid uptake time to reach the fluid uptake capacity, as determined by the Dosage Form Fluid Uptake Time Assay at pH when the dosage form is immersed in fluid media at a pH of 7.4, is no more than 2 hours.

### Embodiment 5

The dosage form according to Embodiment 4, wherein the fluid uptake time to reach the fluid uptake capacity, as determined by the Dosage Form Fluid Uptake Time Assay at pH when the dosage form is immersed in fluid media at a pH of 7.4, is no more than 30 minutes.

### Embodiment 6

The dosage form according to Embodiment 4 or 5, wherein the fluid uptake time to reach the fluid uptake capacity, as determined by the Dosage Form Fluid Uptake Time Assay at pH when the dosage form is immersed in fluid media at a pH of 7.4, is no more than 5 mins.

### Embodiment 7

The dosage form according to any preceding Embodiment, wherein a fluid uptake time to increase the total fluid uptake (MU_{D}) of the dosage form by 50%, as determined by the Dosage Form Fluid Uptake Time Assay for Uptake Phase, is no more than 2 hours.

### Embodiment 8

The dosage form according to Embodiment 7, wherein the fluid uptake time to increase the total fluid uptake (MU_{D}) of the dosage form by 50%, as determined by the Dosage Form Fluid Uptake Time Assay for Uptake Phase, is no more than 30 minutes.

### Embodiment 9

The dosage form according to Embodiment 8, wherein the fluid uptake time to increase the total fluid uptake (MU_{D}) of the dosage form by 50%, as determined by the Dosage Form Fluid Uptake Time Assay for Uptake Phase, is no more than 5 minutes.

### Embodiment 10

The dosage form according to any of the preceding Embodiments, wherein a fluid uptake time to reach the fluid uptake capacity for the dosage form, in a case where interior contents thereof are exposed to the fluid media, as determined by the Dosage Form Fluid Uptake Time Assay at Breakthrough, is no more than 2 hours.

### Embodiment 11

The dosage form according to Embodiment 10, wherein a fluid uptake time to reach the fluid uptake capacity for the dosage form, in a case where the interior contents thereof are exposed to the fluid media, as determined by the Dosage Form Fluid Uptake Time Assay at Breakthrough, is no more than 30 minutes.

### Embodiment 12

The dosage form according to Embodiment 11, wherein a fluid uptake time to reach the fluid uptake capacity for the dosage form, in a case where the interior contents thereof are exposed to the fluid media, as determined by the Dosage Form Fluid Uptake Time Assay at Breakthrough, is no more than 5 minutes.

### Embodiment 13

The dosage form according to any of the preceding Embodiments, wherein the at least one drying agent has a fluid uptake capacity when immersed in a fluid media, according to an Agent Fluid Uptake Assay, of at least about 20 (mg fluid media/mg drying agent), and wherein a Media Uptake Ratio (MUR) used to determine the fluid uptake capacity of the at least one dosage form in the Agent Fluid Uptake Assay is defined using the following formula:

MUR=(F₀−Fᵣ)/P;

where F₀−Fᵣ is the mass of fluid absorbed by the at least one drying agent in the Agent Fluid Uptake Assay, and P is the initial mass of the at least one drying agent prior to immersion in the fluid media in the Agent Fluid Uptake Assay.

### Embodiment 14

The dosage form according to Embodiment 13, wherein the at least one drying agent has a fluid uptake capacity when immersed in a fluid media, according to the Agent Fluid Uptake Assay, of at least about 40.

### Embodiment 15

The dosage form according to any of Embodiments 13-14, wherein the at least one drying agent has a fluid uptake capacity when immersed in a fluid media, according to the Agent Fluid Uptake Assay, of at least about 60.

### Embodiment 16

The dosage form according to any preceding Embodiment, wherein the at least one drying agent has a fluid uptake time to reach its fluid uptake capacity, as determined by an Agent Fluid Uptake Time Assay when the at least one drying agent is immersed in a fluid media, of no more than 30 minutes.

### Embodiment 17

The dosage form according to Embodiment 16, wherein the at least one drying agent has a fluid uptake time to reach its fluid uptake capacity, as determined by an Agent Fluid Uptake Time Assay when the at least one drying agent is immersed in a fluid media, of no more than 15 minutes.

### Embodiment 18

The dosage form according to Embodiment 16 or 17, wherein the at least one drying agent has a fluid uptake time to reach its fluid uptake capacity, as determined by an Agent Fluid Uptake Time Assay when the at least one drying agent is immersed in a fluid media, of no more than 1 minute.

### Embodiment 19

The dosage form according to any preceding Embodiment, wherein the one or more active agent regions and the one or more drying agent regions comprise discrete boundaries therebetween.

### Embodiment 20

The form according to any preceding Embodiment, wherein the one or more active agent regions comprise less than 30 wt % of the at least one drying agent, and one or more drying agent regions comprise less than 20 wt % of the at least one active agent.

### Embodiment 21

The dosage form according to any preceding Embodiment, wherein the one or more drying agent regions comprise one or more elements having the at least one drying agent therein.

### Embodiment 22

The dosage form according to any preceding Embodiment, wherein the one or more drying agent regions comprising one or more of a layer, tablet, particle, granule, bead, bulk polymeric matrix, and combinations thereof.

### Embodiment 23

The dosage form according to any preceding Embodiment, wherein the one or more active agent regions comprise one or more elements having the at least one active agent therein.

### Embodiment 24

The dosage form according to any preceding Embodiment, wherein the one or more active agent regions comprise one or more of a layer, tablet, particle, granule, bead, lipophilic vehicle, emulsion, suspension, solution, semi-solid, liquid and combinations thereof.

### Embodiment 25

The dosage form according to any of Embodiments 21-24, wherein the one or more elements each comprise a unitary structure.

### Embodiment 26

The dosage form according to any of Embodiments 21-25, wherein the dosage form is in the form of a capsule, optionally comprising the one or more elements therein.

### Embodiment 27

The dosage form according to Embodiment 26, wherein one or more of the elements are in the form of a tablet inside the capsule that is compressed by applying a pressure of at least about 5000 psi, and no more than about 18000 psi.

### Embodiment 28

The dosage form according to Embodiment 27, wherein the one or more tablets are compressed by applying a pressure of no more than about 12000 psi.

### Embodiment 29

The dosage form according to Embodiment 27 or Embodiment 28, wherein a density of the tablets (mg tablet/volume tablet) is at least about 0.7 mg/mm³, and no more than about 1.05 mg/mm³.

### Embodiment 30

The dosage form according to Embodiment 29, wherein a density of the tablets (mg drying agent/volume tablet) is no more than about 0.90 mg/mm³.

### Embodiment 31

The dosage form according to of Embodiment 1-20, wherein the dosage form is in the form of a compressed tablet having separate one or more active agent regions and one or more drying agent regions.

### Embodiment 32

The dosage form according to Embodiment 31, wherein the dosage form is compressed by applying a pressured of at least about 5000 psi, and no more than about 18000 psi

### Embodiment 33

The dosage form according to Embodiment 32, wherein the dosage form is compressed at a compression force of no more than about 10000 psi.

### Embodiment 34

The dosage form according to any of Embodiments 32-33, wherein a density of the dosage form, in mg of dosage form per volume of dosage form, is at least about 0.7 mg/mm³, and no more than about 1.05 mg/mm³.

### Embodiment 35

The dosage form according to Embodiment 34, wherein a density of the dosage form, in mg of dosage form per volume of dosage form, is no more than about 0.90 mg/mm³.

### Embodiment 36

The dosage form according to Embodiments 1-20 and 31-35, wherein the dosage form comprises a compressed tablet having separate layers corresponding to the one or more active agent regions and the one or more drying agent regions.

### Embodiment 37

The dosage form according to Embodiment 36, wherein the dosage form comprises a barrier layer between layers.

### Embodiment 38

The dosage form according to Embodiment 37, wherein the barrier layer is provided to the dosage form in a weight of in a range of from 40 mg to 400 mg per dosage form.

### Embodiment 39

The dosage form according to Embodiment 38, wherein the barrier layer is provided to the dosage form in a weight of in a range of from 50 mg to 150 mg per dosage form.

### Embodiment 40

The dosage form according to any of Embodiments 36-37, wherein the separate layers comprise one or more of upper and lower layers and concentric layers.

### Embodiment 41

The dosage form according to any of Embodiments 1-30, wherein the dosage form comprises capsule form with one or more active agent regions that comprise a lipophilic vehicle having the active agent therein.

### Embodiment 42

The dosage form according to Embodiment 41, wherein the one or more active agent regions comprise a lipophilic liquid having the at least one active agent dissolved or suspended therein.

### Embodiment 43

The dosage form according to any of Embodiments 41-42, wherein the one or more active agent regions comprise a lipophilic vehicle comprising at least one of a wax, oil, gel, semi-solid and paste.

### Embodiment 44

The dosage form according to any of Embodiments 41-43, wherein the one or more active agent regions comprise a lipophilic vehicle that is a solid at room temperature, and is at least partially in liquid form at physiological temperatures.

### Embodiment 45

The dosage form according to any of Embodiments 41-44, wherein the lipophilic vehicle having the at least one active agent therein is encapsulated in an interior capsule body.

### Embodiment 46

The dosage form according to any of Embodiments 41-45, wherein the lipophilic vehicle in the one or more active agent regions comprises less than about 2 wt % of water.

### Embodiment 47

The dosage form according to any preceding Embodiment, comprising at least one active agent region located at a peripheral end of the dosage form.

### Embodiment 48

The dosage form according to any preceding Embodiment, comprising at least one drying agent region located at a peripheral end of the dosage form.

### Embodiment 49

The dosage form according to any preceding Embodiment, comprising at least one active agent region at a first end of the dosage form, and at least one drying agent region at an opposing second end of the dosage form.

### Embodiment 50

The dosage form according to any preceding Embodiment, comprising active agent regions located at opposing peripheral ends of the dosage form, and comprising at least one drying agent region between the active agent regions.

### Embodiment 51

The dosage form according to any preceding Embodiment, comprising drying agent regions located at opposing peripheral ends of the dosage form, and comprising at least one active agent region located between the drying agent regions.

### Embodiment 52

The dosage form according to any preceding Embodiment, wherein the dosage form comprises a plurality of alternating active agent regions and drying agent regions, alternating along a longitudinal axis of the dosage form.

### Embodiment 53

The dosage form according to any preceding Embodiment, wherein the one or more active agent regions comprise a permeation enhancer.

### Embodiment 54

The dosage form according to any preceding Embodiment, comprising a permeation enhancer that is one or more of a fatty acid, medium chain glyceride, surfactant, non-steroidal detergent, acyl carnitine, lauroyl carnitine, palmitoyl carnitine, alkanoyl carnitine, N-acetylated amino acid, esters, salts, bile salts, sodium salts, nitrogen-containing rings, and derivatives and combinations thereof.

### Embodiment 55

The dosage form according to Embodiment 54, wherein the permeation enhancer is selected from the group consisting of sodium caprate, lauroyl carnitine, palmitoyl carnitine, and 3-(N,N-dimethylpalmitylammonio)propanesulfate (PPS).

### Embodiment 56

The dosage form according to any preceding Embodiment, comprising a permeation enhancer having a content of at least 5 mg per dosage form and no more than 800 mg per dosage form.

### Embodiment 57

The dosage form according to Embodiment 56, wherein the permeation enhancer is provided in a content of at least 5 mg per dosage form and no more than 50 mg per dosage form.

### Embodiment 58

The dosage form according to Embodiment 56, wherein the permeation enhancer is provided in a content of at least 50 mg per dosage form and no more than 200 mg per dosage form.

### Embodiment 59

The dosage form according to any preceding Embodiment, wherein a ratio by volume of the one or more drying agent regions to the one or more active agent regions is in a range of from 10:1 to 0.1:1.

### Embodiment 60

The dosage form according to any preceding Embodiment, wherein the form has a drying capacity as measured for the entire form according to a Dosage Form Drying Capacity Assay of at least about 20 g fluid/oral dosage form.

### Embodiment 61

The dosage form according to Embodiment 60, wherein the form has a drying capacity as measured for the entire form according to a Dosage Form Drying Capacity Assay of at least about 40 g fluid/oral dosage form.

### Embodiment 62

The dosage form according to any preceding Embodiment, wherein the at least one drying agent has a drying capacity according to an Agent Drying Capacity Assay of at least about 20 mg fluid/mg drying agent.

### Embodiment 63

The dosage form according to Embodiment 62, wherein the at least one drying agent has a drying capacity according to an Agent Drying Capacity Assay of at least about 40 mg fluid/mg drying agent.

### Embodiment 64

The dosage form according to any preceding Embodiment, wherein the at least one drying agent has a drying time as measured by an Agent Drying Time Assay of less than 1800 seconds.

### Embodiment 65

The dosage form according to Embodiment 64, wherein the at least one drying agent has a drying time as measured by an Agent Drying Time Assay of less than 900 seconds.

### Embodiment 66

The dosage form according to any preceding Embodiment, wherein the at least one drying agent comprises at least one selected from the group consisting of disintegrants, super-disintegrants, dessicants, super-absorbent polymers, swellable polymers, and super porous hydrogels and the like.

### Embodiment 67

The dosage form according to Embodiment 66, wherein the at least one drying agent comprises at least one selected from the group consisting of modified cellulose/crosslinked cellulose and derivatives thereof, croscarmellose sodium, carboxymethyl cellulose calcium, carboxymethyl cellulose sodium, hydroxypropyl cellulose, methyl cellulose, povidone, crosslinked polyvinylpyrrolidone, starch and/or modified starch, crosslinked starch, crosslinked alginic acid, sodium polyacrylate, cross-linked sodium polyacrylate, sodium starch glycolate, soy polysaccharide, gellan gum, xanthan gum, silicon dioxide, magnesium aluminum silicate, calcium silicate, and ion exchange resins.

### Embodiment 68

The dosage form according to Embodiment 67, wherein the at least one drying agent is selected from the group consisting of sodium carboxymethyl starches, cross carmellose, cross-linked sodium polyacrylate, crospovidone, and sodium starch glycolate.

### Embodiment 69

The dosage form according to any of Embodiments 1-68, wherein the at least one drying agent is comprises a polymer hydrogel having a hydrophilic polymer cross-linked with a polycarboxylic acid.

### Embodiment 70

The dosage form according to any preceding Embodiment, wherein the dosage form has a total drying agent content of at least about 10% by weight.

### Embodiment 71

The dosage form according to any preceding Embodiment, wherein the dosage form has a total drying agent content of at least about 15% by weight.

### Embodiment 72

The dosage form according to any preceding Embodiment, wherein the dosage form has a total drying agent content of at least about 30% by weight.

### Embodiment 73

The dosage form according to any preceding Embodiment, wherein the dosage form has a total drying agent content of at least about 50% by weight.

### Embodiment 74

The dosage form according to any preceding Embodiment, wherein the dosage form has a total drying agent content of at least about 75% by weight.

### Embodiment 75

The dosage form according to any preceding Embodiment, wherein the active agent comprises at least one selected from the group consisting of peptides, peptides structurally engineered to resist enzymatic degradation, antibodies, hormones, enzymes, growth factors, organic molecules, inorganic molecules, ligands, pharmaceuticals, nutraceuticals, biologics, metals, metal oxides, proteins, protein conjugates, monoclonal antibodies, polyclonal antibodies, antibody fragments, polysaccharides, carbohydrates, nanoparticles, vaccines, nucleic acids, cells and cell therapies, DNA, RNA, siRNA, blood factors, gene therapies, thrombolytic agents, growth factors, interferons, interleukin-based molecules, fusion proteins, recombinant proteins, therapeutic enzymes, drug conjugates, and metabolites.

### Embodiment 76

The dosage form according to Embodiment 75, wherein the active agent comprises at least one selected from the group consisting of octreotide, calcitonin, parathyroid hormone (PTH), teriparatide, insulin, exenatide, liraglutide, lixisenatide, albiglutide and dulaglutide.

### Embodiment 77

The dosage form according to Embodiment 75 or 76, wherein the active agent comprises a molecular weight of at least about 450 Da and less than about 10000 Da.

### Embodiment 78

The dosage form according to any of Embodiment 75-77, wherein the active agent comprise a molecular weight in the range of from about 1000 Da to about 5000 Da.

### Embodiment 79

The dosage form according to any preceding Embodiment, wherein the protective coating is capable of becoming at least partially permeable upon exposure to fluid at the intestinal site, and wherein at least 35% of the surface area of the protective coating becomes permeable upon exposure to the fluid at the intestinal site.

### Embodiment 80

The dosage form according to any preceding Embodiment, wherein a portion of the protective coating that becomes at least partially permeable upon exposure to fluid covers at least 35% of the drying agent region.

### Embodiment 81

The dosage form according to any of Embodiments 79-80, wherein substantially the entire surface of the protective coating covering the drying agent region becomes at least partially permeably upon exposure to fluid at the intestinal site.

### Embodiment 82

The dosage form according to any preceding Embodiment, wherein the protective coating comprises an enteric coating that becomes at least partially permeable and/or dissolves at a pH in the range of from 5.5 to 7.5.

### Embodiment 83

The dosage form according to any preceding Embodiment, wherein the enteric coating becomes at least partially permeable and/or dissolves at a pH of at least 5.5.

### Embodiment 84

The dosage form according to Embodiment 83, wherein the enteric coating becomes at least partially permeable and/or dissolves at a pH of at least 6.5.

### Embodiment 85

The dosage form according to Embodiment 84, wherein the enteric coating becomes at least partially permeable and/or dissolves at a pH of at least 7.4.

### Embodiment 86

The dosage form according to any preceding Embodiment, wherein the dosage form provides a release rate of the active agent of at least 90% within 30 mins, as determined by a USP Dissolution Assay 711 with Apparatus 1 and a dissolution medium of 150 mM phosphate buffered saline at a pH of 7.5.

### Embodiment 87

The dosage form according to any preceding Embodiment, wherein the dosage form provides a release rate of the active agent of at least 90% within 10 mins, as determined by a USP Dissolution Assay 711 with Apparatus 1 and a dissolution medium of 150 mM phosphate buffered saline at a pH of 7.5.

### Embodiment 88

The dosage form according to any preceding Embodiment, wherein the dosage form provides a release rate of the active agent of at least 90% within 1 minute, as determined by a USP Dissolution Assay 711 with Apparatus 1 and a dissolution medium of 150 mM phosphate buffered saline at a pH of 7.5

### Embodiment 89

A method of delivering an active agent to a subject, the method comprising:
administering the pharmaceutically acceptable dosage form of any of the preceding claims to the subject,
wherein the form provides a drying effect about the intestinal site for delivery of the active agent.

### Embodiment 90

A pharmaceutically acceptable oral dosage form for delivery of an agent to an intestinal site comprising:
an active agent to be delivered to the intestinal site;
at least one drying agent therein capable of drying an area about the intestinal site;
at least one permeation enhancer to enhance absorption of the active agent at the intestinal site; and
a protective coating at least partially covering a surface of the form,
wherein the dosage form has a fluid uptake capacity as measured for the entire dosage form when immersed in a fluid media according to a Dosage Form Fluid Uptake Assay of at least about 20 g fluid per dosage form.

### Embodiment 91

The dosage form according to Embodiment 90, wherein a total permeation enhancer content in the dosage form is in a range of from at least 5 mg per dosage form to no more than 800 mg per dosage form.

### Embodiment 92

The dosage form according to Embodiment 91, wherein the total permeation enhancer content in the dosage form is in a range of from at least 5 mg to no more than 50 mg per dosage form

### Embodiment 93

The dosage form according to Embodiment 91, wherein the total permeation enhancer content in the dosage form is at least 50 mg and no more than 200 mg per dosage form.

### Embodiment 94

The dosage form according to any of Embodiments 90-93, wherein the permeation enhancer that is one or more of a fatty acid, medium chain glyceride, surfactant, non-steroidal detergent, acyl carnitine, lauroyl carnitine, palmitoyl carnitine alkanoyl carnitine, N-acetylated amino acid, esters, salts, bile salts, sodium salts, nitrogen-containing rings, and derivatives and combinations thereof.

### Embodiment 95

The dosage form according to Embodiment 94, wherein the permeation enhancer is selected from the group consisting of sodium caprate, lauroyl carnitine, palmitoyl carnitine and 3-(N,N-dimethylpalmitylammonio)propanesulfate (PPS).

### Embodiment 96

The dosage form according to any of Embodiments 90-95, wherein the permeation enhancer is a hydrophilic permeation enhancer.

### Embodiment 97

The dosage form according to any of Embodiments 90-95, wherein the permeation enhancer is a hydrophobic permeation enhancer.

### Embodiment 98

The dosage form according to any of Embodiments 90-97, wherein the permeation enhancer has a log P of at least 2.

### Embodiment 99

The dosage form according to any of Embodiments 90-97, wherein the permeation enhancer has a log P of less than 4.

### Embodiment 100

The dosage form according to any of Embodiments 90-99, wherein the permeation enhancer comprises sodium caprate, and is provided in an amount of at least 10 mg per dosage form, and no more than 50 mg per dosage form.

### Embodiment 101

The dosage form according to Embodiment 100, wherein the sodium caprate is provided in an amount of less than 35 mg per dosage form.

### Embodiment 102

The dosage form according to any of Embodiments 90-99, wherein the permeation enhancer comprises PPS, and is provided in an amount of at least 10 mg per dosage form, and no more than 50 mg per dosage form.

### Embodiment 103

The dosage form according to Embodiment 102, wherein the PPS is provided in an amount of less than 35 mg per dosage form.

### Embodiment 104

The dosage form according to any of Embodiments 90-103, wherein the at least one permeation enhancer is one or more active agent regions of the form comprising the active agent therein, and the at least one drying agent is in one or more drying agent regions of the form, the one or more active agent regions being separate from the one or more drying agent regions.

### Embodiment 105

The dosage form according to any of Embodiments 90-104, comprising one or more active agent regions comprising less than 30 wt % of the at least one drying agent therein, and comprising one or more drying agent regions comprising less than 20 wt % of the at least one active agent therein.

### Embodiment 106

The dosage form according to any of Embodiments 90-105, further comprising an extended release agent to extend release of one or more of the active agent and permeation enhancer from the dosage form.

### Embodiment 107

The dosage form according to Embodiment 106, wherein the extended release agent comprises at least one selected from the group consisting of pectin, hydroxypropyl methyl cellulose, acrylic acid polymer and co-polymers, acacia, alginic acid, polyvinyl alcohol, sodium alginate, tragacanth, methylcellulose, poloxamers, carboxymethyl cellulose, and ethylcellulose.

### Embodiment 108

The dosage form according to any of Embodiments 90-107, comprising one or more drying agent regions comprising one or more selected from the group consisting of layers, tablets, granules, powders, beads, bulk polymeric matrices, and combinations thereof.

### Embodiment 109

The dosage form according to any of Embodiments 90-108, comprising one or more active agent regions comprising one or more selected from the group consisting of layers, tablets, granules, powders, beads, lipophilic vehicles, emulsions, suspensions, solutions, semi-solids, liquids, and combinations thereof.

### Embodiment 110

The dosage form according to any of Embodiments 90-109, wherein the dosage form comprises a capsule form having one or more drying agent regions comprising one or more tablets having the at least one drying agent therein.

### Embodiment 111

The dosage form according to any of Embodiments 90-110, wherein the dosage form comprises a capsule form comprising one or more active agent regions comprising one or more tablets having the at least one active agent and permeation enhancer therein.

### Embodiment 112

The dosage form according to any of Embodiments 90-111, wherein the dosage form comprises a capsule form comprising one or more active agent regions comprising a lipophilic vehicle having the at least one active agent and permeation enhancer therein.

### Embodiment 113

The dosage form according to any of Embodiments 90-112, wherein the dosage form comprises a capsule form having at least one of the active agent, drying agent, and permeation enhancer in the form of at least one of a powder, granule and bead.

### Embodiment 114

The dosage form according to any of Embodiments 90-113, wherein the form comprises a tablet having the at least one drying agent, at least one active agent, and at least one permeation enhancer therein.

### Embodiment 115

The dosage form according to any of Embodiments 90-114, wherein the permeation enhancer is located in region at a peripheral end of the dosage form.

### Embodiment 116

The dosage form according to any of Embodiments 90-115, wherein the permeation enhancer is located in an interior region of the dosage form.

### Embodiment 117

The dosage form according to any of Embodiments 90-116, wherein at least a portion of the at least one permeation enhancer is located at a first peripheral end of the dosage form, and at least a portion of the at least one drying agent is located at a second peripheral end of the dosage form, the first and second ends opposing one another.

### Embodiment 118

The dosage form according to any of Embodiments 90-117, wherein the permeation enhancer is located in a region in an interior of the dosage form, the interior region being in between exterior regions containing the at least one drying agent.

### Embodiment 119

The form according to any of Embodiments 90-118, wherein the permeation enhancer and at least one drying agent are provided in regions that alternate along a longitudinal axis of the dosage form.

### Embodiment 120

The dosage form according to any of Embodiments 90-119, wherein the fluid uptake capacity as measured for the entire dosage form when immersed in the fluid media according to the Dosage Form Fluid Uptake Assay is at least about 40 g fluid per dosage form.

### Embodiment 121

The dosage form according to Embodiment 120, wherein the fluid uptake capacity as measured for the entire dosage form when immersed in the fluid media according to the Dosage Form Fluid Uptake Assay is at least about 60 g fluid per dosage form.

### Embodiment 122

A method of delivering an active agent to a subject, the method comprising:
administering the pharmaceutically acceptable dosage form of any of Embodiments 90-121 to the subject,
wherein the form provides a drying effect about the intestinal site and enhances absorption of the active agent at the intestinal site.

### Embodiment 123

A pharmaceutically acceptable oral dosage form for delivery of an agent to an intestinal site comprising:
at least one active agent to be delivered to the intestinal site,
at least one drying agent provided in a percent by weight of at least 10 wt %; and
a protective coating covering a surface of the form,
wherein the dosage form is compressed at a pressure of at least 5000 psi.
wherein the dosage form has a fluid uptake capacity as measured for the entire dosage form when immersed in a fluid media according to a Dosage Form Fluid Uptake Assay of at least about 20 g fluid per dosage form.

### Embodiment 124

The dosage form according to Embodiment 123, wherein the dosage form is compressed at a pressure of no more than about 18000 psi.

### Embodiment 125

The dosage form according to Embodiment 124, wherein the dosage form is compressed at a compressive force of no more than about 10000 psi.

### Embodiment 126

The dosage form according to any of Embodiments 123-125, wherein the dosage form has a density in mg of dosage form per volume of dosage form that is in the range of from about at least about 0.7 mg/mm³ to no more than about 1.05 mg/mm³.

### Embodiment 127

The dosage form according to Embodiment 126, wherein the dosage form has a density that is no more than about 0.90 mg/mm³.

### Embodiment 128

The dosage form according to any of Embodiments 123-127, wherein the fluid uptake capacity as measured for the entire dosage form when immersed in the fluid media according to the Dosage Form Fluid Uptake Assay is at least about 40 g fluid per dosage form.

### Embodiment 129

The dosage form according to Embodiment 128, wherein the fluid uptake capacity as measured for the entire dosage form when immersed in the fluid media according to the Dosage Form Fluid Uptake Assay is at least about 60 g fluid per dosage form.

### Embodiment 130

The dosage form according to any of Embodiments 123-129, wherein the oral dosage form is in the form of at least one of a tablet and caplet.

### Embodiment 131

The dosage form according to any of Embodiments 123-130, comprising a first compressed region having the at least one active agent, and a second compressed region having the at least one drying agent.

### Embodiment 132

The dosage form according to Embodiment 131, wherein the first and second compressed regions are first and second compressed layers.

### Embodiment 133

The dosage form according to any of Embodiments 131-132, wherein the first and second compressed regions are separated by a barrier layer that inhibits contact between the first and second compressed regions.

### Embodiment 134

The dosage form according to any of Embodiments 131-133, wherein the first and second compressed regions are separated by a barrier layer that at least partially inhibits penetration of one or more of the first and second compressed region by the other compressed region during dissolution of the dosage form in vivo.

### Embodiment 135

The dosage form according to any of Embodiments 131-134, wherein the second compressed region comprises a drying agent that is at least one selected from the group consisting of modified cellulose/crosslinked cellulose and derivatives thereof, croscarmellose sodium, carboxymethyl cellulose calcium, carboxymethyl cellulose sodium, hydroxypropyl cellulose, methyl cellulose, povidone, crosslinked polyvinylpyrrolidone, starch and/or modified starch, crosslinked starch, crosslinked alginic acid, sodium polyacrylate, crosslinked sodium polyacrylate, sodium starch glycolate, soy polysaccharide, gellan gum, xanthan gum, silicon dioxide, magnesium aluminum silicate, calcium silicate, and ion exchange resins.

### Embodiment 136

The dosage form according to Embodiment 135, wherein the at least one drying agent is selected from the group consisting of sodium carboxymethyl starches, cross carmellose, crosslinked sodium polyacrylate, crospovidone, and sodium starch glycolate.

### Embodiment 137

The dosage form according to any of Embodiments 131-135, wherein the at least one drying agent is comprises a polymer hydrogel having a hydrophilic polymer cross-linked with a polycarboxylic acid.

### Embodiment 138

The dosage form according to any of Embodiments 131-135, wherein the second compressed region comprises the at least one drying agent in a content of from 10 wt % to 99 wt % of the second compressed region.

### Embodiment 139

The dosage form according to any of Embodiments 131-138, wherein the second compressed region comprises the at least one drying agent in a content of from 50 wt % to 95 wt % of the second compressed region.

### Embodiment 140

The dosage form according to any of Embodiments 131-139, wherein the second compressed region comprises less than 20% by weight of the at least one active agent, and the first compressed region comprises less than 30% by weight of the at least one drying agent.

### Embodiment 141

The dosage form according to any of Embodiments 131-140, wherein at least one of the first and second compressed regions comprise a protective coating permeability promoter that promotes at least partial dissolution of the protective coating in vivo to achieve release of contents of one or more of the first and second compressed regions.

### Embodiment 142

The dosage form according to Embodiment 141, wherein the protective coating permeability promoter comprises a compound that increases the pH about the protective coating.

### Embodiment 143

The dosage form according to any of Embodiments 141-142, wherein the protective coating permeability promoter comprises at least one base in powder form.

### Embodiment 144

The dosage form according to any of Embodiments 123-143, wherein the protective coating is an enteric coating that becomes at least partially permeable and/or at least partially dissolved at a pH in a range of from 5.5 to 7.5.

### Embodiment 145

The dosage form according to any of Embodiments 123-144, wherein the protective coating is an enteric coating that becomes at least partially permeable and/or at least partially dissolved at a pH of at least 5.5.

### Embodiment 146

The dosage form according to any of Embodiments 123-145, wherein the protective coating is an enteric coating that becomes at least partially permeable and/or at least partially dissolved at a pH of at least 6.5.

### Embodiment 147

The dosage form according to any of Embodiments 123-146, wherein the protective coating is an enteric coating that becomes at least partially permeable and/or at least partially dissolved at a pH of at least 7.4.

### Embodiment 148

The dosage form according to any of Embodiments 123-147, wherein the dosage form comprises at least one permeation enhancer that enhances absorption of the active agent at the intestinal site.

### Embodiment 149

The dosage form according to Embodiment 148, wherein the permeation wherein the permeation enhancer that is one or more of a fatty acid, medium chain glyceride, surfactant, non-steroidal detergent, acyl carnitine, lauroyl carnitine, palmitoyl carnitine, alkanoyl carnitine, N-acetylated amino acid, esters, salts, bile salts, sodium salts, nitrogen-containing rings, and derivatives and combinations thereof.

### Embodiment 150

The dosage form according to Embodiment 148 or 149, wherein the permeation enhancer is selected from the group consisting of sodium caprate, lauryl carnitine, palmitoyl carnitine, and 3-(N,N-dimethylpalmitylammonio)propanesulfate (PPS).

### Embodiment 151

The dosage form according to any of Embodiments 148-150, wherein the permeation enhancer is provided in an amount of 5 wt % to 95 wt % as a percentage of the weight of the first compressed region.

### Embodiment 152

The dosage form according to any of Embodiments 148-150, wherein the second compressed region comprises a binder material in a percent content of from 1 wt % to 10 wt % of the second compressed region.

### Embodiment 153

The dosage form according to any of Embodiments 131-152, wherein the second compressed region comprises a binder material selected from the group consisting of polyvinylpyrrolidone, HPMC, and pectin

### Embodiment 154

The dosage form according to any of Embodiments 123-130, wherein the compressed dosage form comprises a substantially uniform mixture of the at least one drying agent and the at least one active agent.

### Embodiment 155

The dosage form according to any of Embodiments 123-153, wherein the dosage form comprises a first layer comprising the at least one active agent, and a second layer comprising the at least one drying agent, and wherein a content of active agent in the second layer is less than 20 wt % by weight of the second layer, and a content of drying agent in the first layer is less than 30 wt % by weight of the first layer.

### Embodiment 156

The dosage form according to any of Embodiments 121-155, wherein the first and second layers comprise one or more of stacked layers and concentric layers.

### Embodiment 157

A pharmaceutically acceptable oral dosage form for delivery of an agent to an intestinal site comprising:
at least one active agent to be delivered to the intestinal site;
at least one drying agent capable of drying an area about the intestinal site; and
a protective coating covering a surface of the form,
wherein the at least one active agent and at least one drying agent are contained in a capsule body having the protective coating on the surface thereof, and
wherein the dosage form has a fluid uptake capacity as measured for the entire dosage form when immersed in a fluid media according to a Dosage Form Fluid Uptake Assay of at least about 20 g fluid per dosage form.

### Embodiment 158

The dosage form according to Embodiment 157, wherein one or more of the at least one active agent and the at least one drying agent are contained in a compressed element that is compressed by applying a pressure of at least 5000 psi and no more than 18000 psi.

### Embodiment 159

The dosage form according to Embodiment 158, wherein the compressed element is compressed by applying a pressure of no more than 12000 psi.

### Embodiment 160

The dosage form according to Embodiment 158 or 159, wherein a density of the compressed element in mg of compressed element per volume of compressed element is in the range of from about 0.7 mg/mm³ to about 1.05 mg/mm³.

### Embodiment 161

The dosage form according to Embodiment 160, wherein the density of the compressed element is no more than about 0.90 mg/mm³.

### Embodiment 162

The dosage form according to any one of Embodiments 151 to 156, wherein the dosage form comprises one or more active agent regions having the at least one active agent, and one or more drying agent regions having the least one drying agent.

### Embodiment 163

The dosage form according to Embodiment 157, wherein the at least one drying agent is provided in a content of at least 20% by weight of the drying agent region.

### Embodiment 164

The dosage form according to Embodiment 163, wherein the at least one drying agent is provided in a content of at least 50% by weight of the drying agent region.

### Embodiment 166

The dosage form according to Embodiment 165, wherein the at least one drying agent is provided in a content of at least 90% by weight of the drying agent region.

### Embodiment 166

The dosage form according to any of Embodiments 157-166, wherein the one or more drying agent regions comprise one or more compressed elements containing the drying agent therein.

### Embodiment 167

The dosage form according to any of Embodiments 157-166, wherein the one or more active agent regions comprise one or more compressed elements containing the active agent therein.

### Embodiment 168

The dosage form according to any of Embodiments 157-167, wherein the one or more active agent regions comprise at least one of a lipophilic vehicle, emulsion, solution, semi-solid, powder, grains and beads.

### Embodiment 169

The dosage form according to any of Embodiments 157-168, wherein the one or more active agent regions comprise a lipophilic vehicle having the active agent therein.

### Embodiment 170

The form according to any of Embodiments 157-169, wherein the one or more drying agent regions are separate from the one or more active agent regions, and wherein the one or more drying agent regions comprise less than 20 wt % of active agent and the one or more active agent regions comprise less than 30 wt % of drying agent.

### Embodiment 171

The dosage form according to any of Embodiments 157-170, wherein the form comprises a capsule body having from 2 to 10 compressed elements therein.

### Embodiment 172

The dosage form according to Embodiment 171, wherein the form comprises a capsule body having from 3 to 6 compressed elements therein.

### Embodiment 173

The dosage form according to any of Embodiments 157-172, wherein the form comprises at least one compressed element having the at least one active agent at an interior portion of the form, and comprises at least one compressed element having the at least one drying agent at an exterior portion of the form.

### Embodiment 173

The dosage form according to any of Embodiments 157-172, wherein the form comprises at least one compressed element having the at least one drying agent at an interior portion of the form, and at least one compressed element having the at least one active agent at an exterior portion of the form.

### Embodiment 174

The dosage form according to any of Embodiments 157-173, wherein compressed elements having the at least one drying agent, and compressed elements having at least one active agent are provided in an alternating arrangement along an axis of the form.

### Embodiment 175

The dosage form according to any of Embodiments 157-174, wherein the at least one active agent region comprises a permeation enhancer capable of increasing absorption of the active agent at the intestinal site.

### Embodiment 176

The dosage form according to any of Embodiments 157-175, wherein the dosage form comprises at least one compressed element having the at least one active agent and at least one permeation enhancer.

### Embodiment 177

The dosage form according to any of Embodiments 157-176, comprising one or more compressed elements having the at least one active agent and at least one permeation enhancer therein, wherein the permeation enhancer comprises at least 80 wt % of at least one compressed element.

### Embodiment 178

The dosage form according to any of Embodiments 157-177, wherein the fluid uptake capacity as measured for the entire dosage form when immersed in the fluid media according to the Dosage Form Fluid Uptake Assay is at least about 40 g fluid per dosage form.

### Embodiment 179

The dosage form according to Embodiment 178, wherein the fluid uptake capacity as measured for the entire dosage form when immersed in the fluid media according to the Dosage Form Fluid Uptake Assay is at least about 60 g fluid per dosage form.

### Embodiment 180

The dosage form according to any of Embodiments 157-179, wherein the dosage form comprises a plurality of compressed elements, each having a substantially uniform mixture of the at least one active agent and the at least one drying agent.

### Embodiment 181

The dosage form according to any of Embodiments 157-180, wherein the dosage form comprises a plurality of compressed elements, and at least one of granules, beads and powder.

### Embodiment 182

The dosage form according to Embodiment 181, wherein the plurality of compressed elements comprise the at least one drying agent, and the at least one of granules, beads and powder comprises the at least one active agent.

### Embodiment 182

The dosage form according to Embodiment 181 or 182, wherein the plurality of compressed elements are at opposing ends of the dosage form, with the at least one of granules, beads and powders in an interior region of the dosage form.

### Embodiment 183

The dosage form according to any of Embodiments 157-182, wherein the dosage form comprises a plurality of compressed elements and a lipophilic vehicle.

### Embodiment 183

The dosage form according to Embodiment 183, wherein the plurality of compressed elements comprise the at least one drying agent, and lipophilic vehicle comprises the at least one active agent.

### Embodiment 184

The dosage form according to Embodiment 183, wherein the plurality of compressed elements are at a first end of the dosage form, and the lipophilic vehicle is at a second opposing end of the dosage form.

### Embodiment 185

The dosage form according to any of Embodiments 157-184, wherein the dosage form comprises a plurality of first compressed elements comprising the at least one drying agent, and at least one second compressed element comprising the at least one active agent.

### Embodiment 186

The dosage form according to Embodiment 185, wherein the plurality of first compressed elements are at opposing ends of the dosage form, and the at least one second compressed element is at an interior region of the dosage form.

### Embodiment 187

The dosage form according to Embodiment 185 or 186, wherein the at least one second compressed element comprises a permeation enhancer and at least one release extending agent to extend release of one or more of the at least one active agent and permeation enhancer from the dosage form.

### Embodiment 188

The dosage form according to Embodiments 186-187, wherein the at least one second compressed element has a greater thickness than any one of the plurality of first compressed elements.

### Embodiment 189

The dosage form according to any of Embodiments 157-188, wherein the dosage form comprises a plurality of compressed elements, at least one of the compressed elements having at least two layers.

### Embodiment 190

The dosage form according to Embodiment 189, wherein the plurality of compressed elements comprise at least one compressed element having a first layer comprising the at least one drying agent, and a second layer comprising the at least one active agent.

### Embodiment 191

The dosage form according to Embodiment 189 or 190, wherein one or more of the plurality of compressed elements comprises a second protective coating on a surface of the compressed element.

### Embodiment 192

The dosage form according to any of Embodiments 157-159, wherein dosage form comprises first particles comprising the at least one active agent, and second particles comprising the at least one drying agent.

### Embodiment 193

The dosage form according to Embodiment 192, wherein the particles comprise at least one of powders, beads, granules or combinations thereof.

### Embodiment 194

The dosage form according to Embodiment 192 or 193, wherein the dosage form comprises a substantially homogenous mixture of the first and second particles.

### Embodiment 195

The dosage form according to any of Embodiments 193-194, comprising from 5 wt % to 70 wt % of the first particles, and from 20 wt % to 95 wt % of the second particles.

### Embodiment 196

The dosage form according any of Embodiments 157-195, wherein the dosage form comprises particles comprising the at least one drying agent having an average particle size of in the range of from about 100 microns to about 400 microns

### Embodiment 197

A pharmaceutically acceptable oral dosage form for delivery of an agent to an intestinal site comprising:
an active agent to be delivered to the intestinal site;
at least one drying agent capable of drying an area about the intestinal site; and
a protective coating covering a surface of the form,
wherein the dosage form has a total drying agent content of at least about 15% by weight.

### Embodiment 198

The dosage form according to Embodiment 197, wherein the dosage form has a total drying agent content of at least about 30% by weight.

### Embodiment 199

The dosage form according to Embodiment 198, wherein the dosage form has a total drying agent content of at least about 50% by weight.

### Embodiment 200

The dosage form according to Embodiment 199, wherein the dosage form has a total drying agent content of at least about 75% by weight.

### Embodiment 201

The dosage form according to any of Embodiments 198-200, wherein the drying agent has a drying capacity according to an Agent Drying Capacity Assay of at least about 3 mg fluid/mg drying agent.

### Embodiment 202

The dosage form according to Embodiment 201, wherein the drying agent has a drying capacity according to an Agent Drying Capacity Assay of at least about 5 mg fluid/mg drying agent.

### Embodiment 203

The dosage form according to Embodiment 202, wherein the drying agent has a drying capacity according to an Agent Drying Capacity Assay of at least about 7 mg fluid/mg drying agent.

### Embodiment 204

The dosage form according to any of Embodiments 198-203, wherein the drying agent has a drying time according to an Agent Drying Time Assay of no more than about 15 minutes.

### Embodiment 205

The dosage form according to Embodiment 205, wherein the drying agent has a drying time according to an Agent Drying Time Assay of no more than about 5 minutes.

### Embodiment 206

The dosage form according to Embodiment 205, wherein the drying agent has a drying time according to an Agent Drying Time Assay of no more than about 60 seconds.

### Embodiment 207

The dosage form according to any of Embodiments 197-206, wherein the form comprises a drying composition having the at least one drying agent, and wherein a drying capacity of the drying composition according to an Agent Drying Capacity Assay is at least about 3 mg fluid/mg drying composition.

### Embodiment 208

The dosage form according to Embodiment 207, wherein the form has a drying capacity as measured for the entire form according to a

Dosage Form Drying Capacity Assay of at least about 3 g fluid/oral dosage form.

### Embodiment 209

The dosage form according to any of Embodiments 197-208, wherein the drying agent has a solubility in water such that a viscosity of a liquid part of a solution of water containing 5 mg of the drying agent/mL water at standard temperature and pressure is less than 5 cP.

### Embodiment 210

The dosage form according to any of Embodiments 196-209, wherein the at least one drying agent comprises at least one of cross carmellose, sodium polyacrylate, crospovidone, and sodium starch glycolate.

### Embodiment 211

The dosage form according to any of Embodiments 196-210, further comprising a gelling agent capable of forming a gel upon exposure to an intestinal environment.

### Embodiment 212

The dosage form according to Embodiment 211, wherein the gelling agent comprises at least one of pectin, hydroxypropylmethylcellulose and an acrylic acid polymer/copolymer.

### Embodiment 213

The dosage form according to Embodiment 212, wherein a content of the gelling agent in the form is from about 1 wt % to about 50 wt %.

### Embodiment 214

The dosage form according to any of Embodiments 197-213, wherein the drying agent is provided in a first region of the form, and the active agent is provided in second region of the form.

### Embodiment 215

The dosage form according to Embodiment 214, wherein the first and second regions comprise first and second layers of a bi-layer tablet.

### Embodiment 216

The dosage form according to Embodiment 215, wherein the first and second regions comprise first and second layers of a tri-layer tablet, the tri-layer tablet further comprising a third layer having a third composition that is the same and/or different from one or more of the first and second layers.

### Embodiment 217

The dosage form according to Embodiment 214, wherein the first and second regions are regions of a compression coated tablet having a core and a compression coating at least partially surrounding the core.

### Embodiment 218

The dosage form according to Embodiment 217, wherein the core comprises the first region having the drying agent, and the compression coating comprises the second region having the active agent.

### Embodiment 219

The dosage form according to Embodiment 218, wherein the core comprises the second region having the active agent, and the compression coating comprises the first region having the drying agent.

### Embodiment 220

The dosage form according to Embodiment 214, wherein a gelling agent is contained in at least one of the first region containing the at least one drying agent and the second region containing the active agent.

### Embodiment 221

The dosage form according to any of Embodiments 197-220, wherein the form comprises a mono-layer tablet.

### Embodiment 222

The dosage form according to any of Embodiments 197-221, wherein at least one of the drying agent and the active agent is provided in a particulate form.

### Embodiment 223

The dosage form according to any of Embodiments 197-222, wherein the form comprises a capsule form.

### Embodiment 224

The dosage form according to Embodiment 223, wherein at least one of the active agent and the least one drying agent are provided in a powdered form in the capsule.

### Embodiment 225

The dosage form according to Embodiment 224, wherein at least one of the active agent and the at least one drying agent are provided in the form of particles in the capsule

### Embodiment 226

The dosage form according to Embodiment 225, wherein the particles comprise at least one of spheres and tablets.

### Embodiment 227

The dosage form according to Embodiment 226, wherein the capsule comprises a plurality of tablets having the active agent, and wherein the drying agent at least partially surrounds the tablets.

### Embodiment 228

The dosage form according to any of Embodiments 197-227, wherein the protective coating comprises at least one of a pH-dependent enteric coating and a timed-release coating.

### Embodiment 229

The dosage form according to Embodiment 228, wherein the protective coating is an enteric coating that is capable of releasing the active agent from the form at a pH of from about 5.5 to about 7.5.

### Embodiment 230

The dosage form according to Embodiment 229, wherein the enteric coating comprises at least one of poly(methacrylic acid-co-methyl methacrylate) and methacrylic acid ethyl acrylate copolymer.

### Embodiment 231

The dosage form according to any of claims 197-230, wherein the active agent comprises at least one of octreotide, calcitonin, parathyroid hormone, teriparatide, insulin, exenatide, liraglutide, lixisenatide, albiglutide and dulaglutide.

### Embodiment 232

The dosage form according to any of Embodiments 197-231, wherein a content of the active agent in the form is from about 0.0001 wt % to about 50 wt %.

### Embodiment 233

The dosage form according to any of Embodiments 197-232, wherein the form provides a bioavailability of the active agent of at least about 0.25% as measured by an Endoscopic Bioavailability Assay.

### Embodiment 234

The dosage form according to any of Embodiments 197-233, further comprising an osmagent in a content of from about 1 wt % to about 60 wt %.

### Embodiment 235

The dosage form according to Embodiment 234, wherein the osmagent comprises at least one of sucrose, mannitol, fructose and polyethylene glycol.

### Embodiment 236

The dosage form according to any of Embodiments 197-236, further comprising a permeation enhancer in a content of about 0.1 wt % to about 20 wt %.

### Embodiment 237

The dosage form according to Embodiment 236, wherein the permeation enhancer comprises at least one of EDTA, palmitoyl carnitine, dimethyl palmitoyl ammonio propanesulfonate and sodium caprate.

### Embodiment 238

A method of delivering an active agent to a subject, the method comprising:
administering a pharmaceutically acceptable oral dosage form comprising the active agent, at least one drying agent, and a protective coating,
wherein the dosage form has a total drying agent content of at least about 15% by weight,
wherein the protective coating is formulated to release the active agent and at least one drying agent at an intestinal site, and
wherein the form provides a drying effect in an area about the intestinal site.

### Embodiment 239

The method according to Embodiment 238, wherein the at least one drying agent has a drying capacity according to an Agent Drying Capacity Assay of at least about 3 mg fluid/mg drying agent.

### Embodiment 240

The method according to any one of Embodiments 238-239, wherein the drying agent has a drying time according to an Agent Drying Time Assay of at least about 60 seconds.

### Embodiment 241

The method according to any of Embodiments 238-240, wherein form has a drying capacity as measured for the entire form according to a Dosage Form Drying Capacity Assay of at least about 3 g fluid/oral dosage form.

### INCORPORATION BY REFERENCE

All patents and patent application publications mentioned herein, are hereby incorporated by reference in their entirety for all purposes as if each individual patent and/or patent application publication was specifically and individually incorporated by reference. In case of conflict, the instant application, including any definitions herein, will control.

### EQUIVALENTS

While specific embodiments have been discussed, the above specification is illustrative and not restrictive. Many variations will become apparent to those skilled in the art upon review of this specification. The full scope of the embodiments should be determined by reference to the claims, along with their full scope of equivalents, and the specification, along with such variations.

What is claimed is:

1. A pharmaceutically acceptable oral dosage form for delivery of an agent to an intestinal site, comprising:
one or more active agent regions comprising an active agent to be delivered to the intestinal site;
one or more drying agent regions comprising at least one drying agent therein capable of drying an area about the intestinal site, the one or more drying agent regions being separate from the one or more active agent regions; and
a protective coating at least partially covering a surface of the form, wherein the dosage form has a fluid uptake capacity as measured for the entire dosage form when immersed in a fluid media according to a Dosage Form Fluid Uptake Assay of at least about 20 g fluid per dosage form.

2. The dosage form according to claim 1, wherein the fluid uptake capacity as measured for the entire dosage form when immersed in the fluid media according to the Dosage Form Fluid Uptake Assay is at least about 40 g fluid per dosage form.

3. The dosage form according to claim 1 or 2, wherein the fluid uptake capacity as measured for the entire dosage form when immersed in the fluid media according to the Dosage Form Fluid Uptake Assay is at least about 60 g fluid per dosage form.

4. The dosage form according to any preceding claim, wherein a fluid uptake time to reach the fluid uptake capacity, as determined by the Dosage Form Fluid Uptake Time Assay at pH when the dosage form is immersed in fluid media at a pH of 7.4, is no more than 2 hours.

5. The dosage form according to claim 4, wherein the fluid uptake time to reach the fluid uptake capacity, as determined by the Dosage Form Fluid Uptake Time Assay at pH when the dosage form is immersed in fluid media at a pH of 7.4, is no more than 30 minutes.

6. The dosage form according to claim 4 or 5, wherein the fluid uptake time to reach the fluid uptake capacity, as determined by the Dosage Form Fluid Uptake Time Assay at pH when the dosage form is immersed in fluid media at a pH of 7.4, is no more than 5 mins.

7. The dosage form according to any preceding claim, wherein a fluid uptake time to increase the total fluid uptake (MU_{D}) of the dosage form by 50%, as determined by the Dosage Form Fluid Uptake Time Assay for Uptake Phase, is no more than 2 hours.

8. The dosage form according to claim 7, wherein the fluid uptake time to increase the total fluid uptake (MU_{D}) of the dosage form by 50%, as determined by the Dosage Form Fluid Uptake Time Assay for Uptake Phase, is no more than 30 minutes.

9. The dosage form according to claim 8, wherein the fluid uptake time to increase the total fluid uptake (MU_{D}) of the dosage form by 50%, as determined by the Dosage Form Fluid Uptake Time Assay for Uptake Phase, is no more than 5 minutes.

10. The dosage form according to any of the preceding claims, wherein a fluid uptake time to reach the fluid uptake capacity for the dosage form, in a case where interior contents thereof are exposed to the fluid media, as determined by the Dosage Form Fluid Uptake Time Assay at Breakthrough, is no more than 2 hours.

11. The dosage form according to claim 10, wherein a fluid uptake time to reach the fluid uptake capacity for the dosage form, in a case where the interior contents thereof are exposed to the fluid media, as determined by the Dosage Form Fluid Uptake Time Assay at Breakthrough, is no more than 30 minutes.

12. The dosage form according to claim 11, wherein a fluid uptake time to reach the fluid uptake capacity for the dosage form, in a case where the interior contents thereof are exposed to the fluid media, as determined by the Dosage Form Fluid Uptake Time Assay at Breakthrough, is no more than 5 minutes.

13. The dosage form according to any of the preceding claims, wherein the at least one drying agent has a fluid uptake capacity when immersed in a fluid media, according to an Agent Fluid Uptake Assay, of at least about 20 (mg fluid media/mg drying agent), and wherein a Media Uptake Ratio (MUR) used to determine the fluid uptake capacity of the at least one dosage form in the Agent Fluid Uptake Assay is defined using the following formula:
MUR=(F₀−Fᵣ)/P;
where F₀−Fᵣ is the mass of fluid absorbed by the at least one drying agent in the Agent Fluid Uptake Assay, and P is the initial mass of the at least one drying agent prior to immersion in the fluid media in the Agent Fluid Uptake Assay.

14. The dosage form according to claim 13, wherein the at least one drying agent has a fluid uptake capacity when immersed in a fluid media, according to the Agent Fluid Uptake Assay, of at least about 40.

15. The dosage form according to any of claims 13-14, wherein the at least one drying agent has a fluid uptake capacity when immersed in a fluid media, according to the Agent Fluid Uptake Assay, of at least about 60.

16. The dosage form according to any preceding claim, wherein the at least one drying agent has a fluid uptake time to reach its fluid uptake capacity, as determined by an Agent Fluid Uptake Time Assay when the at least one drying agent is immersed in a fluid media, of no more than 30 minutes.

17. The dosage form according to claim 16, wherein the at least one drying agent has a fluid uptake time to reach its fluid uptake capacity, as determined by an Agent Fluid Uptake Time Assay when the at least one drying agent is immersed in a fluid media, of no more than 15 minutes.

18. The dosage form according to claim 16 or 17, wherein the at least one drying agent has a fluid uptake time to reach its fluid uptake capacity, as determined by an Agent Fluid Uptake Time Assay when the at least one drying agent is immersed in a fluid media, of no more than 1 minute.

19. The dosage form according to any preceding claim, wherein the one or more active agent regions and the one or more drying agent regions comprise discrete boundaries therebetween.

20. The form according to any preceding claim, wherein the one or more active agent regions comprise less than 30 wt % of the at least one drying agent, and one or more drying agent regions comprise less than 20 wt % of the at least one active agent.

21. The dosage form according to any preceding claim, wherein the one or more drying agent regions comprise one or more elements having the at least one drying agent therein.

22. The dosage form according to any preceding claim, wherein the one or more drying agent regions comprising one or more of a layer, tablet, particle, granule, bead, bulk polymeric matrix, and combinations thereof.

23. The dosage form according to any preceding claim, wherein the one or more active agent regions comprise one or more elements having the at least one active agent therein.

24. The dosage form according to any preceding claim, wherein the one or more active agent regions comprise one or more of a layer, tablet, particle, granule, bead, lipophilic vehicle, emulsion, suspension, solution, semi-solid, liquid and combinations thereof.

25. The dosage form according to any of claims 21-24, wherein the one or more elements each comprise a unitary structure.

26. The dosage form according to any of claims 21-25, wherein the dosage form is in the form of a capsule, optionally comprising the one or more elements therein.

27. The dosage form according claim 26, wherein one or more of the elements are in the form of a tablet inside the capsule that is compressed by applying a pressure of at least about 5000 psi, and no more than about 18000 psi.

28. The dosage form according to claim 27, wherein the one or more tablets are compressed by applying a pressure of no more than about 12000 psi.

29. The dosage form according to claim 27 or claim 28, wherein a density of the tablets (mg tablet/volume tablet) is at least about 0.7 mg/mm³, and no more than about 1.05 mg/mm³.

30. The dosage form according to claim 29, wherein a density of the tablets (mg drying agent/volume tablet) is no more than about 0.90 mg/mm³.

31. The dosage form according to of claims 1-20, wherein the dosage form is in the form of a compressed tablet having separate one or more active agent regions and one or more drying agent regions.

32. The dosage form according to claim 31, wherein the dosage form is compressed by applying a pressured of at least about 5000 psi, and no more than about 18000 psi

33. The dosage form according to claim 32, wherein the dosage form is compressed at a compression force of no more than about 10000 psi.

34. The dosage form according to any of claims 32-33, wherein a density of the dosage form, in mg of dosage form per volume of dosage form, is at least about 0.7 mg/mm³, and no more than about 1.05 mg/mm³.

35. The dosage form according to claim 34, wherein a density of the dosage form, in mg of dosage form per volume of dosage form, is no more than about 0.90 mg/mm³.

36. The dosage form according to claims 1-20 and 31-35, wherein the dosage form comprises a compressed tablet having separate layers corresponding to the one or more active agent regions and the one or more drying agent regions.

37. The dosage form according to claim 36, wherein the dosage form comprises a barrier layer between layers.

38. The dosage form according to claim 37, wherein the barrier layer is provided to the dosage form in a weight of in a range of from 40 mg to 400 mg per dosage form.

39. The dosage form according to claim 38, wherein the barrier layer is provided to the dosage form in a weight of in a range of from 50 mg to 150 mg per dosage form.

40. The dosage form according to any of claims 36-37, wherein the separate layers comprise one or more of upper and lower layers and concentric layers.

41. The dosage form according to any of claims 1-30, wherein the dosage form comprises capsule form with one or more active agent regions that comprise a lipophilic vehicle having the active agent therein.

42. The dosage form according to claim 41, wherein the one or more active agent regions comprise a lipophilic liquid having the at least one active agent dissolved or suspended therein.

43. The dosage form according to any of claims 41-42, wherein the one or more active agent regions comprise a lipophilic vehicle comprising at least one of a wax, oil, gel, semi-solid and paste.

44. The dosage form according to any of claims 41-43, wherein the one or more active agent regions comprise a lipophilic vehicle that is a solid at room temperature, and is at least partially in liquid form at physiological temperatures.

45. The dosage form according to any of claims 41-44, wherein the lipophilic vehicle having the at least one active agent therein is encapsulated in an interior capsule body.

46. The dosage form according to any of claims 41-45, wherein the lipophilic vehicle in the one or more active agent regions comprises less than about 2 wt % of water.

47. The dosage form according to any preceding claim, comprising at least one active agent region located at a peripheral end of the dosage form.

48. The dosage form according to any preceding claim, comprising at least one drying agent region located at a peripheral end of the dosage form.

49. The dosage form according to any preceding claim, comprising at least one active agent region at a first end of the dosage form, and at least one drying agent region at an opposing second end of the dosage form.

50. The dosage form according to any preceding claim, comprising active agent regions located at opposing peripheral ends of the dosage form, and comprising at least one drying agent region between the active agent regions.

51. The dosage form according to any preceding claim, comprising drying agent regions located at opposing peripheral ends of the dosage form, and comprising at least one active agent region located between the drying agent regions.

52. The dosage form according to any preceding claim, wherein the dosage form comprises a plurality of alternating active agent regions and drying agent regions, alternating along a longitudinal axis of the dosage form.

53. The dosage form according to any preceding claim, wherein the one or more active agent regions comprise a permeation enhancer.

54. The dosage form according to any preceding claim, comprising a permeation enhancer that is one or more of a fatty acid, medium chain glyceride, surfactant, non-steroidal detergent, acyl carnitine, lauroyl carnitine, palmitoyl carnitine, alkanoyl carnitine, N-acetylated amino acid, esters, salts, bile salts, sodium salts, nitrogen-containing rings, and derivatives and combinations thereof.

55. The dosage form according to claim 54, wherein the permeation enhancer is selected from the group consisting of sodium caprate, lauroyl carnitine, palmitoyl carnitine, and 3-(N,N-dimethylpalmitylammonio)propanesulfate (PPS).

56. The dosage form according to any preceding claim, comprising a permeation enhancer having a content of at least 5 mg per dosage form and no more than 800 mg per dosage form.

57. The dosage form according to claim 56, wherein the permeation enhancer is provided in a content of at least 5 mg per dosage form and no more than 50 mg per dosage form.

58. The dosage form according to claim 56, wherein the permeation enhancer is provided in a content of at least 50 mg per dosage form and no more than 200 mg per dosage form.

59. The dosage form according to any preceding claim, wherein a ratio by volume of the one or more drying agent regions to the one or more active agent regions is in a range of from 10:1 to 0.1:1.

60. The dosage form according to any preceding claim, wherein the form has a drying capacity as measured for the entire form according to a Dosage Form Drying Capacity Assay of at least about 20 g fluid/oral dosage form.

61. The dosage form according to claim 60, wherein the form has a drying capacity as measured for the entire form according to a Dosage Form Drying Capacity Assay of at least about 40 g fluid/oral dosage form.

62. The dosage form according to any preceding claim, wherein the at least one drying agent has a drying capacity according to an Agent Drying Capacity Assay of at least about 20 mg fluid/mg drying agent.

63. The dosage form according to claim 62, wherein the at least one drying agent has a drying capacity according to an Agent Drying Capacity Assay of at least about 40 mg fluid/mg drying agent.

64. The dosage form according to any preceding claim, wherein the at least one drying agent has a drying time as measured by an Agent Drying Time Assay of less than 1800 seconds.

65. The dosage form according to claim 64, wherein the at least one drying agent has a drying time as measured by an Agent Drying Time Assay of less than 900 seconds.

66. The dosage form according to any preceding claim, wherein the at least one drying agent comprises at least one selected from the group consisting of disintegrants, super-disintegrants, dessicants, super-absorbent polymers, swellable polymers, and super porous hydrogels and the like.

67. The dosage form according to claim 66, wherein the at least one drying agent comprises at least one selected from the group consisting of modified cellulose/crosslinked cellulose and derivatives thereof, croscarmellose sodium, carboxymethyl cellulose calcium, carboxymethyl cellulose sodium, hydroxypropyl cellulose, methyl cellulose, povidone, crosslinked polyvinylpyrrolidone, starch and/or modified starch, crosslinked starch, crosslinked alginic acid, sodium polyacrylate, cross-linked sodium polyacrylate, sodium starch glycolate, soy polysaccharide, gellan gum, xanthan gum, silicon dioxide, magnesium aluminum silicate, calcium silicate, and ion exchange resins.

68. The dosage form according to claim 67, wherein the at least one drying agent is selected from the group consisting of sodium carboxymethyl starches, cross carmellose, cross-linked sodium polyacrylate, crospovidone, and sodium starch glycolate.

69. The dosage form according to any of claims 1-68, wherein the at least one drying agent is comprises a polymer hydrogel having a hydrophilic polymer cross-linked with a polycarboxylic acid.

70. The dosage form according to any preceding claim, wherein the dosage form has a total drying agent content of at least about 10% by weight.

71. The dosage form according to any preceding claim, wherein the dosage form has a total drying agent content of at least about 15% by weight.

72. The dosage form according to any preceding claim, wherein the dosage form has a total drying agent content of at least about 30% by weight.

73. The dosage form according to any preceding claim, wherein the dosage form has a total drying agent content of at least about 50% by weight.

74. The dosage form according to any preceding claim, wherein the dosage form has a total drying agent content of at least about 75% by weight.

75. The dosage form according to any preceding claim, wherein the active agent comprises at least one selected from the group consisting of peptides, peptides structurally engineered to resist enzymatic degradation, antibodies, hormones, enzymes, growth factors, organic molecules, inorganic molecules, ligands, pharmaceuticals, nutraceuticals, biologics, metals, metal oxides, proteins, protein conjugates, monoclonal antibodies, polyclonal antibodies, antibody fragments, polysaccharides, carbohydrates, nanoparticles, vaccines, nucleic acids, cells and cell therapies, DNA, RNA, siRNA, blood factors, gene therapies, thrombolytic agents, growth factors, interferons, interleukin-based molecules, fusion proteins, recombinant proteins, therapeutic enzymes, drug conjugates, and metabolites.

76. The dosage form according to claim 75, wherein the active agent comprises at least one selected from the group consisting of octreotide, calcitonin, parathyroid hormone (PTH), teriparatide, insulin, exenatide, liraglutide, lixisenatide, albiglutide and dulaglutide.

77. The dosage form according to claim 75 or 76, wherein the active agent comprises a molecular weight of at least about 450 Da and less than about 10000 Da.

78. The dosage form according to any of claims 75-77, wherein the active agent comprise a molecular weight in the range of from about 1000 Da to about 5000 Da.

79. The dosage form according to any preceding claim, wherein the protective coating is capable of becoming at least partially permeable upon exposure to fluid at the intestinal site, and wherein at least 35% of the surface area of the protective coating becomes permeable upon exposure to the fluid at the intestinal site.

80. The dosage form according to any preceding claim, wherein a portion of the protective coating that becomes at least partially permeable upon exposure to fluid covers at least 35% of the drying agent region.

81. The dosage form according to any of claims 79-80, wherein substantially the entire surface of the protective coating covering the drying agent region becomes at least partially permeably upon exposure to fluid at the intestinal site.

82. The dosage form according to any preceding claim, wherein the protective coating comprises an enteric coating that becomes at least partially permeable and/or dissolves at a pH in the range of from 5.5 to 7.5.

83. The dosage form according to any preceding claim, wherein the enteric coating becomes at least partially permeable and/or dissolves at a pH of at least 5.5.

84. The dosage form according to claim 83, wherein the enteric coating becomes at least partially permeable and/or dissolves at a pH of at least 6.5.

85. The dosage form according to claim 84, wherein the enteric coating becomes at least partially permeable and/or dissolves at a pH of at least 7.4.

86. The dosage form according to any preceding claim, wherein the dosage form provides a release rate of the active agent of at least 90% within 30 mins, as determined by a USP Dissolution Assay 711 with Apparatus 1 and a dissolution medium of 150 mM phosphate buffered saline at a pH of 7.5.

87. The dosage form according to any preceding claim, wherein the dosage form provides a release rate of the active agent of at least 90% within 10 mins, as determined by a USP Dissolution Assay 711 with Apparatus 1 and a dissolution medium of 150 mM phosphate buffered saline at a pH of 7.5.

88. The dosage form according to any preceding claim, wherein the dosage form provides a release rate of the active agent of at least 90% within 1 minute, as determined by a USP Dissolution Assay 711 with Apparatus 1 and a dissolution medium of 150 mM phosphate buffered saline at a pH of 7.5

89. A method of delivering an active agent to a subject, the method comprising:
administering the pharmaceutically acceptable dosage form of any of the preceding claims to the subject,
wherein the form provides a drying effect about the intestinal site for delivery of the active agent.

90. A pharmaceutically acceptable oral dosage form for delivery of an agent to an intestinal site comprising:
an active agent to be delivered to the intestinal site;
at least one drying agent therein capable of drying an area about the intestinal site;
at least one permeation enhancer to enhance absorption of the active agent at the intestinal site; and
a protective coating at least partially covering a surface of the form,
wherein the dosage form has a fluid uptake capacity as measured for the entire dosage form when immersed in a fluid media according to a Dosage Form Fluid Uptake Assay of at least about 20 g fluid per dosage form.

91. The dosage form according to claim 90, wherein a total permeation enhancer content in the dosage form is in a range of from at least 5 mg per dosage form to no more than 800 mg per dosage form.

92. The dosage form according to claim 91, wherein the total permeation enhancer content in the dosage form is in a range of from at least 5 mg to no more than 50 mg per dosage form

93. The dosage form according to claim 91, wherein the total permeation enhancer content in the dosage form is at least 50 mg and no more than 200 mg per dosage form

94. The dosage form according to any of claims 90-93, wherein the permeation enhancer that is one or more of a fatty acid, medium chain glyceride, surfactant, non-steroidal detergent, acyl carnitine, lauroyl carnitine, palmitoyl carnitine alkanoyl carnitine, N-acetylated amino acid, esters, salts, bile salts, sodium salts, nitrogen-containing rings, and derivatives and combinations thereof.

95. The dosage form according to claim 94, wherein the permeation enhancer is selected from the group consisting of sodium caprate, lauroyl carnitine, palmitoyl carnitine and 3-(N,N-dimethylpalmitylammonio)propanesulfate (PPS).

96. The dosage form according to any of claims 90-95, wherein the permeation enhancer is a hydrophilic permeation enhancer.

97. The dosage form according to any of claims 90-95, wherein the permeation enhancer is a hydrophobic permeation enhancer.

98. The dosage form according to any of claims 90-97, wherein the permeation enhancer has a log P of at least 2.

99. The dosage form according to any of claims 90-97, wherein the permeation enhancer has a log P of less than 4.

100. The dosage form according to any of claims 90-99, wherein the permeation enhancer comprises sodium caprate, and is provided in an amount of at least 10 mg per dosage form, and no more than 50 mg per dosage form.

101. The dosage form according to claim 100, wherein the sodium caprate is provided in an amount of less than 35 mg per dosage form.

102. The dosage form according to any of claims 90-99, wherein the permeation enhancer comprises PPS, and is provided in an amount of at least 10 mg per dosage form, and no more than 50 mg per dosage form.

103. The dosage form according to claim 102, wherein the PPS is provided in an amount of less than 35 mg per dosage form.

104. The dosage form according to any of claims 90-103, wherein the at least one permeation enhancer is one or more active agent regions of the form comprising the active agent therein, and the at least one drying agent is in one or more drying agent regions of the form, the one or more active agent regions being separate from the one or more drying agent regions.

105. The dosage form according to any of claims 90-104, comprising one or more active agent regions comprising less than 30 wt % of the at least one drying agent therein, and comprising one or more drying agent regions comprising less than 20 wt % of the at least one active agent therein.

106. The dosage form according to any of claims 90-105, further comprising an extended release agent to extend release of one or more of the active agent and permeation enhancer from the dosage form.

107. The dosage form according to claim 106, wherein the extended release agent comprises at least one selected from the group consisting of pectin, hydroxypropyl methyl cellulose, acrylic acid polymer and co-polymers, acacia, alginic acid, polyvinyl alcohol, sodium alginate, tragacanth, methylcellulose, poloxamers, carboxymethyl cellulose, and ethylcellulose.

108. The dosage form according to any of claims 90-107, comprising one or more drying agent regions comprising one or more selected from the group consisting of layers, tablets, granules, powders, beads, bulk polymeric matrices, and combinations thereof.

109. The dosage form according to any of claims 90-108, comprising one or more active agent regions comprising one or more selected from the group consisting of layers, tablets, granules, powders, beads, lipophilic vehicles, emulsions, suspensions, solutions, semi-solids, liquids, and combinations thereof.

110. The dosage form according to any of claims 90-109, wherein the dosage form comprises a capsule form having one or more drying agent regions comprising one or more tablets having the at least one drying agent therein.

111. The dosage form according to any of claims 90-110, wherein the dosage form comprises a capsule form comprising one or more active agent regions comprising one or more tablets having the at least one active agent and permeation enhancer therein.

112. The dosage form according to any of claims 90-111, wherein the dosage form comprises a capsule form comprising one or more active agent regions comprising a lipophilic vehicle having the at least one active agent and permeation enhancer therein.

113. The dosage form according to any of claims 90-112, wherein the dosage form comprises a capsule form having at least one of the active agent, drying agent, and permeation enhancer in the form of at least one of a powder, granule and bead.

114. The dosage form according to any of claims 90-113, wherein the form comprises a tablet having the at least one drying agent, at least one active agent, and at least one permeation enhancer therein.

115. The dosage form according to any of claims 90-114, wherein the permeation enhancer is located in region at a peripheral end of the dosage form.

116. The dosage form according to any of claims 90-115, wherein the permeation enhancer is located in an interior region of the dosage form.

117. The dosage form according to any of claims 90-116, wherein at least a portion of the at least one permeation enhancer is located at a first peripheral end of the dosage form, and at least a portion of the at least one drying agent is located at a second peripheral end of the dosage form, the first and second ends opposing one another.

118. The dosage form according to any of claims 90-117, wherein the permeation enhancer is located in a region in an interior of the dosage form, the interior region being in between exterior regions containing the at least one drying agent.

119. The form according to any of claims 90-118, wherein the permeation enhancer and at least one drying agent are provided in regions that alternate along a longitudinal axis of the dosage form.

120. The dosage form according to any of claims 90-119, wherein the fluid uptake capacity as measured for the entire dosage form when immersed in the fluid media according to the Dosage Form Fluid Uptake Assay is at least about 40 g fluid per dosage form.

121. The dosage form according to claim 120, wherein the fluid uptake capacity as measured for the entire dosage form when immersed in the fluid media according to the Dosage Form Fluid Uptake Assay is at least about 60 g fluid per dosage form.

122. A method of delivering an active agent to a subject, the method comprising:
administering the pharmaceutically acceptable dosage form of any of claims 90-121 to the subject,
wherein the form provides a drying effect about the intestinal site and enhances absorption of the active agent at the intestinal site.

123. A pharmaceutically acceptable oral dosage form for delivery of an agent to an intestinal site comprising:
at least one active agent to be delivered to the intestinal site,
at least one drying agent provided in a percent by weight of at least 10 wt %; and
a protective coating covering a surface of the form,
wherein the dosage form is compressed at a pressure of at least 5000 psi.
wherein the dosage form has a fluid uptake capacity as measured for the entire dosage form when immersed in a fluid media according to a Dosage Form Fluid Uptake Assay of at least about 20 g fluid per dosage form.

124. The dosage form according to claim 123, wherein the dosage form is compressed at a pressure of no more than about 18000 psi.

125. The dosage form according to claim 124, wherein the dosage form is compressed at a compressive force of no more than about 10000 psi.

126. The dosage form according to any of claims 123-125, wherein the dosage form has a density in mg of dosage form per volume of dosage form that is in the range of from about at least about 0.7 mg/mm³ to no more than about 1.05 mg/mm³.

127. The dosage form according to claim 126, wherein the dosage form has a density that is no more than about 0.90 mg/mm³.

128. The dosage form according to any of claims 123-127, wherein the fluid uptake capacity as measured for the entire dosage form when immersed in the fluid media according to the Dosage Form Fluid Uptake Assay is at least about 40 g fluid per dosage form.

129. The dosage form according to claim 128, wherein the fluid uptake capacity as measured for the entire dosage form when immersed in the fluid media according to the Dosage Form Fluid Uptake Assay is at least about 60 g fluid per dosage form.

130. The dosage form according to any of claims 123-129, wherein the oral dosage form is in the form of at least one of a tablet and caplet.

131. The dosage form according to any of claims 123-130, comprising a first compressed region having the at least one active agent, and a second compressed region having the at least one drying agent.

132. The dosage form according to claim 131, wherein the first and second compressed regions are first and second compressed layers.

133. The dosage form according to any of claims 131-132, wherein the first and second compressed regions are separated by a barrier layer that inhibits contact between the first and second compressed regions.

134. The dosage form according to any of claims 131-133, wherein the first and second compressed regions are separated by a barrier layer that at least partially inhibits penetration of one or more of the first and second compressed region by the other compressed region during dissolution of the dosage form in vivo.

135. The dosage form according to any of claims 131-134, wherein the second compressed region comprises a drying agent that is at least one selected from the group consisting of modified cellulose/crosslinked cellulose and derivatives thereof, croscarmellose sodium, carboxymethyl cellulose calcium, carboxymethyl cellulose sodium, hydroxypropyl cellulose, methyl cellulose, povidone, crosslinked polyvinylpyrrolidone, starch and/or modified starch, crosslinked starch, crosslinked alginic acid, sodium polyacrylate, crosslinked sodium polyacrylate, sodium starch glycolate, soy polysaccharide, gellan gum, xanthan gum, silicon dioxide, magnesium aluminum silicate, calcium silicate, and ion exchange resins.

136. The dosage form according to claim 135, wherein the at least one drying agent is selected from the group consisting of sodium carboxymethyl starches, cross carmellose, crosslinked sodium polyacrylate, crospovidone, and sodium starch glycolate.

137. The dosage form according to any of claims 131-135, wherein the at least one drying agent is comprises a polymer hydrogel having a hydrophilic polymer cross-linked with a polycarboxylic acid.

138. The dosage form according to any of claims 131-135, wherein the second compressed region comprises the at least one drying agent in a content of from 10 wt % to 99 wt % of the second compressed region.

139. The dosage form according to any of claims 131-138, wherein the second compressed region comprises the at least one drying agent in a content of from 50 wt % to 95 wt % of the second compressed region.

140. The dosage form according to any of claims 131-139, wherein the second compressed region comprises less than 20% by weight of the at least one active agent, and the first compressed region comprises less than 30% by weight of the at least one drying agent.

141. The dosage form according to any of claims 131-140, wherein at least one of the first and second compressed regions comprise a protective coating permeability promoter that promotes at least partial dissolution of the protective coating in vivo to achieve release of contents of one or more of the first and second compressed regions.

142. The dosage form according to claim 141, wherein the protective coating permeability promoter comprises a compound that increases the pH about the protective coating.

143. The dosage form according to any of claims 141-142, wherein the protective coating permeability promoter comprises at least one base in powder form.

144. The dosage form according to any of claims 123-143, wherein the protective coating is an enteric coating that becomes at least partially permeable and/or at least partially dissolved at a pH in a range of from 5.5 to 7.5.

145. The dosage form according to any of claims 123-144, wherein the protective coating is an enteric coating that becomes at least partially permeable and/or at least partially dissolved at a pH of at least 5.5.

146. The dosage form according to any of claims 123-145, wherein the protective coating is an enteric coating that becomes at least partially permeable and/or at least partially dissolved at a pH of at least 6.5.

147. The dosage form according to any of claims 123-146, wherein the protective coating is an enteric coating that becomes at least partially permeable and/or at least partially dissolved at a pH of at least 7.4.

148. The dosage form according to any of claims 123-147, wherein the dosage form comprises at least one permeation enhancer that enhances absorption of the active agent at the intestinal site.

149. The dosage form according to claim 148, wherein the permeation wherein the permeation enhancer that is one or more of a fatty acid, medium chain glyceride, surfactant, non-steroidal detergent, acyl carnitine, lauroyl carnitine, palmitoyl carnitine, alkanoyl carnitine, N-acetylated amino acid, esters, salts, bile salts, sodium salts, nitrogen-containing rings, and derivatives and combinations thereof.

150. The dosage form according to claim 148 or 149, wherein the permeation enhancer is selected from the group consisting of sodium caprate, lauryl carnitine, palmitoyl carnitine, and 3-(N,N-dimethylpalmitylammonio)propanesulfate (PPS).

151. The dosage form according to any of claims 148-150, wherein the permeation enhancer is provided in an amount of 5 wt % to 95 wt % as a percentage of the weight of the first compressed region.

152. The dosage form according to any of claims 148-150, wherein the second compressed region comprises a binder material in a percent content of from 1 wt % to 10 wt % of the second compressed region.

153. The dosage form according to any of claims 131-152, wherein the second compressed region comprises a binder material selected from the group consisting of polyvinylpyrrolidone, HPMC, and pectin

154. The dosage form according to any of claims 123-130, wherein the compressed dosage form comprises a substantially uniform mixture of the at least one drying agent and the at least one active agent.

155. The dosage form according to any of claims 123-153, wherein the dosage form comprises a first layer comprising the at least one active agent, and a second layer comprising the at least one drying agent, and wherein a content of active agent in the second layer is less than 20 wt % by weight of the second layer, and a content of drying agent in the first layer is less than 30 wt % by weight of the first layer.

156. The dosage form according to any of claims 121-155, wherein the first and second layers comprise one or more of stacked layers and concentric layers.

157. A pharmaceutically acceptable oral dosage form for delivery of an agent to an intestinal site comprising:
at least one active agent to be delivered to the intestinal site;
at least one drying agent capable of drying an area about the intestinal site; and
a protective coating covering a surface of the form,
wherein the at least one active agent and at least one drying agent are contained in a capsule body having the protective coating on the surface thereof, and
wherein the dosage form has a fluid uptake capacity as measured for the entire dosage form when immersed in a fluid media according to a Dosage Form Fluid Uptake Assay of at least about 20 g fluid per dosage form.

158. The dosage form according to claim 157, wherein one or more of the at least one active agent and the at least one drying agent are contained in a compressed element that is compressed by applying a pressure of at least 5000 psi and no more than 18000 psi.

159. The dosage form according to claim 158, wherein the compressed element is compressed by applying a pressure of no more than 12000 psi.

160. The dosage form according to claim 158 or 159, wherein a density of the compressed element in mg of compressed element per volume of compressed element is in the range of from about 0.7 mg/mm³ to about 1.05 mg/mm³.

161. The dosage form according to claim 160, wherein the density of the compressed element is no more than about 0.90 mg/mm³.

162. The dosage form according to any one of claims 151 to 156, wherein the dosage form comprises one or more active agent regions having the at least one active agent, and one or more drying agent regions having the least one drying agent.

163. The dosage form according to claim 157, wherein the at least one drying agent is provided in a content of at least 20% by weight of the drying agent region.

164. The dosage form according to claim 163, wherein the at least one drying agent is provided in a content of at least 50% by weight of the drying agent region.

166. The dosage form according to claim 165, wherein the at least one drying agent is provided in a content of at least 90% by weight of the drying agent region.

166. The dosage form according to any of claims 157-166, wherein the one or more drying agent regions comprise one or more compressed elements containing the drying agent therein.

167. The dosage form according to any of claims 157-166, wherein the one or more active agent regions comprise one or more compressed elements containing the active agent therein.

168. The dosage form according to any of claims 157-167, wherein the one or more active agent regions comprise at least one of a lipophilic vehicle, emulsion, solution, semi-solid, powder, grains and beads.

169. The dosage form according to any of claims 157-168, wherein the one or more active agent regions comprise a lipophilic vehicle having the active agent therein.

170. The form according to any of claims 157-169, wherein the one or more drying agent regions are separate from the one or more active agent regions, and wherein the one or more drying agent regions comprise less than 20 wt % of active agent and the one or more active agent regions comprise less than 30 wt % of drying agent.

171. The dosage form according to any of claims 157-170, wherein the form comprises a capsule body having from 2 to 10 compressed elements therein.

172. The dosage form according to claim 171, wherein the form comprises a capsule body having from 3 to 6 compressed elements therein.

173. The dosage form according to any of claims 157-172, wherein the form comprises at least one compressed element having the at least one active agent at an interior portion of the form, and comprises at least one compressed element having the at least one drying agent at an exterior portion of the form.

173. The dosage form according to any of claims 157-172, wherein the form comprises at least one compressed element having the at least one drying agent at an interior portion of the form, and at least one compressed element having the at least one active agent at an exterior portion of the form.

174. The dosage form according to any of claims 157-173, wherein compressed elements having the at least one drying agent, and compressed elements having at least one active agent are provided in an alternating arrangement along an axis of the form.

175. The dosage form according to any of claims 157-174, wherein the at least one active agent region comprises a permeation enhancer capable of increasing absorption of the active agent at the intestinal site.

176. The dosage form according to any of claims 157-175, wherein the dosage form comprises at least one compressed element having the at least one active agent and at least one permeation enhancer.

177. The dosage form according to any of claims 157-176, comprising one or more compressed elements having the at least one active agent and at least one permeation enhancer therein, wherein the permeation enhancer comprises at least 80 wt % of at least one compressed element.

178. The dosage form according to any of claims 157-177, wherein the fluid uptake capacity as measured for the entire dosage form when immersed in the fluid media according to the Dosage Form Fluid Uptake Assay is at least about 40 g fluid per dosage form.

179. The dosage form according to claim 178, wherein the fluid uptake capacity as measured for the entire dosage form when immersed in the fluid media according to the Dosage Form Fluid Uptake Assay is at least about 60 g fluid per dosage form.

180. The dosage form according to any of claims 157-179, wherein the dosage form comprises a plurality of compressed elements, each having a substantially uniform mixture of the at least one active agent and the at least one drying agent.

181. The dosage form according to any of claims 157-180, wherein the dosage form comprises a plurality of compressed elements, and at least one of granules, beads and powder.

182. The dosage form according to claim 181, wherein the plurality of compressed elements comprise the at least one drying agent, and the at least one of granules, beads and powder comprises the at least one active agent.

182. The dosage form according to claim 181 or 182, wherein the plurality of compressed elements are at opposing ends of the dosage form, with the at least one of granules, beads and powders in an interior region of the dosage form.

183. The dosage form according to any of claims 157-182, wherein the dosage form comprises a plurality of compressed elements and a lipophilic vehicle.

183. The dosage form according to claim 183, wherein the plurality of compressed elements comprise the at least one drying agent, and lipophilic vehicle comprises the at least one active agent.

184. The dosage form according to claim 183, wherein the plurality of compressed elements are at a first end of the dosage form, and the lipophilic vehicle is at a second opposing end of the dosage form.

185. The dosage form according to any of claims 157-184, wherein the dosage form comprises a plurality of first compressed elements comprising the at least one drying agent, and at least one second compressed element comprising the at least one active agent.

186. The dosage form according to claim 185, wherein the plurality of first compressed elements are at opposing ends of the dosage form, and the at least one second compressed element is at an interior region of the dosage form.

187. The dosage form according to claim 185 or 186, wherein the at least one second compressed element comprises a permeation enhancer and at least one release extending agent to extend release of one or more of the at least one active agent and permeation enhancer from the dosage form.

188. The dosage form according to claims 186-187, wherein the at least one second compressed element has a greater thickness than any one of the plurality of first compressed elements.

189. The dosage form according to any of claims 157-188, wherein the dosage form comprises a plurality of compressed elements, at least one of the compressed elements having at least two layers.

190. The dosage form according to claim 189, wherein the plurality of compressed elements comprise at least one compressed element having a first layer comprising the at least one drying agent, and a second layer comprising the at least one active agent.

191. The dosage form according to claim 189 or 190, wherein one or more of the plurality of compressed elements comprises a second protective coating on a surface of the compressed element.

192. The dosage form according to any of claims 157-159, wherein dosage form comprises first particles comprising the at least one active agent, and second particles comprising the at least one drying agent.

193. The dosage form according to claim 192, wherein the particles comprise at least one of powders, beads, granules or combinations thereof.

194. The dosage form according to claim 192 or 193, wherein the dosage form comprises a substantially homogenous mixture of the first and second particles.

195. The dosage form according to any of claims 193-194, comprising from 5 wt % to 70 wt % of the first particles, and from 20 wt % to 95 wt % of the second particles.

196. The dosage form according any of claims 157-195, wherein the dosage form comprises particles comprising the at least one drying agent having an average particle size of in the range of from about 100 microns to about 400 microns

197. A pharmaceutically acceptable oral dosage form for delivery of an agent to an intestinal site comprising:
an active agent to be delivered to the intestinal site;
at least one drying agent capable of drying an area about the intestinal site; and
a protective coating covering a surface of the form,
wherein the dosage form has a total drying agent content of at least about 15% by weight.

198. The dosage form according to claim 197, wherein the dosage form has a total drying agent content of at least about 30% by weight.

199. The dosage form according to claim 198, wherein the dosage form has a total drying agent content of at least about 50% by weight.

200. The dosage form according to claim 199, wherein the dosage form has a total drying agent content of at least about 75% by weight.

201. The dosage form according to any of claims 198-200, wherein the drying agent has a drying capacity according to an Agent Drying Capacity Assay of at least about 3 mg fluid/mg drying agent.

202. The dosage form according to claim 201, wherein the drying agent has a drying capacity according to an Agent Drying Capacity Assay of at least about 5 mg fluid/mg drying agent.

203. The dosage form according to claim 202, wherein the drying agent has a drying capacity according to an Agent Drying Capacity Assay of at least about 7 mg fluid/mg drying agent.

204. The dosage form according to any of claims 198-203, wherein the drying agent has a drying time according to an Agent Drying Time Assay of no more than about 15 minutes.

205. The dosage form according to claim 205, wherein the drying agent has a drying time according to an Agent Drying Time Assay of no more than about 5 minutes.

206. The dosage form according to claim 205, wherein the drying agent has a drying time according to an Agent Drying Time Assay of no more than about 60 seconds.

207. The dosage form according to any of claims 197-206, wherein the form comprises a drying composition having the at least one drying agent, and wherein a drying capacity of the drying composition according to an Agent Drying Capacity Assay is at least about 3 mg fluid/mg drying composition.

208. The dosage form according to claim 207, wherein the form has a drying capacity as measured for the entire form according to a Dosage Form Drying Capacity Assay of at least about 3 g fluid/oral dosage form.

209. The dosage form according to any of claims 197-208, wherein the drying agent has a solubility in water such that a viscosity of a liquid part of a solution of water containing 5 mg of the drying agent/mL water at standard temperature and pressure is less than 5 cP.

210. The dosage form according to any of claims 196-209, wherein the at least one drying agent comprises at least one of cross carmellose, sodium polyacrylate, crospovidone, and sodium starch glycolate.

211. The dosage form according to any of claims 196-210, further comprising a gelling agent capable of forming a gel upon exposure to an intestinal environment.

212. The dosage form according to claim 211, wherein the gelling agent comprises at least one of pectin, hydroxypropylmethylcellulose and an acrylic acid polymer/copolymer.

213. The dosage form according to claim 212, wherein a content of the gelling agent in the form is from about 1 wt % to about 50 wt %.

214. The dosage form according to any of claims 197-213, wherein the drying agent is provided in a first region of the form, and the active agent is provided in second region of the form.

215. The dosage form according to claim 214, wherein the first and second regions comprise first and second layers of a bi-layer tablet.

216. The dosage form according to claim 215, wherein the first and second regions comprise first and second layers of a tri-layer tablet, the tri-layer tablet further comprising a third layer having a third composition that is the same and/or different from one or more of the first and second layers.

217. The dosage form according to claim 214, wherein the first and second regions are regions of a compression coated tablet having a core and a compression coating at least partially surrounding the core.

218. The dosage form according to claim 217, wherein the core comprises the first region having the drying agent, and the compression coating comprises the second region having the active agent.

219. The dosage form according to claim 218, wherein the core comprises the second region having the active agent, and the compression coating comprises the first region having the drying agent.

220. The dosage form according to claim 214, wherein a gelling agent is contained in at least one of the first region containing the at least one drying agent and the second region containing the active agent.

221. The dosage form according to any of claims 197-220, wherein the form comprises a mono-layer tablet.

222. The dosage form according to any of claims 197-221, wherein at least one of the drying agent and the active agent is provided in a particulate form.

223. The dosage form according to any of claims 197-222, wherein the form comprises a capsule form.

224. The dosage form according to claim 223, wherein at least one of the active agent and the least one drying agent are provided in a powdered form in the capsule.

225. The dosage form according to claim 224, wherein at least one of the active agent and the at least one drying agent are provided in the form of particles in the capsule

226. The dosage form according to claim 225, wherein the particles comprise at least one of spheres and tablets.

227. The dosage form according to claim 226, wherein the capsule comprises a plurality of tablets having the active agent, and wherein the drying agent at least partially surrounds the tablets.

228. The dosage form according to any of claims 197-227, wherein the protective coating comprises at least one of a pH-dependent enteric coating and a timed-release coating.

229. The dosage form according to claim 228, wherein the protective coating is an enteric coating that is capable of releasing the active agent from the form at a pH of from about 5.5 to about 7.5.

230. The dosage form according to claim 229, wherein the enteric coating comprises at least one of poly(methacrylic acid-co-methyl methacrylate) and methacrylic acid ethyl acrylate copolymer.

231. The dosage form according to any of claims 197-230, wherein the active agent comprises at least one of octreotide, calcitonin, parathyroid hormone, teriparatide, insulin, exenatide, liraglutide, lixisenatide, albiglutide and dulaglutide.

232. The dosage form according to any of claims 197-231, wherein a content of the active agent in the form is from about 0.0001 wt % to about 50 wt %.

233. The dosage form according to any of claims 197-232, wherein the form provides a bioavailability of the active agent of at least about 0.25% as measured by an Endoscopic Bioavailability Assay.

234. The dosage form according to any of claims 197-233, further comprising an osmagent in a content of from about 1 wt % to about 60 wt %.

235. The dosage form according to claim 234, wherein the osmagent comprises at least one of sucrose, mannitol, fructose and polyethylene glycol.

236. The dosage form according to any of claims 197-236, further comprising a permeation enhancer in a content of about 0.1 wt % to about 20 wt %.

237. The dosage form according to claim 236, wherein the permeation enhancer comprises at least one of EDTA, palmitoyl carnitine, dimethyl palmitoyl ammonio propanesulfonate and sodium caprate.

238. A method of delivering an active agent to a subject, the method comprising:
administering a pharmaceutically acceptable oral dosage form comprising the active agent, at least one drying agent, and a protective coating,
wherein the dosage form has a total drying agent content of at least about 15% by weight,
wherein the protective coating is formulated to release the active agent and at least one drying agent at an intestinal site, and
wherein the form provides a drying effect in an area about the intestinal site.

239. The method according to claim 238, wherein the at least one drying agent has a drying capacity according to an Agent Drying Capacity Assay of at least about 3 mg fluid/mg drying agent.

240. The method according to any one of claims 238-239, wherein the drying agent has a drying time according to an Agent Drying Time Assay of at least about 60 seconds.

241. The method according to any of claims 238-240, wherein form has a drying capacity as measured for the entire form according to a Dosage Form Drying Capacity Assay of at least about 3 g fluid/oral dosage form.
